SEQUENCE LISTING (25 sequences)

SEQ ID NO: 1 - protein, 477 aa, Streptomyces sp.
  source 1..477: note = Streptomyces sp. AJS327
MATPSAFSGSVLTPGDDGFEAAQVTWNACYSSRPREVMVCHDAASVAEAVRSVRERGLPFRVRSGGHSMCGLSNLDDGVIIDLGGLGGVELTPDRQTVRIGGGARLADVYNTLWDHRLTVPAGTCPRIGVGGHVLGGGMGVLSRSRGALVDHLTALEMVDAEGRLLRVSEDENPDLFWACRGGGGGNFGIVTAYELRPTPIDDVTIFTVSWTWSQLPDAVRAWQRWLGSAESRINSFLSLFPQQQDMVVAFGVFDGPAADFRPLLAPLTAEVAPEAEVVEEVPFIQAVDTVEALQGEAAAAEQVRAQGSSAIIANPLNDEALATLQEFLTDPPSHRAEVAVYGMGGVIGERERGDTAFVHRTGLMAFEYRTDWDSPEDDRLNLDWVTRLRHAMAEHTTGAAYVNTIDLALENWLWAYYEENLPRLMAVKRRYDPENVFHHPHSIPGSLTAEAARAHGVPEATLKRLHDDGLLDGPLD

SEQ ID NO: 2 - protein, 473 aa, Phytohabitans suffuscus
MSTAPVFGGRIVTRLDPSYEEARQVWNAVPDHRPLEIRYCRDAQDVAAALRDALARGLPFAARSGGHSAAALSVVPDGVVIDVSPLNSISLDQDASQVTFGSGATLGAIYRTLWESGVTVPAGTCPNIGVAGHVLGGGLGILSRSRGPLVDHLIGVTMVDARGELVTADEENHSDLLWACRGGGGGNYGIATSFTLRTQPIADVTLFNITWRFDDLGPAVKAWQSWLADADHRINPFMMLYPREQDMVATVGVMEGTPDAFEPLVKPLLDAVEPSEVVIEPMSFIQAVDTVEALQGEAAVAKSVRALGSSAIIDRPLDDRALETLRRHLIDPPSHRSEVAIYGMGGKIAAKGREETAFVHRTELMAFEYRTDWDEPADDEKNVAWVRSVRQEMAEHTTGGTYTNTVDLGVENWLWGYYEENLPRLMGVKHRYDPDGVFQHAHSVPLSLTEAEASRFGIPEQIRRTLRAAGRLT

SEQ ID NO: 3 - protein, 475 aa, Streptomyces varsoviensis
MATSARFAGTVLAPGDPGFEDASRTWNACFASRPREVMVCHSTAEVADAVREVRERGVSFRVRSGGHSMAGLSSVEDGVVIDLGGLDAISLSADRSLVTVGGGTRLGDIYRELWRSGVTVPAGTCPRIGIGGHVLGGGMGVLSRSRGALVDHLAALEFVDAEGRAYRVDENEHPDLFWACRGGGGGSYGIATSYTLRTSPVDEVTLFTLSWNWERFAPAVRAWQSWLASTDDRVNTFLSLFPRQQDLVAAFGVYDGPAAEFEELLRPLVAAVRPQDQVVEPMPFIQAVDTVELLQGEAAAADQVRAQGSSAIIGGPLDGAAIATLQQFLTDPPSHRSEVAVYGMGGAIARPARTDTAFVHRTGLMAFEYRTDWDRSEDDAKNLAWIKALRHAMAPHTTGAAYVNTIDLALENWLWAYYEENLPRLIEVKRRYDPGDVFHHPHSIPLTVTEADIARYGIPEAVAARLRAAGLTDRQ

SEQ ID NO: 4 - DNA, 1422 bp, Phytohabitans suffuscus
  source 1..1422: mol_type = genomic DNA
atgagcaccgcgcccgttttcggcgggaggatcgtcacccggctggacccctcgtacgaggaggcccgccaggtgtggaacgccgtgcccgaccaccgtccacttgagatcaggtactgccgggacgcgcaggacgtggcggcggcgctgcgcgacgcgctggcgcggggcctgccgttcgccgcccgctcgggtgggcactcggcggccgccctgtcagtggtcccagacggcgtggtgatcgacgtctcgccgttgaactcgatcagcctcgaccaggacgcctcgcaggtgaccttcggcagcggtgcgacgctcggcgccatctaccgcacgctgtgggagtcgggcgtgaccgtaccggcgggcacgtgcccgaacatcggcgtcgcgggccacgtgctcggcggtggcctgggcatcctctcccgctcgcgcggcccgctcgtcgatcacctgatcggggtgacgatggtcgacgcgcgcggcgagcttgtcacggcggacgaggagaaccactcggatctgctgtgggcctgccgtggtggcgggggtggcaactacggcatcgccacctcgttcacccttcgaacccagcccatcgccgatgtcaccctgttcaacatcacctggcggttcgacgacctcggccccgcggtaaaggcatggcagagctggctggccgacgccgaccaccggatcaacccgttcatgatgctctacccgcgcgagcaggacatggtcgccacggtgggtgtcatggagggcacgcccgacgcgttcgagccgttggtcaagccgttgttggacgccgtggagccgtccgaggtggtcatcgagccgatgtcgttcatccaggcggtggacaccgtcgaggcgttgcagggagaggcggctgtcgccaagtccgtccgggcgctgggcagctcggcgatcatcgatcggccgctggacgaccgggcccttgagaccctgcggcgccacctgatcgacccgcccagtcaccgaagtgaggtggcgatctacggcatgggcgggaagatcgccgccaagggtcgcgaggagacggcgttcgtgcaccgtacggagctgatggccttcgagtaccgcaccgactgggacgagccggcggacgacgagaagaacgtggcctgggtgcggagcgtacggcaggagatggccgagcacaccaccggtggcacgtacacgaacaccgtcgatctcggggtcgagaactggctgtggggctactacgaggagaacctgccgcggttgatgggggtcaagcaccgctacgaccccgacggggtcttccaacatgcccacagcgtcccgctgtcgttgaccgaggcggaggcgagccgcttcgggattccagagcagatccgccggaccctacgcgcggccggccgcctgacctga

SEQ ID NO: 5 - DNA, 1428 bp, Streptomyces varsoviensis
  source 1..1428: mol_type = genomic DNA
atggcaacctccgcacgtttcgccggtacggtcctcgcccccggggaccccggattcgaggacgcgagccgcacctggaacgcgtgcttcgcctcccgcccccgcgaggtgatggtgtgtcacagcacggcggaggtggcggacgcggtgcgcgaggtgcgggagcgcggcgtatcgttccgggtccggtcgggaggccactccatggccgggctgagcagcgtcgaggacggggtcgtcatcgacctcggcggcctcgacgccatctccctcagcgcggaccgctcgctggtgaccgtcggcggcggaacccgactcggcgacatctaccgggagttgtggcggtccggcgtgacggtaccggcgggcacctgcccgcgcatcggcatcggcggccatgtgctgggcggcggcatgggcgtgctgtcccgctcccgcggcgccctcgtcgaccacctcgccgccctggagttcgtcgacgccgagggccgggcgtaccgggtggacgagaacgagcaccccgacctgttctgggcctgccggggcgggggtggcgggagctacggcatcgccacctcgtacacgctgcgcacgtccccggtggacgaggtcaccctcttcaccctgagctggaactgggaacggttcgcccccgccgtacgggcgtggcagagctggctggcgtcgaccgacgaccgggtcaacaccttcctctcgctcttcccgcgccagcaggacctggtggcggccttcggcgtgtacgacgggccggccgccgagttcgaggagctgctgcggccgctggtcgccgccgtacggccgcaggaccaggtcgtggagccgatgccgttcatccaggccgtcgacaccgtggaactgctccagggcgaggccgcggcggccgaccaggtacgggcgcagggcagttcggccatcatcggcgggccgctggacggcgcggccatcgccaccctccagcagttcctcaccgacccgccgagccaccgctccgaggtcgccgtctacggcatgggcggggccatcgcgcgcccggcgcgcaccgacacggcgttcgtgcaccggacggggctgatggccttcgagtaccgcaccgactgggaccggtccgaggacgacgcgaagaacctcgcctggatcaaggcgctgcgccacgccatggccccgcacaccaccggcgccgcgtacgtcaacaccatcgacctggccctggagaactggctctgggcctactacgaggagaacctgccgcggctgatcgaggtcaagcgccgctacgaccccggcgacgtcttccaccacccgcacagcatcccgctcacggtcaccgaggcggacatcgcccggtacggcataccggaagcggtggccgcacggctgcgggcggcagggctgacggaccgtcagtag

SEQ ID NO: 6 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 7 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 8 - protein, 305 aa, Cannabis cryptic virus
MSGAADVERVYAAMEEAAGLLGVTCAREKIYPLLTEFQDTLTDGVVVFSMASGRRSTELDFSISVPTSQGDPYATVVDKGLFPATGHPVDDLLADTQKHLPVSMFAIDGEVTGGFKKTYAFFPTDDMPGVAQLSAIPSMPSSVAENAELFARYGLDKVQMTSMDYKKRQVNLYFSELSEQTLAPESVLALVRELGLHVPTELGLEFCKRSFSVYPTLNWDTGKIDRLCFAVISTDPTLVPSTDERDIEQFRHYGTKAPYAYVGENRTLVYGLTLSPTEEYYKLGAYYHITDIQRRLLKAFDALED

SEQ ID NO: 9 - DNA, 1434 bp, Streptomyces sp.
  source 1..1434: mol_type = genomic DNA; note = Streptomyces sp. AJS327
atggccaccccatccgcattctccggcagcgtcctcacccccggtgacgacggcttcgaggcggcccaggtcacctggaacgcctgctactcctcccgcccccgggaggtgatggtctgccacgacgccgcgtccgtcgccgaggcggtgcgcagcgtgcgggagcgcggactgcccttccgggtgcgctccggcggccactcgatgtgcgggctgagcaacctggacgacggcgtcatcatcgacctcggcggcctgggcggcgtcgaactcaccccggaccggcagacggtgcggatcggcggcggcgcccgcctcgccgacgtctacaacaccctctgggaccaccggctgaccgtcccggccggcacctgcccgcggatcggggtcggcggccacgtgctgggcggcggcatgggcgtgctgtcccggtcgcgtggcgcgctggtggaccatctgacggcactggagatggtggacgccgagggccggctcctgcgcgtcagcgaggacgagaaccccgacctgttctgggcctgccggggcggcggtggcggcaacttcggcatcgtgaccgcgtacgagctgcggcccacgccgatcgacgacgtcaccatcttcacggtcagctggacctggtcgcagctccccgacgcggtgcgggcctggcagcggtggctgggctcggcggagagccgtatcaacagcttcctctcactcttcccccaacagcaggacatggtcgtcgcgttcggcgtcttcgacggtccggccgccgacttccggccgctgctggccccgctgaccgccgaggtcgccccggaggccgaggtcgtggaggaggtcccgttcatccaggccgtggacaccgtcgaggcgttgcagggcgaggccgccgccgccgagcaggtgcgggcgcagggcagttcggcgatcatcgcgaacccgctgaacgacgaggcgctggccacgctccaggagttcctcaccgacccgcccagccaccgagcggaggtggccgtctacgggatgggcggcgtcatcggcgaacgggagcgcggcgacacggcgttcgtgcaccgcaccggcctgatggccttcgagtaccgcaccgactgggacagccccgaggacgaccggctcaacctggactgggtgacccggctgcggcacgcgatggccgaacacaccaccggcgccgcctacgtcaacaccatcgacctggccctggagaactggctgtgggcctactacgaggagaacctgccgcgcctgatggcggtcaagcggcggtacgacccggagaacgtcttccaccacccgcacagcatccccggctcgctcaccgccgaggcggcccgggcgcacggcgttcccgaggccaccctcaagcggctgcacgacgacggcctgctcgacggaccgctggactga

SEQ ID NO: 10 - protein, 473 aa, Streptomyces olivochromogenes
MGTPARFTGTVLTPGDAGFEDASRTWNACYASRPREVMVCHSAEAVAEALGSARGRGIPFRVRSGGHSMAGLSSVADGAIIDLGGLDAITLSEDSSLVTVGGGARLGDIYRELWKAGVTVPAGTCPRIGIGGHVLGGGMGVLSRSRGALVDHLVGLELVGADGRLRQVDDDEHADLFWACRGGGGGSFGIATSYTFRTSPISEVTIFTIAWSWAQFAPAVKAWQGWLASAEDRINTFLSLFPRQQDLLVAFGVFDGPAAEFEELLRPLVAAAEPQTLVVEPVPFIQAVDTVEALQGEAAAADQVRAQGSSAIVAEPLNEAAIATLQEFLTDPPSHRAEVAVYGMGGAIGHRARTDTAFVHRTGLMAFEYRTDWDLPEDDGKNLAWVRALRHSMAEHTTGAAYVNTIDLALENWLWAYYEENLPRLMQVKRQYDPGDVFQHPHSIPVSLTAEDIARHGIPEQVAAEIRSAGLLS

SEQ ID NO: 11 - protein, 468 aa, Streptomyces albogriseolus
MDRYPRNAGTVVSPADPDYERARRIWNNRFDCRPRAIMYPRDEAEAAAALGYARDNEVPFRIRSGGHNSEGFCTVDDGIIIDLSKLGSIAVNNDRTRAVISPGVMLGEVYANLWSVGGTIPAGVCPDIRIGGHVLGGGIGMLVRSRGLLIDNLVGLTVVDARGERLQVDADHHPDLFWACKGGGGGNYGIATSYTFEMRPISDVTIFKLQWDWQGGMAVLDAWQHWLLSADSRVNARFNVFSSTVGTVMTVGLFEGSADELKSILQPLVEKFPPRDSFVRTMPYMECVGTFSEQVPSIRAKFVPALSAGPLDADALKTLERWHKDAPTGVKTGLYGLGGEVLGSVSPDSSAFAHRDASICVEYLGHWQGAEHDREHLGWLSGVREEMDAFMTGGAYVNSPDRDLENWLHAYYGESLPRLMDVKRRYDPDNVFAFEQSIPGSLTLPEARAAGLTDTVVEDLRARGLLSA

SEQ ID NO: 12 - protein, 468 aa, Streptomyces sp.
  source 1..468: note = Streptomyces sp. FXJ1.172
MGAYPDTIGTVVTPGDTRYERARRLWNERFDYRPRAVVLPGDSHETAAALRYVRERGIPFRIRSGGHNSEGFSGLDDGIVIDVSRLGALAVYGDRTRATLGPGVLLGEAYAALWALGGTIPAGVCPDIRIGGHVLGGGIGMLVRSRGLLIDHLVALTLIDARGEELTVDADHHPDLLWASRGGGGGNFGIVTSYTFAMRPIGDVTLFTLEWDARDGIAALDAWQHWLSQADPRVNSRFNLFPASADRALATGLFEGGERELTALLRPLTNACRPRATSVRALPYGDSIGTWTEQVPSVRAKFTPALAEGPLGAEALRTLGRRHEKAPPGVKTGLYGLGGAVLADVPPDHSAFAHRAAFLCVEYLGHWSDPGSDAEHLDWLSGTRAEMLPFMTGGAYVNSPDLDLENWLHAYYGDGLPRLMEVKRRYDPHGLFTFEQSIPTALTRAQARAARLPGPVVADLHARGLLTD

SEQ ID NO: 13 - protein, 468 aa, Streptomyces sp.
  source 1..468: note = Streptomyces sp. Ru87
MEEHPQHVGTVVTQADPDYEQARHIWNERFDFRPRAVFYPRNAEETAAAVTHVRDRGLPFRIRSGGHNSEGFCSVDDGVVIDLSKLASIGVNNDRTRATTGPGVLLGEVYATLAAVGGTIPAGVCPDIRIGGHVLGGGIGMLVRSRGLLIDSLVGLTLVDARGRILEVDDSSHPELMWACRGGGGGNYGIVTSYTFEMRPVSEVTLFTLEWDWNGGIGVLDTWQHWILRADTRVNARFAFLPGSTGTVMSTGLFEGPARELESILRPLVGKHPPRDSVVRTVPYAESVGTFTEPVPSIRAKFVPALAGGPLGTEALEILERRHRDAPAGVKTGLYGLGGAVLDNAPAKRSSFAHRDAPICVEYLGHWKDSAHDSEHLDWLTATREELHGFMTGGAYVNSPDRDLADWLHAYYGENLPGLMDVKRRYDPDDVFSFEQSIPTSLSPAEARAAGLTDTVVAELRGRGLLRS

SEQ ID NO: 14 - DNA, 1407 bp, Streptomyces albogriseolus
  source 1..1407: mol_type = genomic DNA
atggacaggtatccccggaacgccggcacggtggtctcgccggccgacccggactacgaacgggcacgacgcatctggaacaaccgcttcgactgccggccccgcgccatcatgtacccgcgcgacgaagcggaggcggcggccgcgctgggatacgcgcgggacaacgaggtcccgttccgtatccgctccggcggtcacaacagcgagggtttctgcacggtcgacgacggcatcatcatcgacctgtcgaagctcggctcgatcgccgtgaacaacgaccggacccgggccgtcatcagccccggtgtcatgctcggtgaggtgtacgcgaacctctggtccgtgggcggcaccatccccgccggggtctgcccggacatcaggatcggcgggcacgtgctcggcggcggcatcgggatgctcgtccgctcgcggggcctgctcatcgacaatctggtgggcctcaccgtggtcgacgcgcggggggagaggctccaggtcgacgcggaccaccacccggatctcttctgggcctgcaagggaggcggcggcggcaactacgggatcgccacctcgtacaccttcgagatgcggcccatctccgacgtgacgatcttcaagctgcagtgggactggcagggcggcatggcggtgctcgacgcctggcagcactggctcctctcggcggactcgcgggtcaacgcccggttcaacgtcttctccagtacggtcggtacggtcatgacggtcggcctgttcgagggttcggccgacgagctgaagtcgatcctccagccgctggtcgagaagttcccgccgcgtgactccttcgtacgcaccatgccctacatggagtgcgtggggacgttcagcgagcaagtgccctccatacgggccaagttcgtgcccgcgctgtcggcgggaccgctcgacgccgacgcactgaagaccctggagcgctggcacaaggacgcaccgaccggggtcaagacggggctctacgggctcggcggcgaggtgctcggcagtgtgtcgccggacagctcggcgttcgcccaccgcgacgcgtccatctgcgtcgagtacctcggccactggcagggggcggaacacgaccgggaacacctcggctggctgtccggcgtccgcgaggagatggacgccttcatgacgggcggagcctacgtcaacagtccggaccgcgatctggagaactggctgcacgcctactacggggagagcctcccgcggctgatggacgtgaagaggcggtacgaccccgacaacgtcttcgccttcgagcagagcatcccgggatccctcaccctcccggaggcccgcgccgcgggcctgaccgacacggtcgtcgaagacctgcgcgcccgcggacttctctccgcctga

SEQ ID NO: 15 - DNA, 1407 bp, Streptomyces sp.
  source 1..1407: mol_type = genomic DNA; note = Streptomyces sp. FXJ1.172
atgggggcgtatccggacaccatcggcacggtggtgacacccggcgacacgcgctacgaaagggcgcgccgcctgtggaacgagcgcttcgactaccggcccagggccgtcgtcctgccgggtgacagccacgagacggcagcggcgctccgctacgtccgggagcgcggcatcccgttccggatccgctccggcggccacaacagcgaggggttcagcggcctcgacgacggcatcgtcatcgacgtgtcgcgcctgggggcgctggcggtctacggtgaccgcacccgcgccaccctcggcccgggtgtcctgctcggcgaggcgtacgcggcgctgtgggcgctcggcggcacgatccccgcgggcgtctgccccgacatccgcatcggcggtcatgtcctgggcggcggcatcggcatgctggtacgttcccgcggcttgctcatcgaccacctggtcgcactcaccctgatcgacgcccgcggcgaggaactgacggtcgacgcggaccaccatccggacctgctctgggcgtcacgcggaggcggtggcgggaacttcgggatcgtcacctcctacaccttcgcgatgcgcccgatcggcgacgtgaccctgttcacgctggaatgggacgcgcgcgacggcatcgccgcactcgacgcctggcagcactggctcagccaggcggacccgcgggtgaactcccgcttcaacctgttccccgcctccgccgacagggccctggccaccggcctgttcgaagggggtgagcgtgaactcaccgccctgctccgaccgttgaccaacgcctgccggcctcgcgcgacatccgtacgcgccctgccgtacggggacagcatcggcacctggaccgagcaagtgccgtcggtacgcgccaagttcacccccgcactggccgaaggcccactcggcgccgaggcgctgcgcaccctgggccgccggcacgagaaggcgccgcccggcgtcaagacgggtctgtacggcctgggcggagcggtcctggccgacgtgccgcccgatcactccgcgttcgcgcaccgggcggccttcctgtgcgtggagtacctcggccactggagcgatcccggcagcgacgccgaacacctggactggctgtccggcacccgcgcggagatgctcccgttcatgaccggcggggcctacgtcaacagcccggacctcgatctggagaactggctgcacgcctactacggcgacggcctgccgcgcctcatggaggtcaagcgccgctacgacccgcacggcctcttcacgttcgagcagagcattcccaccgcgctcacccgggcccaggcccgtgccgcccggctccccggcccggtcgtcgccgacctgcacgcccgcggcctgctcaccgactga

SEQ ID NO: 16 - DNA, 1407 bp, Streptomyces sp.
  source 1..1407: mol_type = genomic DNA; note = Streptomyces sp. Ru87
atggaggaacacccgcagcacgtcggcactgtggtgacacaggccgatccggactacgagcaggcacggcacatctggaacgagcgtttcgacttccggccccgggcggtgttctacccccggaacgccgaggagacggcggccgccgtcacgcatgtacgggaccgggggctgccgttccgcatcaggtccggcggtcacaacagcgagggcttctgcagtgtggacgacggcgtcgtcatcgatctgtcgaagctggcgtcgataggcgtcaacaacgaccggacccgggcgacgaccggccccggtgtcctgctcggtgaggtctacgcgactctggccgccgtcggcggcaccatcccggccggcgtctgccccgacatccggatcggcgggcacgtgctcggcggcggcatcgggatgctcgtgcgctcccgcggcctgctcatcgacagcctggtggggctcaccctggtcgacgcccggggccggatcctggaggtcgacgactcctcgcacccggagctgatgtgggcctgccgtggcggcggcggcgggaactacggaatcgtcacgtcgtacaccttcgagatgcggccggtctccgaggtgacgctcttcacgctggagtgggactggaacggcggcatcggcgtcctcgacacctggcagcactggatcctccgggcggacacgcgcgtgaacgcccggttcgcgttcctgcccggctccaccggaacggtcatgtccaccgggctgttcgaggggccggcgcgggagctggagtcgatcctccggccgctggtcggaaagcacccgccgcgcgattccgtcgtccgcacggtgccgtacgcggagagcgtgggcacgttcaccgaaccggtcccctccatccgggccaagttcgtgcccgcgctcgccggcggcccgctcggcaccgaggccctggagatcctggaacgccggcaccgggacgcgccggccggcgtcaagacggggctgtacggactcggcggcgcggtgctcgacaacgcgcccgcgaagcgctcgtcgttcgcgcaccgggacgcgcccatctgcgtggagtacctcgggcactggaaggactccgcgcacgacagcgagcacctcgactggctgaccgcgacccgcgaggagctgcacggcttcatgaccggtggcgcctacgtcaacagcccggaccgcgacctcgccgactggctccacgcctactacggcgagaacctgcccggactcatggacgtcaagcgccgttacgacccggacgacgtgttctcgttcgagcagagcatccccacctccctctcccccgccgaggcccgcgccgcgggactgaccgacacggtcgtcgcggaactgcgcgggcgcggcctgctccgctcctga

SEQ ID NO: 17 - protein, 468 aa, unidentified
  source 1..468: note = Streptomyces lycii
MEEHPQHVGTVVTQADPDYEQARHIWNERFDFRPRAVFYPRNAEETAAAVTHVRDRGLPFRIRSGGHNSEGFCSVDDGVVIDLSKLASIGVNNDRTRATTGPGVLLGEVYATLAAVGGTIPAGVCPDIRIGGHVLGGGIGMLVRSRGLLIDSLVGLTLVDARGRILEVDDSSHPELMWACRGGGGGNYGIVTSYTFEMRPVSEVTLFTLEWDWNGGIGVLDTWQHWILRADTRVNARFAFLPGSTGTVMSTGLFEGPARELESILRPLVGKHPPRDSVVRTVPYAESVGTFTEPVPSIRAKFVPALAGGPLGTEALEILERRHRDAPAGVKTGLYGLGGAVLDNAPAKRSSFAHRDAPICVEYLGHWKDSAHDSEHLDWLTATREELHGFMTGGAYVNSPDRDLADWLHAYYGGNLPGLMDVKRRYDPDDVFSFEQSIPTSLSPAEARAAGLTDTVVAELRGRGLLRS

SEQ ID NO: 18 - protein, 470 aa, Frankia sp.
  source 1..470: note = Frankia sp. CiP3
MGTYPDTIGLVVTPSDTRYDRARSIWNERFDCRPQAVVYPRDAEETAAALCYVRDHGISFRIRSGGHNVEGFCTLDDGIVIDVSRLGSVAVNNDRSRAVIGPGVLLGEVYAVLWSVGGTIPAGVCPDIRIGGHVLGGGIGMLVRSRGLLIDSLVGLTVVDARGRILAVDETNHADLLWASRGGGGGNFGIVTSYTFKMRPISDVTLFTMKWDWADGIAVLDEWQRWILRADERVNVRFNLFPRSAGMAMTTGLFEGSADELKSIIKPLVDTHPPRETSMRTVPYMDSVGTWTQRVPSIRAKFVPALAARPLDSYAMKILDRWHRTAPAGVRTGFYGLGGAVLGDVPPEHSAFAHRNASICVEYLGHWDDAESDREHLDWLCGIRDEMLPFMTGGAYVNSPDRDLANWLHAYYGDRLPRLMDVKRRYDPHNVFSFEQSIPTSLTLPEAHAAGLPDAVIAELRSRNLLSEHA

SEQ ID NO: 19 - protein, 467 aa, Kutzneria sp.
  source 1..467: note = Kutzneria sp. CA-103260
MSRSQRVAATVTAADPGYDQARRIWNERFDCRPLAVLYPADVAEVAGAVRAARDDGVPFRIRSGGHNVEGFSVLDDGYVIDLSRLTGVTVDDDRTRGTVAAGTLLGDVYSGLWAAGVTIPAGVCPDIRIGGHVLGGGVGMLVRSRGMLVDSLVGLTMVDADGEVLEVNDDTHPDLMWACRGGGGGNFGVATSYTFHTRPIGDVTLVTAAWNFDNAITALDRWQHWAVEVDQRVNARFSLFSPGTGGGRMAVLFEGTSAEWESLARPLFEQCPPAEHTVRETAYINSVGQFSQRVPSIRAKFVPALAAGPLDAEALKILERHHRAAPDGVKTSFYGLGGAVLGADLAKRSAFAHRDALFCVEYLGHWHGAEHDAEQLGWLAGIRDELRPFMTGGAYVNSPDRDLTDWLHAYYGASLPRLMEVKRRYDPEDVFRFEQSIPVSLSPAAARAAGLTEAVVADLGARGLLTS

SEQ ID NO: 20 - DNA, 1422 bp, Streptomyces olivochromogenes
  source 1..1422: mol_type = genomic DNA
atgggaacacctgcaagatttacgggtacagttttaacgccgggggacgcgggctttgaagatgcatcgcggacttggaacgcttgttacgcgtcgcggccccgtgaagtcatggtttgccactcggcggaggcggtggccgaggcgcttggttctgctcgtggtcgtggtattcctttccgtgtgcggtccgggggccactccatggcggggctttcctccgtggcagacggagcgatcatcgacttgggcgggcttgatgccattaccctgagcgaagatagcagcctcgtgacggtcgggggcggcgcgcgcttgggtgatatttatcgtgaactctggaaggctggtgtgacggtgccggccggtacttgtccccgtatcggcatcggcggccacgtgttgggcggcggaatgggcgtactaagtcgtagccgtggtgcgctggtggatcatctggtgggactggaactggtgggcgcggacggccgcctgcgccaagtggacgacgacgaacatgcagatctgttttgggcttgccgcggcggcgggggaggcagcttcggaatcgcgacgagttacacgttccgcacgtcaccgattagcgaagttaccatttttacaattgcttggtcttgggcacagtttgcaccggctgttaaagcatggcagggttggctcgcgtcagcggaggacagaataaacaccttcctttctttattccctaggcagcaggatttgctggtggcgtttggtgtttttgatggtccggcggccgaattcgaagaattactccgtccgctggtcgcggcagcggagccgcaaacgctggtagttgaacccgtgccttttattcaggcggtcgatactgtggaggcgctgcagggcgaggcggcggcagccgatcaagtacgcgcgcaaggttcatcagcgatcgtcgccgagccgctgaacgaagccgcaatcgccaccctgcaggaattcttgaccgatccgccaagtcaccgcgccgaagtcgccgtttacggcatgggcggggccattggccatcgcgcccgcaccgataccgcatttgttcatcgcaccggtttaatggcctttgaatatcgcacagattgggatttaccagaggacgacggtaaaaatctggcgtgggtccgcgcactgcgacatagtatggccgaacataccaccggtgctgcttacgttaataccattgatctggcactggagaactggttgtgggcttattatgaagaaaatctgccgcgtctaatgcaagtaaaacgtcagtatgatccgggcgatgttttccagcatccgcactcgatcccagtaagcctgaccgccgaagatattgctcgtcacgggatcccagaacaggtcgccgccgaaattcgcagcgccggtctgctgtcttag

SEQ ID NO: 21 - DNA, 1407 bp, unidentified
  source 1..1407: mol_type = genomic DNA; note = Streptomyces lycii
atggaagaacacccacaacacgtaggaacagtggtgacgcaagcggacccggactatgaacaagcacgtcatatttggaacgaacgctttgattttcgtccgcgggcggtgttctatcctcgtaatgcggaagaaacggcggcggccgtgacgcatgttcgtgaccgggggcttccttttcgtattcgctcgggcgggcacaatagcgagggtttctgctctgttgatgacggtgtggtaatcgatttgtcgaagttagcatcgatcggcgtgaataacgaccgtacccgtgctactactggtccgggggtgttgctcggtgaggtgtacgccacgttggcggcggtgggtggtaccatcccggccggcgtgtgtcctgatattcgcatcggcggccacgtgttgggagggggcatcgggatgctggttcgttcgcgcgggttgctgattgattccctggtggggctgacgctggtggatgctcgcggccgcattctggaggtggacgatagtagccatcccgaactaatgtgggcatgtcgcggcggaggaggcggcaactatggtattgttacctcttatacttttgaaatgcgtccagttagcgaagtaacactgttcactttagaatgggactggaacggcggaatcggcgtcctggacacttggcagcactggatccttcgtgccgatacacgcgttaatgctagatttgccttccttcctggttccacgggcacggtcatgtccacgggactgttcgaaggtccagcacgcgagttggaatcgatattacgcccgctggtcggcaaacacccgccgcgggatagtgtagttaggaccgtcccgtacgctgaatccgtcggcacctttaccgaaccggtcccgtcaattcgtgccaaattcgttccggcgctggcgggcggcccgctcggtacagaagccctggagattctggaacgtcgccaccgcgacgcgccggcaggcgtcaaaaccggcctgtatggcctgggcggagcggtcctcgacaacgcgcccgcgaaacgtagttcattcgcgcatcgcgacgccccgatttgcgtcgaatatttaggtcattggaaggatagcgcccatgattctgaacatttagattggctgaccgcaacccgggaggagcttcacggttttatgaccggtggcgcgtacgtgaatagccctgatcgtgatctggctgattggctccatgcatattacggtggtaacctgccgggtctgatggatgttaaacgtcgatacgatccagatgatgtttttagttttgaacagtcaatcccaacctctctatcacccgcagaggcccgcgcagccggtctgaccgataccgtagtagctgagttacgcggtcgcggtctgctgcgaagctag

SEQ ID NO: 22 - DNA, 1413 bp, Frankia sp.
  source 1..1413: mol_type = genomic DNA; note = Frankia sp. CiP3
atgggaacataccctgatacaataggtctggttgttaccccgagcgatacgcggtacgaccgggcacggtcgatctggaatgaacgttttgattgtcgtcctcaggcagtcgtgtatcctcgggacgcggaggaaacggctgctgccttatgttatgtccgtgatcatgggatcagttttcgcattcgttcgggcgggcacaatgtggaggggttctgcacgcttgacgacgggatcgtaattgatgtttctcgcttaggtagcgtggccgtcaataatgaccgctcccgcgcggtgatcggtccgggggtgttgctgggtgaggtgtacgcggtgctttggtccgtgggcggtaccatcccggccggagtgtgcccggatattcgtatcggcggccatgtgttgggcgggggcattggtatgctggtgcgctcccgaggccttcttatcgatagcctggttggtctgaccgtcgttgatgcccgtggtcgtattctggccgttgatgagacaaaccacgctgacttgttgtgggcgtcccgcggcggcggaggcggcaatttcggaatcgtgactagctatacatttaagatgcgtcccattagcgatgttaccctgttcactatgaaatgggattgggcggacggaatcgcggtgttggacgaatggcaacgctggattttacgtgcagatgaacgagttaatgttaggttcaacctgtttccacgtagcgccggtatggcaatgacgaccggcctgttcgaaggctcagccgatgaactcaagagtattattaaaccgctggtagatacccacccgccgcgcgagacatcgatgcgtaccgtcccctatatggattcagtgggcacctggacccagcgtgtcccatctattcgtgcgaaatttgtacccgccttagctgctcgcccactggattcttatgcaatgaaaattttagatcgctggcatcgtactgcgccggcaggcgtccgcacgggcttctacggcctaggaggcgcggtactgggggatgttccgccagaacactcggcgtttgcacatcgcaacgcgtcaatctgtgtcgaatatttaggtcattgggacgacgcggaaagtgaccgcgaacatctggattggctgtgcggtattcgtgatgaaatgctcccttttatgaccggtggcgcgtatgtcaactctccggacagagatctagctaactggctgcatgcctactatggtgatcgtctgcccagactcatggatgtgaaacgccgctacgacccgcacaacgtatttagttttgaacagtcgataccaacctcactgacgctgccggaggcgcacgcagcaggtctgccggacgcggtaatcgccgaactccgcagtcgcaacctgctgagcgaacatgcctag

SEQ ID NO: 23 - DNA, 1404 bp, Kutzneria sp.
  source 1..1404: mol_type = genomic DNA; note = Kutzneria sp. CA-103260
atgagtaggagtcaaagagtggccgcaactgtgacggcggcagatcctggatatgatcaagctcgtcgtatttggaacgaacgttttgattgccggccgctcgcggtgctttacccggcggacgtggcggaagtagcgggagcggtcagagcagctcgggacgacggggtcccgtttcgtattcgttcgggcgggcacaatgttgaaggttttagcgttctcgacgacggctacgttattgacttatcgcgccttacgggggtcacggttgatgatgatcgtacgcgcgggacggtcgcggcggggacgttactgggggacgtctattctggtctgtgggcggcgggcgtgaccatcccggcgggggtgtgccctgatatacgtatcggcggccacgtgttgggcggaggcgtgggcatgcttgtgcgttcgcgcggcatgttggtggattctttagtgggccttaccatggtggacgcggacggcgaggtgttggaggtgaacgacgatacacatcctgatctgatgtgggcttgccgcggaggaggcggcggcaattttggtgttgctactagctatacatttcacacgcggccgattggcgacgtgaccctagtgaccgcggcgtggaactttgataatgctattaccgcgttggatcgttggcagcattgggctgttgaagtagaccagcgcgtcaatgcacgtttctccttgttctcccctggtaccgggggcggccgtatggcggtcctgttcgagggtacttcagccgaatgggaaagcctggcgcgcccgctattcgagcagtgtccgcccgcggagcacaccgtccgagaaactgcttatattaacagcgttggtcaattctcccagcgagtcccgtcaatccgtgcaaaatttgttccggcgctggcggcaggcccgctggacgccgaggctttaaaaattctggaacgtcatcaccgcgccgccccggatggcgtaaagacctcattttacggcctgggaggcgccgtactgggtgccgatctggccaaacgtagcgctttcgcacatcgagatgccctgttctgtgttgaatatctcggtcattggcatggtgccgagcacgatgccgaacaactgggttggctggcaggtatccgcgatgaactgcgcccatttatgaccggtggtgcctatgttaactcgccggatcgggatctgaccgattggttgcatgcatactatggtgctagcctgccccgcctgatggaagtaaaacgccgctacgatccagaagatgtttttcgcttcgaacagagtatcccagtaagtctgagcccagccgccgcacgcgccgcaggtctgacagaagcagtcgtagccgatctcggtgcccgcggtttactgacctcttag

SEQ ID NO: 24 - protein, 506 aa, Streptomyces sp.
  source 1..506: note = Streptomyces sp. CNH-287
MTADPSSERSDMNEADEVNEVDELSETGQTSGTKGKRPFTGRVIGPADGEFDEARRVWNECFAARPKEIVYCADTRDVVRALREVRQRGGPFRVRSGGHSMSGLSVLDDGTVLDVSGLDDIQVSEDASTVTVGSGAHLGDIFRALWARGVTVPAGFCPEIGIAGHVLGGGAGILVRSRGFLSDHLVALEMVDSEGRIVVADHDSHHELLWASRGGGGGNFGIATSFTLRTQPIGDVTLFTIAWDWDRGAEAIKAWQEWLATADGRINTLFIAYPQDQDMFAALGCFEGDAAELEPLIAPLVHAVEPTEKVAETMPWIEALSFVETMQGEAMKATSVRAKGNLSFVTEPLGDRAVEEIKKALAQAPSHRAEVVLYGLGGAVAAKGRRETAFVHRDAPVALNYHTDWDDEAEDDLNFAWIQNLRASVAAHTEGRGSYVNTIDLTVEHWLWDYYEENLPRLMAVKKRYDPEDVFRHPQSIPVSLTEAEAAELGIPPHIAEELRAARQLR

SEQ ID NO: 25 - protein, 519 aa, synthetic construct
MANPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

(19) United States
(12) Patent Application Publication
(10) Pub. No.: US 2025/0290048 A1
(43) Pub. Date: Sep. 18, 2025

(54) FLAVIN-DEPENDENT OXIDASES HAVING CANNABINOID SYNTHASE ACTIVITY

(71) Applicant: GENOMATICA, INC., San Diego, CA (US)

(72) Inventors: Jamison Parker HUDDLESTON, San Diego, CA (US); Andreas SCHIRMER, San Diego, CA (US); Trevor Nelson PURDY, San Diego, CA (US)

(21) Appl. No.: 18/687,169

(22) PCT Filed: Aug. 31, 2022
(86) PCT No.: PCT/US2022/075759

§ 371 Date: Feb. 27, 2024

Related U.S. Application Data
(60) Provisional application No. 63/239,704, filed on Sep. 1, 2021.
(60) Provisional application No. 63/315,866, filed on Mar. 2, 2022.

(51) Int. Cl. C12N 9/02; C12N 1/20; C12N 9/10; C12N 9/88; C12N 15/70; C12P 17/06; C12R 1/19

(52) U.S. Cl. CPC C12N 9/0004; C12N 1/20; C12N 9/1029; C12N 9/1085; C12N 9/88; C12N 15/70; C12P 17/06; C12R 2001/19; C12Y 121/03007; C12Y 203/01206; C12Y 205/01001; C12Y 404/01026

(57) ABSTRACT

The disclosure relates to a non-natural flavin-dependent oxidase comprising at least one amino acid variation as compared to a wild type flavin-dependent oxidase, wherein the non-natural flavin-dependent oxidase does not comprise a disulfide bond, and wherein the non-natural flavin-dependent oxidase is capable of oxidative cyclization of a prenylated aromatic compound into a cannabinoid. The disclosure also relates to a nucleic acid, an expression construct, and an engineered cell for making the non-natural flavin-dependent oxidase. Also provided are compositions comprising the non-natural flavin-dependent oxidase; isolated non-natural flavin-dependent oxidase and methods of making the same; cell extracts comprising the non-natural flavin-dependent oxidase; methods of making cannabinoids; and compositions comprising a cannabinoid and/or the non-natural flavin-dependent oxidase.

### SEQUENCE LISTING

The instant application contains a Sequence Listing which has been submitted electronically in XML format and is hereby incorporated by reference in its entirety. Said Sequence Listing XML, created on Oct. 11, 2024, is named “0311-0005US1.xml” and is 45,676 bytes in size. The sequence listing contained in this .XML file is part of the specification and is hereby incorporated by reference herein in its entirety.

### FIELD OF THE INVENTION

The disclosure relates to a non-natural flavin-dependent oxidase comprising at least one amino acid variation as compared to a wild type flavin-dependent oxidase, wherein the non-natural flavin-dependent oxidase does not comprise a disulfide bond, and wherein the non-natural flavin-dependent oxidase is capable of oxidative cyclization of a prenylated aromatic compound into a cannabinoid. The disclosure also relates to a nucleic acid, an expression construct, and an engineered cell for making the non-natural flavin-dependent oxidase. Also provided are compositions comprising the non-natural flavin-dependent oxidase; isolated non-natural flavin-dependent oxidase and methods of making the same; cell extracts comprising the non-natural flavin-dependent oxidase; methods of making cannabinoids; and compositions comprising a cannabinoid and/or the non-natural flavin-dependent oxidase.

### BACKGROUND

Cannabinoids constitute a varied class of chemicals, typically prenylated polyketides derived from fatty acid and isoprenoid precursors, that bind to cellular cannabinoid receptors. Modulation of these receptors has been associated with different types of physiological processes including pain-sensation, memory, mood, and appetite. Endocannabinoids, which occur in the body, phytocannabinoids, which are found in plants such as cannabis, and synthetic cannabinoids, can have activity on cannabinoid receptors and elicit biological responses. Recently, cannabinoids have drawn significant scientific interest in their potential to treat a wide array of disorders, including insomnia, chronic pain, epilepsy, and post-traumatic stress disorder (Babson et al. (2017), Curr Psychiatry Rep 19:23; Romero-Sandoval et al. (2017) Curr Rheumatol Rep 19:67; O'Connell et al. (2017) Epilepsy Behav 70:341-348; Zir-Aviv et al. (2016) Behav Pharmacol 27:561-569). The use of cannabinoids as therapeutics requires their production in large quantities and at high purity. However, purifying individual cannabinoid compounds from C. sativa can be time-consuming and costly, and it can be difficult to isolate a pure sample of a compound of interest. Thus, engineered cells can be a useful alternative for the production of a specific cannabinoid or cannabinoid precursor.

### SUMMARY OF THE INVENTION

The present disclosure relates to flavin-dependent oxidases that have cannabinoid synthase activity.

In some embodiments, the disclosure provides a non-natural flavin-dependent oxidase comprising at least one amino acid variation as compared to a wild type flavin-dependent oxidase, wherein the non-natural flavin-dependent oxidase: (i) does not comprise a disulfide bond, (ii) is capable of oxidative cyclization of a prenylated aromatic compound into a cannabinoid, and (iii) comprises at least 70% sequence identity to any one of SEQ ID NOs:1-3, any one of SEQ ID NOs:10-13, or any one of SEQ ID NOs:17-19.

In some embodiments, the prenylated aromatic compound is cannabigerolic acid (CBGA), cannabigerorcinic acid (CBGOA), cannabigerivarinic acid (CBGVA), cannabigerorcinol (CBGO), cannabigerivarinol (CBGV), or cannabigerol (CBG). In some embodiments, the non-natural flavin-dependent oxidase is not glycosylated. In some embodiments, the non-natural flavin-dependent oxidase comprises a monovalently bound FAD cofactor. In some embodiments, the non-natural flavin-dependent oxidase comprises a bivalently bound FAD cofactor.

In some embodiments, the non-natural flavin-dependent oxidase is capable of oxidative cyclization of a prenylated aromatic compound into a cannabinoid at about pH 7.5. In some embodiments, catalytic activity of the non-natural flavin-dependent oxidase is substantially the same from about pH 5 to about pH 8.

In some embodiments, the non-natural flavin-dependent oxidase comprises at least 80% sequence identity to any one of SEQ ID NOs:1-3, any one of SEQ ID NOs:10-13, or any one of SEQ ID NOs:17-19. In some embodiments, the non-natural flavin-dependent oxidase comprises at least 90% sequence identity to any one of SEQ ID NOs:1-3, any one of SEQ ID NOs:10-13, or any one of SEQ ID NOs:17-19. In some embodiments, the non-natural flavin-dependent oxidase comprises at least 90% sequence identity to any one of SEQ ID NOs:1-3, SEQ ID NO:10, SEQ ID NO:11, SEQ ID NO:13, or SEQ ID NO:19.

In some embodiments, the non-natural flavin-dependent oxidase converts CBGA to cannabichromenic acid (CBCA). In some embodiments, the non-natural flavin-dependent oxidase converts CBGA to cannabidiolic acid (CBDA). In some embodiments, the non-natural flavin-dependent oxidase converts CBGA to Δ9-tetrahydrocannabinolic acid (THCA). In some embodiments, the non-natural flavin-dependent oxidase converts CBGA to CBCA, CBDA, and/or THCA at about pH 4 to about pH 9. In some embodiments, the non-natural flavin-dependent oxidase converts CBGA to CBCA.

In some embodiments, the non-natural flavin-dependent oxidase further comprises an affinity tag, a purification tag, a solubility tag, or a combination thereof.

In some embodiments, the disclosure provides to a polynucleotide comprising a nucleic acid sequence encoding the non-natural flavin-dependent oxidase described herein. In some embodiments, the disclosure provides a polynucleotide comprising: (a) a nucleic acid sequence encoding a polypeptide having at least 80% sequence identity to any one of SEQ ID NOs:1-3, any one of SEQ ID NOs:10-13, or any one of SEQ ID NOs:17-19; and (b) a heterologous regulatory element operably linked to the nucleic acid sequence. In some embodiments, the polypeptide comprises at least 90% sequence identity to any one of SEQ ID NOs:1-3, SEQ ID NO:10, SEQ ID NO:11, SEQ ID NO:13, or SEQ ID NO:19. In some embodiments, the disclosure provides an expression construct comprising the polynucleotide described herein.

In some embodiments, the disclosure provides an engineered cell comprising the non-natural flavin-dependent oxidase, the polynucleotide, the expression construct, or a combination thereof.

In some embodiments, the engineered cell further comprises a cannabinoid biosynthesis pathway enzyme. In some embodiments, the cannabinoid biosynthesis pathway enzyme comprises olivetol synthase (OLS), olivetolic acid cyclase (OAC), prenyltransferase, or a combination thereof. In some embodiments, the cell is a bacterial cell. In some embodiments, the cell is an E. coli cell.

In some embodiments, the disclosure provides a cell extract or cell culture medium comprising cannabigerolic acid (CBGA), cannabichromenic acid (CBCA), tetrahydrocannabinolic acid (THCA), cannabidiolic acid (CBDA), cannabigerol (CBG), cannabichromene (CBC), cannabidiol (CBD), tetrahydrocannabinol (THC), cannabigerorcinic acid (CBGOA), cannabiorcichromenic acid (CBCOA), cannabidiorcinic acid (CBDOA), tetrahydrocannabiorcinic acid (THCOA), cannabigerivarinic acid (CBGVA), cannabichromevarinic acid (CBCVA), cannabidivarinic acid (CBDVA), tetrahydrocannabivarinic acid (THCVA), cannabigerorcinol (CBGO), cannabichromeorcin (CBCO), cannabidiorcin (CBDO), tetrahydrocannabiorcin (THCO), cannabigerivarinol (CBGV), cannabichromevarin (CBCV), cannabidivarin (CBDV), tetrahydrocannabivarin (THCV), an isomer, analog or derivative thereof, or a combination thereof derived from the engineered cell described herein. In some embodiments, the cell extract or cell culture medium comprises CBGA and CBCA.

In some embodiments, the disclosure provides a method of making a cannabinoid selected from CBDA, CBD, CBDOA, CBDVA, CBDO, CBDV, THCA, THC, THCOA, THCVA, THCO, THCV, CBCA, CBC, CBCOA, CBCVA, CBCO, CBCV, an isomer, analog or derivative thereof, or a combination thereof, comprising: culturing the engineered cell described herein, isolating the cannabinoid from the cell extract or cell culture medium described herein, or both. In some embodiments, the method makes CBCA, CBC, CBCOA, CBCVA, CBCO, CBCV, an isomer, analog or derivative thereof. In some embodiments, the method makes CBCA.

In some embodiments, the disclosure provides a method of making CBCA, CBDA, THCA, or an isomer, analog or derivative thereof, comprising contacting CBGA with the non-natural flavin-dependent oxidase described herein. In some embodiments, the CBGA is contacted with a flavin-dependent oxidase comprising any one of SEQ ID NOs:1-3, any one of SEQ ID NOs:10-13, or any one of SEQ ID NOs:17-19. In some embodiments, the CBGA is contacted with a flavin-dependent oxidase comprises any one of SEQ ID NOs:1-3, SEQ ID NO:10, SEQ ID NO:11, SEQ ID NO:13, or SEQ ID NO:19. In some embodiments, the method makes CBCA. In some embodiments, the contacting occurs at about pH 4 to about pH 9. In some embodiments, the method is performed in an in vitro reaction medium. In some embodiments, the in vitro reaction medium comprises a surfactant. In some embodiments, the surfactant is about 0.01% (v/v) to about 1% (v/v) of the in vitro reaction medium. In some embodiments, the surfactant is 2-[4-(2,4,4-trimethylpentan-2-yl)phenoxy]ethanol (Triton™ X-100). In some embodiments, the in vitro reaction medium comprises about 0.1% (v/v) 2-[4-(2,4,4-trimethylpentan-2-yl)phenoxy]ethanol.

In some embodiments, the disclosure provides a method of making an isolated non-natural flavin-dependent oxidase, comprising isolating the non-natural flavin-dependent oxidase expressed in the engineered cell described herein. In some embodiments, the disclosure provides an isolated non-natural flavin-dependent oxidase made by the method described herein.

In some embodiments, the disclosure provides a composition comprising a cannabinoid or an isomer, analog or derivative thereof obtained from the engineered cell described herein, the cell extract described herein, or the method described herein. In some embodiments, the cannabinoid is THCA, THCOA, THCVA, THC, THCO, THCV, CBDA, CBDOA, CBDVA, CBD, CBDO, CBDV, CBCA, CBCOA, CBCVA, CBC, CBCO, CBCV, or an isomer, analog or derivative thereof, or a combination thereof. In some embodiments, the cannabinoid is CBCA. In some embodiments, the cannabinoid is 50% or greater, 60% or greater, 70% or greater, 80% or greater, 85% or greater, 90% or greater, 91% or greater, 92% or greater, 93% or greater, 94% or greater, 95% or greater, 96% or greater, 97% or greater, 98% or greater, 99% or greater, 99.2% or greater, 99.4% or greater, 99.5% or greater, 99.6% or greater, 99.7% or greater, 99.8% or greater, or 99.9% or greater of total cannabinoid compound(s) in the composition.

In some embodiments, the composition is a therapeutic or medicinal composition. In some embodiments, the composition is a topical composition. In some embodiments, the composition is an edible composition.

In some embodiments, the disclosure provides a composition comprising: (a) the flavin-dependent oxidase described herein; and (b) a cannabinoid, the prenylated aromatic compound, or both. In some embodiments, the flavin-dependent oxidase comprises any one of SEQ ID NOs:1-3, any one of SEQ ID NOs:10-13, or any one of SEQ ID NOs:17-19. In some embodiments, the flavin-dependent oxidase comprises any one of SEQ ID NOs:1-3, SEQ ID NO:10, SEQ ID NO:11, SEQ ID NO:13, or SEQ ID NO; 19. In some embodiments, the cannabinoid or prenylated aromatic compound is CBGA, CBGOA, CBGVA, CBG, CBGO, CBGV, THCA, THCOA, THCVA, THCO, THC, THCV, CBDA, CBDOA, CBDVA, CBD, CBDO, CBDV, CBCA, CBCOA, CBCVA, CBC, CBCO, CBCV, or an isomer, analog, or derivative thereof, or a combination thereof. In some embodiments, the composition further comprises an enzyme in a cannabinoid biosynthesis pathway. In some embodiments, the cannabinoid biosynthesis pathway enzyme comprises olivetol synthase (OLS), olivetolic acid cyclase (OAC), an enzyme in a geranyl pyrophosphate (GPP) pathway, prenyltransferase, or a combination thereof.

### BRIEF DESCRIPTION OF THE DRAWINGS

The following drawings form part of the present specification and are included to further demonstrate exemplary embodiments of certain aspects of the present disclosure.

FIG. 1 shows the structure of cannabigerolic acid (CBGA), cannabigerivarinic acid (CBGVA), cannabigerorcinic acid (CBGOA), two regioisomers of cannabichromenic acid (CBCA) denoted as CBCA-A and CBCA-B, cannabichromevarinic acid (CBCVA), and cannabiorcichromenic acid (CBCOA).

FIG. 2 shows the R- and S-enantiomers of CBCA-A, as described in embodiments herein.

FIG. 3A shows an exemplary HPLC chromatogram of a racemic CBCA-A standard, which comprises the enantiomers depicted in FIG. 2.

FIG. 3B shows an exemplary HPLC chromatogram of the CBCA-A products formed by an in vitro reaction of Psx9 using CBGA as a substrate at pH 7.4, as described in Example 3 herein.

FIG. 4 shows an exemplary LC/MS spectra of cannabinoid standards and the reaction products of Sox9 with CBGA. The top and middle panels of FIG. 4 show the LC/MS spectra of a CBDA standard and a CBCA-A standard, respectively. The bottom panel of FIG. 4 shows the LC/MS spectra of the reaction products of Sox9 with CBGA.

FIG. 5 shows a sequence alignment of the enzymes THCAS (SEQ ID NO:25), ThdO (SEQ ID NO:11), Kcx9 (SEQ ID NO:19), Clz9 (SEQ ID NO:24), Psx9 (SEQ ID NO:2), Tcz9 (SEQ ID NO:1), Svx9 (SEQ ID NO:3), and Sox9 (SEQ ID NO:10), as described in embodiments herein. Asterisk symbols (*) indicate the amino acid positions that have a single, fully conserved residue. Colon symbols (:) indicate conservation between amino acid groups of highly similar properties. Period symbols (.) indicate conservation between amino acid groups of weakly similar properties.

### DETAILED DESCRIPTION OF THE INVENTION

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” means that the specific terms recited are representative examples and embodiments of the disclosure that are not intended to be limited to the specific examples referenced or cited unless explicitly stated otherwise.

As used herein, “about” can mean plus or minus 10% of the provided value. Where ranges are provided, they are inclusive of the boundary values. “About” can additionally or alternately mean either within 10% of the stated value, or within 5% of the stated value, or in some cases within 2.5% of the stated value; or, “about” can mean rounded to the nearest significant digit.

As used herein, “between” is a range inclusive of the ends of the range. For example, a number between x and y explicitly includes the numbers x and y, and any numbers that fall within x and y.

A “nucleic acid,” “nucleic acid molecule,” “nucleic acid sequence,” “nucleotide sequence,” “oligonucleotide,” or “polynucleotide” means a polymeric compound including covalently linked nucleotides. The term “nucleic acid” includes ribonucleic acid (RNA) and deoxyribonucleic acid (DNA), both of which may be single- or double-stranded. DNA includes, but is not limited to, complementary DNA (cDNA), genomic DNA, plasmid or vector DNA, and synthetic DNA. In some embodiments, the disclosure provides a nucleic acid encoding any one of the polypeptides disclosed herein, e.g., is directed to a polynucleotide encoding a flavin-dependent oxidase or a variant thereof.

A “gene” refers to an assembly of nucleotides that encode a polypeptide and includes cDNA and genomic DNA nucleic acid molecules. In some embodiments, “gene” also refers to a non-coding nucleic acid fragment that can act as a regulatory sequence preceding (i.e., 5′) and following (i.e., 3′) the coding sequence.

As used herein, the term “operably linked” means that a polynucleotide of interest, e.g., the polynucleotide encoding a nuclease, is linked to the regulatory element in a manner that allows for expression of the polynucleotide. In some embodiments, the regulatory element is a promoter. In some embodiments, a nucleic acid expressing the polypeptide of interest is operably linked to a promoter on an expression vector.

As used herein, “promoter,” “promoter sequence,” or “promoter region” refers to a DNA regulatory region or polynucleotide capable of binding RNA polymerase and involved in initiating transcription of a downstream coding or non-coding sequence. In some embodiments, the promoter sequence includes the transcription initiation site and extends upstream to include the minimum number of bases or elements used to initiate transcription at levels detectable above background. In some embodiments, the promoter sequence includes a transcription initiation site, as well as protein binding domains responsible for the binding of RNA polymerase. Eukaryotic promoters typically contain “TATA” boxes and “CAT” boxes. Various promoters, including inducible promoters, may be used to drive expression of the various vectors of the present disclosure.

An “expression vector” or vectors (“an expression construct”) can be constructed to include one or more protein of interest-encoding nucleic acids (e.g., nucleic acid encoding a THCAS described herein) operably linked to expression control sequences functional in the host organism. Expression vectors applicable for use in the microbial host organisms provided include, for example, baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, herpes simplex virus, and the like), P1-based artificial chromosomes, yeast plasmids, yeast artificial chromosomes, and any other vectors specific for specific hosts of interest (such as E. coli and yeast). In some embodiments, the expression vector comprises a nucleic acid encoding a protein described herein, e.g., a flavin-dependent oxidase.

Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like. When two or more exogenous encoding nucleic acids (e.g., a gene encoding a flavin-dependent oxidase and an additional gene encoding another enzyme in a cannabinoid biosynthesis pathway such as, e.g., OLS, OAC, prenyltransferase, and/or an enzyme in the GPP pathway as described herein) are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein. The following vectors are provided by way of example; for bacterial host cells: pQE vectors (Qiagen), pBluescript plasmids, pNH vectors, lambda-ZAP vectors (Stratagene); pTrc99a, pKK223-3, pDR540, and pRIT2T (Pharmacia); for eukaryotic host cells: pXT1, pSG5 (Stratagene), pSVK3, pBPV, pMSG, and pSVLSV40 (Pharmacia). However, any other plasmid or other vector may be used so long as it is compatible with the host cell.

The term “host cell” refers to a cell into which a recombinant expression vector has been introduced, or “host cell” may also refer to the progeny of such a cell. Because modifications may occur in succeeding generations, for example, due to mutation or environmental influences, the progeny may not be identical to the parent cell, but are still included within the scope of the term “host cell.” In some embodiments, the present disclosure provides a host cell comprising an expression vector that comprises a nucleic acid encoding a flavin-dependent oxidase or variant thereof. In some embodiments, the host cell is a bacterial cell, a fungal cell, an algal cell, a cyanobacterial cell, or a plant cell.

A genetic alteration that makes an organism or cell non-natural can include, for example, modifications introducing expressible nucleic acids encoding metabolic polypeptides, other nucleic acid additions, nucleic acid deletions and/or other functional disruption of the organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon.

A host cell, organism, or microorganism engineered to express or overexpress a gene, a nucleic acid, nucleic acid sequence, or nucleic acid molecule, or to overexpress an enzyme or polypeptide has been genetically engineered through recombinant DNA technology to include a gene or nucleic acid sequence that it does not naturally include that encodes the enzyme or polypeptide or to express an endogenous gene at a level that exceeds its level of expression in a non-altered cell. As non-limiting examples, a host cell, organism, or microorganism engineered to express or overexpress a gene, a nucleic acid, nucleic acid sequence, or nucleic acid molecule, or to overexpress an enzyme or polypeptide can have any modifications that affect a coding sequence of a gene, the position of a gene on a chromosome or episome, or regulatory elements associated with a gene. A gene can also be overexpressed by increasing the copy number of a gene in the cell or organism. In some embodiments, overexpression of an endogenous gene comprises replacing the native promoter of the gene with a constitutive promoter that increases expression of the gene relative to expression in a control cell with the native promoter. In some embodiments, the constitutive promoter is heterologous.

Similarly, a host cell, organism, or microorganism engineered to under-express (or to have reduced expression of) a gene, nucleic acid, nucleic acid sequence, or nucleic acid molecule, or to under-express an enzyme or polypeptide can have any modifications that affect a coding sequence of a gene, the position of a gene on a chromosome or episome, or regulatory elements associated with a gene. Specifically included are gene disruptions, which include any insertions, deletions, or sequence mutations into or of the gene or a portion of the gene that affect its expression or the activity of the encoded polypeptide. Gene disruptions include “knockout” mutations that eliminate expression of the gene. Modifications to under-express or down-regulate a gene also include modifications to regulatory regions of the gene that can reduce its expression.

The term “exogenous” is intended to mean that the referenced molecule or the referenced activity is introduced into the host cell or host organism. The molecule can be introduced, for example, by introduction of an encoding nucleic acid into the host genetic material such as by integration into a host chromosome or as non-chromosomal genetic material that may be introduced on a vehicle such as a plasmid. The term “exogenous nucleic acid” means a nucleic acid that is not naturally-occurring within the host cell or host organism. Exogenous nucleic acids may be derived from or identical to a naturally-occurring nucleic acid or it may be a heterologous nucleic acid. For example, a non-natural duplication of a naturally-occurring gene is considered to be an exogenous nucleic acid sequence. An exogenous nucleic acid can be introduced in an expressible form into the host cell or host organism. The term “exogenous activity” refers to an activity that is introduced into the host cell or host organism. The source can be, for example, a homologous or heterologous encoding nucleic acid that expresses the referenced activity following introduction into the host cell or host organism.

Accordingly, the term “endogenous” refers to a referenced molecule or activity that is naturally present in the host cell or host organism. Similarly, the term when used in reference to expression of an encoding nucleic acid refers to expression of an encoding nucleic acid contained within the host cell or host organism.

The term “heterologous” refers to a molecule or activity derived from a source other than the referenced species, whereas “homologous” refers to a molecule or activity derived from the host microbial organism/species. Accordingly, exogenous expression of an encoding nucleic acid can utilize either or both of a heterologous or homologous encoding nucleic acid.

When used to refer to a genetic regulatory element, such as a promoter, operably linked to a gene, the term “homologous” refers to a regulatory element that is naturally operably linked to the referenced gene. In contrast, a “heterologous” regulatory element is not naturally found operably linked to the referenced gene, regardless of whether the regulatory element is naturally found in the host cell or host organism.

It is understood that more than one exogenous nucleic acid(s) can be introduced into the host cell or host organism on separate nucleic acid molecules, on polycistronic nucleic acid molecules, or a combination thereof, and still be considered as more than one exogenous nucleic acid. For example, as disclosed herein, a host cell or host organism can be engineered to express at least two, three, four, five, six, seven, eight, nine, ten or more exogenous nucleic acids encoding a desired pathway enzyme or protein. In the case where two or more exogenous nucleic acids encoding a desired activity are introduced into a host cell or host organism, it is understood that the two or more exogenous nucleic acids can be introduced as a single nucleic acid, for example, on a single plasmid, on separate plasmids, can be integrated into the host chromosome at a single site or multiple sites, and still be considered as two or more exogenous nucleic acids. Similarly, it is understood that more than two exogenous nucleic acids can be introduced into a host cell or host organism in any desired combination, for example, on a single plasmid, on separate plasmids, can be integrated into the host chromosome at a single site or multiple sites, and still be considered as two or more exogenous nucleic acids, for example three exogenous nucleic acids. Thus, the number of referenced exogenous nucleic acids or biosynthetic activities refers to the number of encoding nucleic acids or the number of biosynthetic activities, not the number of separate nucleic acids introduced into the host cell or host organism.

Genes or nucleic acid sequences can be introduced stably or transiently into a host cell host cell or host organism using techniques well known in the art including, but not limited to, conjugation, electroporation, chemical transformation, transduction, transfection, and ultrasound transformation. Optionally, for exogenous expression in E. coli or other prokaryotic host cells, some nucleic acid sequences in the genes or cDNAs of eukaryotic nucleic acids can encode targeting signals such as an N-terminal mitochondrial or other targeting signal, which can be removed before transformation into the prokaryotic host cells, if desired. For example, removal of a mitochondrial leader sequence led to increased expression in E. coli (Hoffmeister et al. (2005), J Biol Chem 280: 4329-4338). For exogenous expression in yeast or other eukaryotic host cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques known in the art to achieve optimized expression of the proteins.

In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Computer algorithms for codon optimizing a particular sequence for expression in a particular host cell are available and include, e.g., Integrated DNA Technologies' Codon Optimization tool, Entelechon's Codon Usage Table Analysis Tool, GenScript's OptimumGene tool, and the like. In some embodiments, the disclosure provides codon optimized polynucleotides expressing a flavin-dependent oxidase or variant thereof.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The start of the protein or polypeptide is known as the “N-terminus” (and also referred to as the amino-terminus, NH₂-terminus, N-terminal end or amine-terminus), referring to the free amine (—NH₂) group of the first amino acid residue of the protein or polypeptide. The end of the protein or polypeptide is known as the “C-terminus” (and also referred to as the carboxy-terminus, carboxyl-terminus, C-terminal end, or COOH-terminus), referring to the free carboxyl group (—COOH) of the last amino acid residue of the protein or polypeptide. Unless otherwise specified, sequences of polypeptides throughout the present disclosure are listed from N-terminus to C-terminus, and sequences of polynucleotides throughout the present disclosure are listed from the 5′ end to the 3′ end.

An “amino acid” as used herein refers to a compound including both a carboxyl (—COOH) and amino (—NH₂) group. “Amino acid” refers to both natural and unnatural, i.e., synthetic, amino acids. Natural amino acids, with their three-letter and single-letter abbreviations, include: alanine (Ala; A); arginine (Arg, R); asparagine (Asn; N); aspartic acid (Asp; D); cysteine (Cys; C); glutamine (Gln; Q); glutamic acid (Glu; E); glycine (Gly; G); histidine (His; H); isoleucine (Ile; I); leucine (Leu; L); lysine (Lys; K); methionine (Met; M); phenylalanine (Phe; F); proline (Pro; P); serine (Ser; S); threonine (Thr; T); tryptophan (Trp; W); tyrosine (Tyr; Y); and valine (Val; V). Unnatural or synthetic amino acids include a side chain that is distinct from the natural amino acids provided above and may include, e.g., fluorophores, post-translational modifications, metal ion chelators, photocaged and photo-cross-linked moieties, uniquely reactive functional groups, and NMR, IR, and x-ray crystallographic probes. Exemplary unnatural or synthetic amino acids are provided in, e.g., Mitra et al. (2013), Mater Methods 3:204 and Wals et al. (2014), Front Chem 2:15. Unnatural amino acids may also include naturally-occurring compounds that are not typically incorporated into a protein or polypeptide, such as, e.g., citrulline (Cit), selenocysteine (Sec), and pyrrolysine (Pyl).

As used herein, the terms “non-natural,” “non-naturally occurring,” “variant,” and “mutant” are used interchangeably in the context of an organism, polypeptide, or nucleic acid. The terms “non-natural,” “non-naturally occurring,” “variant,” and “mutant” in this context refer to a polypeptide or nucleic acid sequence having at least one variation or mutation at an amino acid position or nucleic acid position as compared to a wild-type polypeptide or nucleic acid sequence. The at least one variation can be, e.g., an insertion of one or more amino acids or nucleotides, a deletion of one or more amino acids or nucleotides, or a substitution of one or more amino acids or nucleotides. A “variant” protein or polypeptide is also referred to as a “non-natural” protein or polypeptide.

Naturally-occurring organisms, nucleic acids, and polypeptides can be referred to as “wild-type,” “wild type” or “original” or “natural” such as wild type strains of the referenced species, or a wild-type protein or nucleic acid sequence. Likewise, amino acids found in polypeptides of the wild type organism can be referred to as “original” or “natural” with regards to any amino acid position.

An “amino acid substitution” refers to a polypeptide or protein including one or more substitutions of wild-type or naturally occurring amino acid with a different amino acid relative to the wild-type or naturally occurring amino acid at that amino acid residue. The substituted amino acid may be a synthetic or naturally occurring amino acid. In some embodiments, the substituted amino acid is a naturally occurring amino acid selected from the group consisting of A, R, N, D, C, Q, E, G, H, I, L, K, M, F, P, S, T, W, Y, and V. In some embodiments, the substituted amino acid is an unnaturally or synthetic amino acid. Substitution mutants may be described using an abbreviated system. For example, a substitution mutation in which the fifth (5^{th}) amino acid residue is substituted may be abbreviated as “X5Y,” wherein “X” is the wild-type or naturally occurring amino acid to be replaced, “5” is the amino acid residue position within the amino acid sequence of the protein or polypeptide, and “Y” is the substituted, or non-wild-type or non-naturally occurring, amino acid.

An “isolated” polypeptide, protein, peptide, or nucleic acid is a molecule that has been removed from its natural environment. It is also understood that “isolated” polypeptides, proteins, peptides, or nucleic acids may be formulated with excipients such as diluents or adjuvants and still be considered isolated. As used herein, “isolated” does not necessarily imply any particular level purity of the polypeptide, protein, peptide, or nucleic acid.

The term “recombinant” when used in reference to a nucleic acid molecule, peptide, polypeptide, or protein means of, or resulting from, a new combination of genetic material that is not known to exist in nature. A recombinant molecule can be produced by any of the techniques available in the field of recombinant technology, including, but not limited to, polymerase chain reaction (PCR), gene splicing (e.g., using restriction endonucleases), and solid-phase synthesis of nucleic acid molecules, peptides, or proteins.

The term “domain” when used in reference to a polypeptide or protein means a distinct functional and/or structural unit in a protein. Domains are sometimes responsible for a particular function or interaction, contributing to the overall role of a protein. Domains may exist in a variety of biological contexts. Similar domains may be found in proteins with different functions. Alternatively, domains with low sequence identity (i.e., less than about 50%, less than about 40%, less than about 30%, less than about 20%, less than about 10%, less than about 5%, or less than about 1% sequence identity) may have the same function.

As used herein, the term “sequence similarity” (% similarity) refers to the degree of identity or correspondence between nucleic acid sequences or amino acid sequences. In the context of polynucleotides, “sequence similarity” may refer to nucleic acid sequences wherein changes in one or more nucleotide bases results in substitution of one or more amino acids, but do not affect the functional properties of the protein encoded by the polynucleotide. “Sequence similarity” may also refer to modifications of the polynucleotide, such as deletion or insertion of one or more nucleotide bases, that do not substantially affect the functional properties of the resulting transcript. It is therefore understood that the present disclosure encompasses more than the specific exemplary sequences. Methods of making nucleotide base substitutions are known, as are methods of determining the retention of biological activity of the encoded polypeptide.

In the context of polypeptides, “sequence similarity” refers to two or more polypeptides wherein greater than about 40% of the amino acids are identical, or greater than about 60% of the amino acids are functionally identical. “Functionally identical” or “functionally similar” amino acids have chemically similar side chains. For example, amino acids can be grouped in the following manner according to functional similarity: Positively-charged side chains: Arg, His, Lys; Negatively-charged side chains: Asp, Glu; Polar, uncharged side chains: Ser, Thr, Asn, Gln; Hydrophobic side chains: Ala, Val, Ile, Leu, Met, Phe, Tyr, Trp; Other: Cys, Gly, Pro.

In some embodiments, similar polypeptides of the present disclosure have about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% functionally identical amino acids.

The “percent identity” (% identity) between two polynucleotide or polypeptide sequences is determined when sequences are aligned for maximum homology, and generally not including gaps or truncations. Additional sequences added to a polypeptide sequence, such as but not limited to immunodetection tags, purification tags, localization sequences (presence or absence), etc., do not affect the % identity.

Algorithms known to those skilled in the art, such as Align, BLAST, ClustalW and others compare and determine a raw sequence similarity or identity, and also determine the presence or significance of gaps in the sequence which can be assigned a weight or score. Such algorithms also are known in the art and are similarly applicable for determining nucleotide or amino acid sequence similarity or identity, and can be useful in identifying orthologs of genes of interest.

In some embodiments, similar polynucleotides of the present disclosure have about 40%, at least about 40%, about 45%, at least about 45%, about 50%, at least about 50%, about 55%, at least about 55%, about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% identical nucleic acid sequence. In some embodiments, similar polypeptides of the present disclosure have about 40%, at least about 40%, about 45%, at least about 45%, about 50%, at least about 50%, about 55%, at least about 55%, about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% identical amino acid sequence.

A homolog is a gene or genes that are related by vertical descent and are responsible for substantially the same or identical functions in different organisms. Genes are related by vertical descent when, for example, they share sequence similarity of sufficient amount to indicate they are homologous or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Paralogs are genes related by duplication within a genome, and can evolve new functions, even if these are related to the original one.

An amino acid position (or simply, amino acid) “corresponding to” an amino acid position in another polypeptide sequence is the position that is aligned with the referenced amino acid position when the polypeptides are aligned for maximum homology, for example, as determined by BLAST, which allows for gaps in sequence homology within protein sequences to align related sequences and domains. Alternatively, in some instances, when polypeptide sequences are aligned for maximum homology, a corresponding amino acid may be the nearest amino acid to the identified amino acid that is within the same amino acid biochemical grouping—i.e., the nearest acidic amino acid, the nearest basic amino acid, the nearest aromatic amino acid, etc. to the identified amino acid.

By “substantially identical,” with reference to a nucleic acid sequence (e.g., a gene, RNA, or cDNA) or amino acid sequence (e.g., a protein or polypeptide) is meant one that has at least at least 80%, at least 85%, at least 90%, at least 95%, at least 96%, at least 97% at least 98%, or at least 99% nucleotide or amino acid identity, respectively, to a reference sequence.

As used in the context of proteins, the term “structural similarity” indicates the degree of homology between the overall shape, fold, and/or topology of the proteins. It should be understood that two proteins do not necessarily need to have high sequence similarity to achieve structural similarity. Protein structural similarity is often measured by root mean squared deviation (RMSD), global distance test score (GDT-score), and template modeling score (TM-score); see, e.g., Xu and Zhang (2010), Bioinformatics 26(7):889-895. Structural similarity can be determined, e.g., by superimposing protein structures obtained from, e.g., x-ray crystallography, NMR spectroscopy, cryogenic electron microscopy (cryo-EM), mass spectrometry, or any combination thereof, and calculating the RMSD, GDT-score, and/or TM-score based on the superimposed structures. In some embodiments, two proteins have substantially similar tertiary structures when the TM-score is greater than about 0.5, greater than about 0.6, greater than about 0.7, greater than about 0.8, or greater than about 0.9. In some embodiments, two proteins have substantially identical tertiary structures when the TM-score is about 1.0. Structurally-similar proteins may also be identified computationally using algorithms such as, e.g., TM-align (Zhang and Skolnick, Nucleic Acids Res 33(7):2302-2309, 2005); DALI (Holm and Sander, J Mol Biol 233(1):123-138, 1993); STRUCTAL (Gerstein and Levitt, Proc Int Conf Intell Syst Mol Biol 4:59-69, 1996); MINRMS (Jewett et al., Bioinformatics 19(5):625-634, 2003); Combinatorial Extension (CE) (Shindyalov and Bourne, Protein Eng 11(9):739-747, 1998); ProtDex (Aung et al., DASFAA 2003, Proceedings); VAST (Gibrat et al., Curr Opin Struct Biol 6:377-385, 1996); LOCK (Singh and Brutlag, Proc Int Conf Intell Syst Mol Biol 5:284-293, 1997); SSM (Krissinel and Henrick, Acta Cryst D60:2256-2268, 2004), and the like.

### Flavin-Dependent Oxidase

Cannabinoid synthases are enzymes responsible for the biosynthesis of cannabinoids, e.g., cannabinoid compounds described herein. The only naturally-occurring cannabinoid synthase enzymes currently known to convert cannabigerolic acid (CBGA) or its analogs to cannabinoids such as Δ9-tetrahydrocannabinolic acid (THCA) by THCA synthase (THCAS, EC 1.21.3.7), cannabidiolic acid (CBDA) by CBDA synthase (CBDAS, EC 1.21.3.8) or cannabichromenic acid (CBCA) by CBCA synthase (CBCAS) or their analogs are from the plant Cannabis sativa (Onofri et al. (2015), J Mol Biol 423:96; Laverty et al. (2019), Genome Research 29:146-156). It is challenging to utilize these enzymes from C. sativa for heterologous cannabinoid production in microorganisms such as bacteria because they are typically secreted proteins that require a disulfide bond and glycosylation, are poorly active, and require low pH for optimal activity (Zirpel et al. (2018), J Biotechnol 284:17-26). Thus, cannabinoid synthase enzymes from C. sativa are not conducive for standard microbial fermentation processes that typically use media with a neutral or near neutral pH of 6 to 8.

The present inventors have discovered and engineered alternative enzymes for the improved microbial production of cannabinoids. The enzymes described herein do not contain a disulfide bond, do not require glycosylation, and are active at neutral pH. Thus, these enzymes are suitable for soluble and active expression in a microbial host under standard fermentation conditions. In some embodiments, the enzyme is a bacterial or fungal enzyme.

In some embodiments, the disclosure provides a non-natural flavin-dependent oxidase comprising at least one amino acid variation as compared to a wild type flavin-dependent oxidase, wherein the non-natural flavin-dependent oxidase does not comprise a disulfide bond, and wherein the non-natural flavin-dependent oxidase is capable of oxidative cyclization of a prenylated aromatic compound into a cannabinoid.

As used herein, “cannabinoid” refers to a prenylated polyketide or terpenophenolic compound derived from fatty acid or isoprenoid precursors. In general, cannabinoids are produced via a multi-step biosynthesis pathway, with the final precursor being a prenylated aromatic compound. In some embodiments, the prenylated aromatic compound is cannabigerolic acid (CBGA), cannabigerorcinic acid (CBGOA), cannabigerivarinic acid (CBGVA), cannabigerorcinol (CBGO), cannabigerivarinol (CBGV), or cannabigerol (CBG). In some embodiments, the prenylated aromatic compound is converted into a cannabinoid by oxidative cyclization. In some embodiments, the non-natural flavin-dependent oxidase converts one or more of CBGA, CBGOA, CBGVA, CBGO, CBGV, and CBG into a cannabinoid.

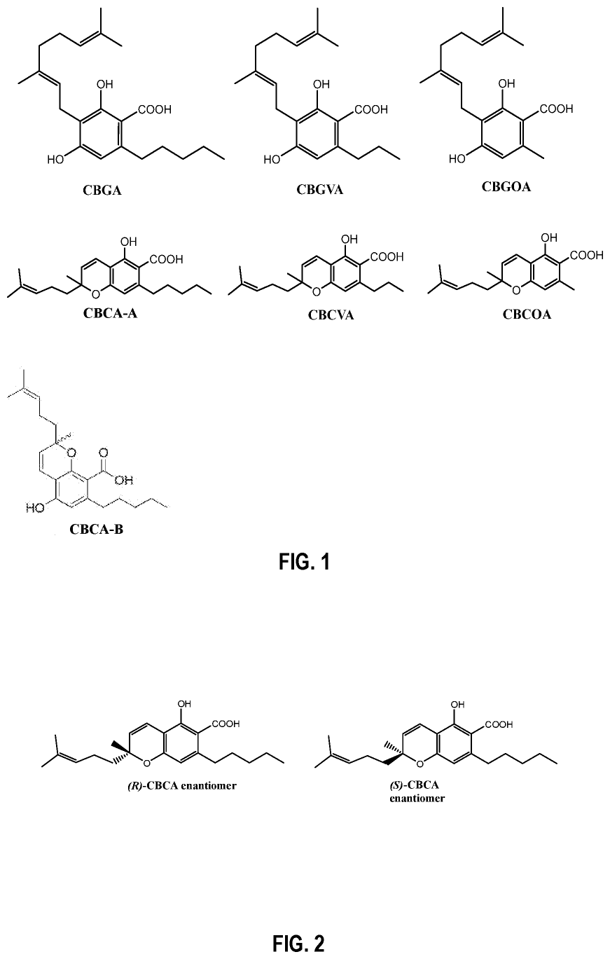

In some embodiments, the non-natural flavin-dependent oxidase converts CBGA into one or more of CBCA, CBDA, or THCA. In some embodiments, the non-natural flavin-dependent oxidase converts CBGA into CBCA. In some embodiments, “CBCA” as used throughout the present disclosure refers to the CBCA-A regioisomer of CBCA, as shown in FIG. 1. It will be understood by one of ordinary skill in the art that a non-natural flavin-dependent oxidase described herein that is capable of producing CBCA may be capable of producing one or both regioisomers of CBCA, i.e., CBCA-A and CBCA-B. In some embodiments, the non-natural flavin-dependent oxidase converts CBGOA into one or more of CBCOA, CBDOA, or THCOA. In some embodiments, the non-natural flavin-dependent oxidase converts CBGOA into CBCOA. In some embodiments, the non-natural flavin-dependent oxidase converts CBGVA into one or more of CBCVA, CBDVA, or THCVA. In some embodiments, the non-natural flavin-dependent oxidase converts CBGVA into CBCVA. In some embodiments, the non-natural flavin-dependent oxidase converts CBGO into one or more of CBCO, CBDO, or THCO. In some embodiments, the non-natural flavin-dependent oxidase converts CBGO into CBCO. In some embodiments, the non-natural flavin-dependent oxidase converts CBGV into one or more of CBCV, CBDV, or THCV.

In some embodiments, the non-natural flavin-dependent oxidase converts CBGV into CBCV. In some embodiments, the non-natural flavin-dependent oxidase converts CBG into one or more of CBC, CBD, or THC. In some embodiments, the non-natural flavin-dependent oxidase converts CBG into one or more of CBC.

Different cannabinoids can be produced based on the way that a precursor is cyclized. For example, THCA, CBDA, and CBCA are produced by oxidative cyclization of CBGA. Further examples of cannabinoids include, but are not limited to, THCA, THCV, THCO, THCVA, THCOA, THC, CBDA, CBDV, CBDO, CBDVA, CBDOA, CBD, CBCA, CBCV, CBCO, CBCVA, CBCOA, CBC, cannabinolic acid (CBNA), cannabinol (CBN), cannabicyclol (CBL), cannabivarin (CBV), cannabielsoin (CBE), cannabicitran, and isomers, analogs or derivatives thereof. As used herein, an “isomer” of a reference compound has the same molecular formula as the reference compound, but with a different arrangement of the atoms in the molecule. For example, CBCA-A and CBCA-B, as shown in FIG. 1, are regioisomers of CBCA. As used herein, an “analog” or “structural analog” of a reference compound has a similar structure as the reference compound, but differs in a certain component such as an atom, a functional group, or a substructure. An analog can be imagined to be formed from the reference compound, but not necessarily formed or derived from the reference compound. As used herein, a “derivative” of a reference compound is derived from a similar compound by a similar reaction. Methods of identifying isomers, analogs or derivatives of the cannabinoids described herein are known to one of ordinary skill in the art.

In some embodiments, the non-natural flavin-dependent oxidase is a berberine bridge enzyme (BBE)-like enzyme. BBE-like enzymes are described, e.g., in Daniel et al. (2017), Arch Biochem Biophys 632:88-103 and include protein family domains (Pfams) PF08031 (berberine-bridge domain) and PF01564 (flavin adenine dinucleotide (FAD)-binding domain). In general, a BBE-like enzyme comprises a FAD binding module that is formed by the N- and C-terminal portions of the protein, and a central substrate binding domain that, together with the FAD cofactor, provides the environment for efficient substrate binding, oxidation and cyclization. A non-limiting list of BBE-like enzymes and are described in Table 1. It will be understood by one of ordinary skill in the art that, in some embodiments, a BBE-like enzyme binds a flavin mononucleotide (FMN) in addition to or instead of FAD.

TABLE 1
Exemplary BBE-like enzymes with published structures.

| Protein | Organism | PDB Entries | Cofactor(s), ligand(s) |
|---|---|---|---|
| Reticuline Oxidase/ | Eschscholzia | 3D2D, 3D2H, 3D2J, | FAD, (S)-reticuline, (S)- |
| Dehydrogenase | californica | 3FW7, 3WF8, 3WF9, | scoulerine, dehydroscoulerine |
| | | 3WFA, 3GSY, 4EC3, | |
| | | 4PZF | |
| Pollen Allergen | Phleum pratense | 3TSH, 3TSJ, 4PVE, | FAD |
| PhI p 4.0202 | | 4PVH, 4PVK, 4PVJ, | |
| | | 4PWB, 4PWC, | |
| MaDA | Morus alba | 6JQH | FAD |
| THCA Synthase | Cannabis sativa | 3VTE | FAD |
| Pyrimidine | Escherichia coli | 6SGG, 6SGL, 6SGM, | FMN, 2,4- |
| monooxygenase | K-12 | 6SGN, 6TEE, 6TEF, | dimethoxypyrimidine |
| RutA | | 6TEG | |
| Caerulomycin | Actinoalloteichus | 5I1V, 5I1W | FAD, 6-(hydroxymethyl)[2,2′- |
| Oxidase K (CrmK) | sp. WH1-2216-6 | | bipyridin]-4-ol, 4-hydroxy[2,2′- |
| | | | bipyridine]-6-carbaldehyde |
| Tirandamycin | Streptomyces | 2Y08, 2Y3R, 2Y3S, | FAD, Tirandamycin D, |
| Oxidase L (TamL) | sp. 307-9 | 2Y4G | Tirandamycin E |
| EncM | Streptomyces | 3W8W, 3W8X, 3W8Z, | FAD, 6,6,6-trifluoro-1- |
| | maritimus | 4XLO, 6FOQ, 6FOW, | phenylhexane-1,3,5-trione, |
| | | 6FP3, 6FY8, 6FY9, | (7S)-7-hydroxy-1- |
| | | 6FYA, 6FYB, 6FYC, | phenyloctane-1,3,5-trione |
| | | 6FYD, 6FYE, 6FYF, | |
| | | 6FYG | |

In some embodiments, the non-natural flavin-dependent oxidase has substantial structural similarity with a cannabinoid synthase from C. sativa, e.g., Δ9-tetrahydrocannabinolic acid synthase (THCAS). THCAS utilizes a FAD cofactor when catalyzing the conversion of substrate CBGA to THCA. In some embodiments, the enzyme comprises a structurally similar active site as a cannabinoid synthase from C. sativa, e.g., THCAS. As used herein, the term “active site” refers to one or more regions in an enzyme that may be important for catalysis, substrate binding, and/or cofactor binding.

In some embodiments, the non-natural flavin-dependent oxidase has at least 3000 sequence identity to any one of SEQ ID NOs:1-3, any one of SEQ ID NOs:10-13, or any one of SEQ ID NOs:17-19. In some embodiments, the non-natural flavin-dependent oxidase has at least 40% sequence identity to any one of SEQ ID NOs:1-3, any one of SEQ ID NOs:10-13, or any one of SEQ ID NOs:17-19. In some embodiments, the non-natural flavin-dependent oxidase has at least 70% sequence identity to any one of SEQ ID NOs:1-3, any one of SEQ ID NOs:10-13, or any one of SEQ ID NOs:17-19. In some embodiments, the non-natural flavin-dependent oxidase has at least 50% sequence identity to any one of SEQ ID NOs:1-3, any one of SEQ ID NOs:10-13, or any one of SEQ ID NOs:17-19. In some embodiments, the non-natural flavin-dependent oxidase has at least 60% sequence identity to any one of SEQ ID NOs:1-3, any one of SEQ ID NOs:10-13, or any one of SEQ ID NOs:17-19. In some embodiments, the non-natural flavin-dependent oxidase has at least 70% sequence identity to any one of SEQ ID NOs:1-3, any one of SEQ ID NOs:10-13, or any one of SEQ ID NOs:17-19. In some embodiments, the non-natural flavin-dependent oxidase has at least 80% sequence identity to any one of SEQ ID NOs:1-3, any one of SEQ ID NOs:10-13, or any one of SEQ ID NOs:17-19. In some embodiments, the non-natural flavin-dependent oxidase has at least 85% sequence identity to any one of SEQ ID NOs:1-3, any one of SEQ ID NOs:10-13, or any one of SEQ ID NOs:17-19. In some embodiments, the non-natural flavin-dependent oxidase has at least 90% sequence identity to any one of SEQ ID NOs:1-3, any one of SEQ ID NOs:10-13, or any one of SEQ ID NOs:17-19. In some embodiments, the non-natural flavin-dependent oxidase has at least 95% sequence identity to any one of SEQ ID NOs:1-3, any one of SEQ ID NOs:10-13, or any one of SEQ ID NOs:17-19. In some embodiments, the non-natural flavin-dependent oxidase comprises any one of SEQ ID NOs:1-3, any one of SEQ ID NOs:10-13, or any one of SEQ ID NOs:17-19.

As described herein, a “non-natural” protein or polypeptide refers to a protein or polypeptide sequence having at least one variation at an amino acid position as compared to a wild-type polypeptide sequence. In some embodiments, the non-natural flavin-dependent oxidase has at least one variation at an amino acid position as compared to a wild-type flavin-dependent oxidase.

In some embodiments, the at least one amino acid variation comprises a substitution, deletion, insertion, or a combination thereof. In some embodiments, the variation comprises an amino acid substitution. In some embodiments, the variation comprises a deletion of one or more amino acids e.g., about 1 to about 100, about 2 to about 80, about 5 to about 50, about 10 to about 40, about 12 to about 35, about 13 to about 32, or about 14 to about 30 amino acids. In some embodiments, the variation comprises an insertion of one or more amino acids. In some embodiments, the at least one amino acid variation in the non-natural flavin-dependent oxidase is not in an active site of the flavin-dependent oxidase. In some embodiments, the active site of the flavin-dependent oxidase comprises one or more amino acid residues involved in binding the substrate, e.g., CBGA, CBGOA, CBGVA, CBGO, CBGV, and/or CBG. In some embodiments, the active site of the flavin-dependent oxidase comprises one or more amino acid residues involved in binding FAD cofactor. In some embodiments, the active site of the flavin-dependent oxidase comprises one or more amino acid residues involved for catalysis, e.g., the oxidative cyclization of CBGA into CBCA.

In some embodiments, the non-natural flavin-dependent oxidase does not comprise a disulfide bond. In the context of a protein or polypeptide, a disulfide bond (sometimes called a “S—S bond” or “disulfide bridge”) refers to a covalent bond between two cysteine residues, typically formed through oxidation of the thiol groups on the cysteines. Proteins comprising disulfide bonds, e.g., endogenous to plants, can be unstable in bacterial host cells as the disulfide bonds are often disrupted due to the reducing environment in bacterial cells. In some embodiments, cannabinoid synthases from C. sativa are substantially unstable in a bacterial cell, e.g., an E. coli cell. As used herein, “unstable” protein can refer to proteins that are non-functional, denatured, and/or degraded rapidly, resulting in catalytic activity that is greatly reduced relative to the activity found in its native host cell, e.g., C. sativa plants. In some embodiments, the lack of a disulfide bond in the non-natural flavin-dependent oxidase advantageously allows for its soluble and active expression by a bacterial host cell. In some embodiments, a bacterial host cell produces at least 1.5 times, at least 1.6 times, at least 1.7 times, at least 1.8 times, at least 1.9 times, at least 2 times, at least 3 times, at least 4 times, at least 5 times, at least 6 times, at least 7 times, at least 8 times, at least 9 times, at least 10 times, at least 20 times, at least 50 times, or at least 100 times more of the non-natural flavin-dependent oxidase that does not comprise a disulfide bond as compared with a flavin-dependent oxidase that comprises a disulfide bond, e.g., a wild-type cannabinoid synthase from C. sativa.

In some embodiments, the non-natural flavin-dependent oxidase is not glycosylated. As used herein, glycosylation refers to the addition of one or more sugar molecules to another biomolecule, e.g., a protein or polypeptide. Glycosylation can play an important role in the folding, secretion, and stability of proteins (see, e.g., Drickamer and Taylor, Introduction to Glycobiology (2^{nd} ed.), Oxford University Press, USA). Glycosylation mechanisms and patterns in bacteria and eukaryotes are distinct from one another. Moreover, the most common type of glycosylation, N-linked glycosylation, occurs in eukaryotes but not in bacteria. Thus, bacterial cells are generally not suitable for the production of eukaryotic proteins that are glycosylated, e.g., the cannabinoid synthases from C. sativa. In some embodiments, the lack of glycosylation in the non-natural flavin-dependent oxidase further advantageously allows for its soluble and active expression by a bacterial host cell. In some embodiments, a bacterial host cell produces at least 1.5 times, at least 1.6 times, at least 1.7 times, at least 1.8 times, at least 1.9 times, at least 2 times, at least 3 times, at least 4 times, at least 5 times, at least 6 times, at least 7 times, at least 8 times, at least 9 times, at least 10 times, at least 20 times, at least 50 times, or at least 100 times more (e.g., by weight) of the non-natural flavin-dependent oxidase that is not glycosylated, compared with a flavin-dependent oxidase that is glycosylated, e.g., a wild-type cannabinoid synthase from C. sativa.

In some embodiments, a bacterial host cell produces at least 1.5 times, at least 1.6 times, at least 1.7 times, at least 1.8 times, at least 1.9 times, at least 2 times, at least 3 times, at least 4 times, at least 5 times, at least 6 times, at least 7 times, at least 8 times, at least 9 times, at least 10 times, at least 20 times, at least 50 times, or at least 100 times more of the non-natural flavin-dependent oxidase that does not comprise a disulfide bond and is not glycosylated, compared with a flavin-dependent oxidase that comprises a disulfide bond and is glycosylated, e.g., a wild-type cannabinoid synthase from C. sativa.

In some embodiments, the non-natural flavin-dependent oxidase utilizes a flavin cofactor, e.g., FAD or FMN, for catalytic activity. In some embodiments, the non-natural flavin-dependent oxidase utilizes a FAD cofactor for catalytic activity, e.g., the conversion of a prenylated aromatic compound such as CBGA, CBGOA, CBGVA, CBG, CBGO, and/or CBGV into a cannabinoid. In some embodiments, the non-natural dependent oxidase comprises a monovalently bound FAD cofactor. As used herein, “monovalently bound” means that the FAD is covalently bound to one amino acid residue of the referenced protein, e.g., the non-natural flavin-dependent oxidase. In some embodiments, the non-natural flavin-dependent oxidase comprises a bivalently bound FAD cofactor. As used herein, “bivalently bound” means that the FAD is covalently bound to two amino acid residues of the referenced protein, e.g., the non-natural flavin-dependent oxidase. In some embodiments, the cannabinoid synthases from C. sativa comprise bivalently bound FAD cofactor. In some embodiments, the FAD cofactor is covalently bound to a histidine and/or a cysteine of the non-natural flavin-dependent oxidase.

In some embodiments, catalytic activity of the non-natural flavin-dependent oxidase is substantially the same from about pH 4 to about pH 9. In some embodiments, catalytic activity of the non-natural flavin-dependent oxidase is substantially the same from about pH 4.5 to about pH 8.5. In some embodiments, catalytic activity of the non-natural flavin-dependent oxidase is substantially the same from about pH 5 to about pH 8. In some embodiments, catalytic activity of the non-natural flavin-dependent oxidase is substantially the same from about pH 5.5 to about pH 7.5. In some embodiments, catalytic activity of the non-natural flavin-dependent oxidase is substantially the same from about pH 5 to about pH 7. In some embodiments, catalytic activity of the non-natural flavin-dependent oxidase is substantially the same at about pH 5 and at about pH 7. The term “substantially” when referring to enzyme activity at different pH conditions means that the non-natural flavin-dependent oxidase enzyme activity does not vary (increase or decrease) by more than 20%, more than 15%, more than 10%, more than 5%, or more than 1% under the different pH conditions. In some embodiments, catalytic activity of the non-natural flavin-dependent oxidase does not vary more than 20%, more than 15%, more than 10%, more than 5%, or more than 1% from about pH 5 to about pH 8. As described herein, cannabinoid synthases from C. sativa generally require low pH (around 5 to 5.5) for optimal activity and are less active at neutral pH (see, e.g., Zirpel et al. (2018), J Biotechnol 284:17-26). The catalytic activity of the non-natural flavin-dependent oxidase does not vary substantially over a wide range of pH (e.g., from about pH 5 to about pH 8), which is beneficial for microbial production of cannabinoids.

In some embodiments, the non-natural flavin-dependent oxidase has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to a natural, i.e., wild-type, flavin-dependent oxidase. As described herein, the terms “natural” or “wild-type” flavin-dependent oxidase can refer to any known flavin-dependent oxidase sequence. For example, a natural flavin-dependent oxidase can include, but is not limited to, Tcz9 from Streptomyces sp. AJS327 (accession ID WP_180931280.1), a protein from Phytohabitans suffuscus with the accession ID WP_173153368.1 (“Psx9”), a protein from Streptomyces varsoviensis with the accession ID WP_030877494.1 (“Svx9”), a protein from Streptomyces olivochromogenes with the accession ID WP_235454663.1 (“Sox9”), ThdO from Streptomyces albogriseolus (accession ID ANW12117.1), ThnG from Streptomyces sp. FXJ1.172 (accession ID WP_067055472.1), a protein from Streptomyces sp. Ru87 with the accession ID WP_098755161.1 (“SrThX”), a protein from Streptomyces lycii with the accession ID WP_156205671.1 (“Slx9”), a protein from Frankia sp. CiP3 with the accession ID WP_239335163.1 (“Frx9”), a protein from Kutzneria sp. CA-103260 with the accession ID WP_211768552.1 (“Kcx9”), and the proteins listed in Table 1.

In some embodiments, the disclosure provides a non-natural flavin-dependent oxidase with about 70%, 75%, 80%, 85%, 90%, 95%, 99% or greater identity to at least about 25, 50, 75, 100, 125, 150, 200, 250, 300, 350, 400, 450, 500, or more contiguous amino acids of any one of SEQ ID NOs:1-3, any one of SEQ ID NOs:10-13, or any one of SEQ ID NOs:17-19, comprising at least one amino acid variation as compared to a wild type flavin-dependent oxidase, wherein the non-natural flavin-dependent oxidase converts one or more of cannabigerolic acid (CBGA), cannabigerorcinic acid (CBGOA), cannabigerivarinic acid (CBGVA), cannabigerol (CBG), cannabigerorcinol (CBGO), and cannabigerivarinol (CBGV) into a cannabinoid. In some embodiments, the non-natural flavin-dependent oxidase converts CBGA into CBCA. In some embodiments, the non-natural flavin-dependent oxidase converts CBGA into CBDA. In some embodiments, the non-natural flavin-dependent oxidase converts CBGA into THCA. In some embodiments, the non-natural flavin-dependent oxidase comprises about 70%, 75%, 80%, 85%, 90%, 95%, 99% or greater identity to at least about 25, 50, 75, 100, 125, 150, 200, 250, 300, 350, 400, 450, 500, or more contiguous amino acids of any one of SEQ ID NOs:1-3, SEQ ID NO:10, SEQ ID NO:11, SEQ ID NO:13, or SEQ ID NO:19.

In some embodiments, the non-natural flavin-dependent oxidase has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:1. SEQ ID NO:1 describes the amino acid sequence of the Tcz9 protein from Streptomyces sp. AJS327, which has the accession ID WP_180931280.1 and is referred to herein as “Tcz9.”

In some embodiments, the non-natural flavin-dependent oxidase has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:2. SEQ ID NO:2 describes the amino acid sequence of a protein from Phytohabitans suffuscus with the accession ID WP_173153368.1, referred to herein as “Psx9.”

In some embodiments, the non-natural flavin-dependent oxidase has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:3. SEQ ID NO:3 describes the amino acid sequence of a protein from Streptomyces varsoviensis with the accession ID WP_030877494.1, referred to herein as “Svx9.”

In some embodiments, the non-natural flavin-dependent oxidase has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:10. SEQ ID NO:10 describes the amino acid sequence of a protein from Streptomyces olivochromogenes with the accession ID WP_235454663.1, referred to herein as “Sox9.”

In some embodiments, the non-natural flavin-dependent oxidase has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:11. SEQ ID NO:11 describes the amino acid sequence of the ThdO protein from Streptomyces albogriseolus, which has the accession ID ANW12117.1 and is referred to herein as “ThdO.”

In some embodiments, the non-natural flavin-dependent oxidase has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:12. SEQ ID NO:12 describes the amino acid sequence of the ThnG protein from Streptomyces sp. FXJ1.172, which has the accession ID WP_067055472.1 and is referred to herein as “ThnG.”

In some embodiments, the non-natural flavin-dependent oxidase has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:13. SEQ ID NO:13 describes the amino acid sequence of a protein from Streptomyces sp. Ru87 with the accession ID WP_098755161.1, referred to herein as “SrThX.”

In some embodiments, the non-natural flavin-dependent oxidase has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:17. SEQ ID NO:17 describes the amino acid sequence of a protein from Streptomyces lycii with the accession ID WP_156205671.1, referred to herein as “Slx9.”

In some embodiments, the non-natural flavin-dependent oxidase has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:18. SEQ ID NO:18 describes the amino acid sequence of a protein from Frankia sp. CiP3 with the accession ID WP_239335163.1, referred to herein as “Frx9.”

In some embodiments, the non-natural flavin-dependent oxidase has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:19. SEQ ID NO:19 describes the amino acid sequence of a protein from Kutzneria sp. CA-103260 with the accession ID WP_211768552.1, referred to herein as “Kcx9.”

The present inventors found that the Tcz9 enzyme from Streptomyces sp. AJS327, the Psx9 enzyme from Phytohabitans suffuscus, the Svx9 enzyme from Streptomyces varsoviensis, the Sox9 enzyme from Streptomyces olivochromogenes, the ThdO enzyme from Streptomyces albogriseolus, the ThnG enzyme from Streptomyces sp. FXJ1.172, the SrThX enzyme from Streptomyces sp. Ru87, the Slx9 enzyme from Streptomyces lycii, the Frx9 enzyme from Frankia sp. CiP3, and the Kcx9 enzyme from Kutzneria sp. CA-103260 may be homologs of the BBE-like enzyme Clz9 from Streptomyces sp. CNH-287, which may have a similar catalytic mechanism as a cannabinoid synthase, e.g., THCAS, CBDAS, or CBCAS, from C. sativa. Clz9 is involved in biosynthesis of the tetrachlorinated alkaloid Chlorizidine A (see, e.g., Mantovani et al., J Am Chem Soc 135:18032-18035 (2013)), while Tcz9 is involved in Tetrachlorizine biosynthesis in Streptomyces sp. AJS327 (see, e.g., Purdy et al. 2021, J Am Chem Soc 143(10):3682-3686 (2021)). Psx9, Svx9, and Sox9 were found to be part of unknown biosynthesis gene clusters in Phytohabitans suffuscus, Streptomyces varsoviensis, and Streptomyces olivochromogenes, respectively, that resembled the Chlorizidine and Tetrachlorizine gene clusters found in Streptomyces sp. CNH-287 and/or Streptomyces sp. AJS327. Further, ThdO and ThnG were found to be associated with the thienodolin biosynthesis gene cluster in Streptomyces albogriseolus and Streptomyces sp. FXJ1.172, respectively, and SrThX was found to be associated with an unknown thienodolin-like biosynthesis gene cluster in Streptomyces sp. Ru87. Slx9, Frx9, and Kcx9 were also identified as having potential sequences of interest. Thus, Tcz9, Psx9, Svx9, Sox9, ThdO, ThnG, SrThX, Slx9, Frx9, and Kcx9 may share a similar catalytic mechanism as cannabinoid synthases such as THCAS, CBDAS, and CBCAS from C. sativa. The present inventors further noticed that Tcz9, Psx9, Svx9, Sox9, ThdO, SrThX, and Kcx9 accept the cannabinoid precursor CBGA as substrate.

In some embodiments, the non-natural flavin-dependent oxidase comprising at least 70%, at least 80%, least at least 85%, at least 90% or at least 95% sequence identity to SEQ ID NO:1 comprises 1 to 100, 1 to 90, 1 to 80, 1 to 70, 1 to 60, 1 to 50, 1 to 40, 1 to 30, 1 to 25, 1 to 20, 2 to 20, 3 to 20, 4 to 20, 5 to 20, 6 to 20, 7 to 20, 8 to 20, 9 to 20, 10 to 20, 11 to 20, 12 to 20, 13 to 20, 14 to 20, 15 to 20, 16 to 20, 17 to 20, 18 to 20, or 19 to 20 amino acid variations as compared to wild-type Tcz9. In some embodiments, the non-natural flavin-dependent oxidase comprises about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, or about 100 amino acid variations as compared to wild-type Tcz9. In some embodiments, the amino acid variation in the non-natural flavin-dependent oxidase is an amino acid substitution, deletion, or insertion. In some embodiments, the variation is a substitution of one or more amino acids in the wild-type Tcz9 polypeptide sequence. In some embodiments, the variation is a deletion of one or more amino acids in the wild-type Tcz9 polypeptide sequence. In some embodiments, the variation is an insertion of one or more amino acids in the wild-type Tcz9 polypeptide sequence.

In some embodiments, the non-natural flavin-dependent oxidase comprising at least 70%, at least 80%, least at least 85%, at least 90% or at least 95% sequence identity to SEQ ID NO:2 comprises 1 to 100, 1 to 90, 1 to 80, 1 to 70, 1 to 60, 1 to 50, 1 to 40, 1 to 30, 1 to 25, 1 to 20, 2 to 20, 3 to 20, 4 to 20, 5 to 20, 6 to 20, 7 to 20, 8 to 20, 9 to 20, 10 to 20, 11 to 20, 12 to 20, 13 to 20, 14 to 20, 15 to 20, 16 to 20, 17 to 20, 18 to 20, or 19 to 20 amino acid variations as compared to wild-type Psx9. In some embodiments, the non-natural flavin-dependent oxidase comprises about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, or about 100 amino acid variations as compared to wild-type Psx9. In some embodiments, the amino acid variation in the non-natural flavin-dependent oxidase is an amino acid substitution, deletion, or insertion. In some embodiments, the variation is a substitution of one or more amino acids in the wild-type Psx9 polypeptide sequence. In some embodiments, the variation is a deletion of one or more amino acids in the wild-type Psx9 polypeptide sequence. In some embodiments, the variation is an insertion of one or more amino acids in the wild-type Psx9 polypeptide sequence.

In some embodiments, the non-natural flavin-dependent oxidase comprising at least 70%, at least 80%, least at least 85%, at least 90% or at least 95% sequence identity to SEQ ID NO:3 comprises 1 to 100, 1 to 90, 1 to 80, 1 to 70, 1 to 60, 1 to 50, 1 to 40, 1 to 30, 1 to 25, 1 to 20, 2 to 20, 3 to 20, 4 to 20, 5 to 20, 6 to 20, 7 to 20, 8 to 20, 9 to 20, 10 to 20, 11 to 20, 12 to 20, 13 to 20, 14 to 20, 15 to 20, 16 to 20, 17 to 20, 18 to 20, or 19 to 20 amino acid variations as compared to wild-type Svx9. In some embodiments, the non-natural flavin-dependent oxidase comprises about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, or about 100 amino acid variations as compared to wild-type Svx9. In some embodiments, the amino acid variation in the non-natural flavin-dependent oxidase is an amino acid substitution, deletion, or insertion. In some embodiments, the variation is a substitution of one or more amino acids in the wild-type Svx9 polypeptide sequence. In some embodiments, the variation is a deletion of one or more amino acids in the wild-type Svx9 polypeptide sequence. In some embodiments, the variation is an insertion of one or more amino acids in the wild-type Svx9 polypeptide sequence.

In some embodiments, the non-natural flavin-dependent oxidase comprising at least 70%, at least 80%, least at least 85%, at least 90% or at least 95% sequence identity to SEQ ID NO:10 comprises 1 to 100, 1 to 90, 1 to 80, 1 to 70, 1 to 60, 1 to 50, 1 to 40, 1 to 30, 1 to 25, 1 to 20, 2 to 20, 3 to 20, 4 to 20, 5 to 20, 6 to 20, 7 to 20, 8 to 20, 9 to 20, 10 to 20, 11 to 20, 12 to 20, 13 to 20, 14 to 20, 15 to 20, 16 to 20, 17 to 20, 18 to 20, or 19 to 20 amino acid variations as compared to wild-type Sox9. In some embodiments, the non-natural flavin-dependent oxidase comprises about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, or about 100 amino acid variations as compared to wild-type Sox9. In some embodiments, the amino acid variation in the non-natural flavin-dependent oxidase is an amino acid substitution, deletion, or insertion. In some embodiments, the variation is a substitution of one or more amino acids in the wild-type Sox9 polypeptide sequence. In some embodiments, the variation is a deletion of one or more amino acids in the wild-type Sox9 polypeptide sequence. In some embodiments, the variation is an insertion of one or more amino acids in the wild-type Sox9 polypeptide sequence.

In some embodiments, the non-natural flavin-dependent oxidase comprising at least 70%, at least 80%, least at least 85%, at least 90% or at least 95% sequence identity to SEQ ID NO:11 comprises 1 to 100, 1 to 90, 1 to 80, 1 to 70, 1 to 60, 1 to 50, 1 to 40, 1 to 30, 1 to 25, 1 to 20, 2 to 20, 3 to 20, 4 to 20, 5 to 20, 6 to 20, 7 to 20, 8 to 20, 9 to 20, 10 to 20, 11 to 20, 12 to 20, 13 to 20, 14 to 20, 15 to 20, 16 to 20, 17 to 20, 18 to 20, or 19 to 20 amino acid variations as compared to wild-type ThdO. In some embodiments, the non-natural flavin-dependent oxidase comprises about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, or about 100 amino acid variations as compared to wild-type ThdO. In some embodiments, the amino acid variation in the non-natural flavin-dependent oxidase is an amino acid substitution, deletion, or insertion. In some embodiments, the variation is a substitution of one or more amino acids in the wild-type ThdO polypeptide sequence. In some embodiments, the variation is a deletion of one or more amino acids in the wild-type ThdO polypeptide sequence. In some embodiments, the variation is an insertion of one or more amino acids in the wild-type ThdO polypeptide sequence.

In some embodiments, the non-natural flavin-dependent oxidase comprising at least 70%, at least 80%, least at least 85%, at least 90% or at least 95% sequence identity to SEQ ID NO:12 comprises 1 to 100, 1 to 90, 1 to 80, 1 to 70, 1 to 60, 1 to 50, 1 to 40, 1 to 30, 1 to 25, 1 to 20, 2 to 20, 3 to 20, 4 to 20, 5 to 20, 6 to 20, 7 to 20, 8 to 20, 9 to 20, 10 to 20, 11 to 20, 12 to 20, 13 to 20, 14 to 20, 15 to 20, 16 to 20, 17 to 20, 18 to 20, or 19 to 20 amino acid variations as compared to wild-type ThnG. In some embodiments, the non-natural flavin-dependent oxidase comprises about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, or about 100 amino acid variations as compared to wild-type ThnG. In some embodiments, the amino acid variation in the non-natural flavin-dependent oxidase is an amino acid substitution, deletion, or insertion. In some embodiments, the variation is a substitution of one or more amino acids in the wild-type ThnG polypeptide sequence. In some embodiments, the variation is a deletion of one or more amino acids in the wild-type ThnG polypeptide sequence. In some embodiments, the variation is an insertion of one or more amino acids in the wild-type ThnG polypeptide sequence.

In some embodiments, the non-natural flavin-dependent oxidase comprising at least 70%, at least 80%, least at least 85%, at least 90% or at least 95% sequence identity to SEQ ID NO:13 comprises 1 to 100, 1 to 90, 1 to 80, 1 to 70, 1 to 60, 1 to 50, 1 to 40, 1 to 30, 1 to 25, 1 to 20, 2 to 20, 3 to 20, 4 to 20, 5 to 20, 6 to 20, 7 to 20, 8 to 20, 9 to 20, 10 to 20, 11 to 20, 12 to 20, 13 to 20, 14 to 20, 15 to 20, 16 to 20, 17 to 20, 18 to 20, or 19 to 20 amino acid variations as compared to wild-type SrThX. In some embodiments, the non-natural flavin-dependent oxidase comprises about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, or about 100 amino acid variations as compared to wild-type SrThX. In some embodiments, the amino acid variation in the non-natural flavin-dependent oxidase is an amino acid substitution, deletion, or insertion. In some embodiments, the variation is a substitution of one or more amino acids in the wild-type SrThX polypeptide sequence. In some embodiments, the variation is a deletion of one or more amino acids in the wild-type SrThX polypeptide sequence. In some embodiments, the variation is an insertion of one or more amino acids in the wild-type SrThX polypeptide sequence.

In some embodiments, the non-natural flavin-dependent oxidase comprising at least 70%, at least 80%, least at least 85%, at least 90% or at least 95% sequence identity to SEQ ID NO:17 comprises 1 to 100, 1 to 90, 1 to 80, 1 to 70, 1 to 60, 1 to 50, 1 to 40, 1 to 30, 1 to 25, 1 to 20, 2 to 20, 3 to 20, 4 to 20, 5 to 20, 6 to 20, 7 to 20, 8 to 20, 9 to 20, 10 to 20, 11 to 20, 12 to 20, 13 to 20, 14 to 20, 15 to 20, 16 to 20, 17 to 20, 18 to 20, or 19 to 20 amino acid variations as compared to wild-type Slx9. In some embodiments, the non-natural flavin-dependent oxidase comprises about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, or about 100 amino acid variations as compared to wild-type Slx9. In some embodiments, the amino acid variation in the non-natural flavin-dependent oxidase is an amino acid substitution, deletion, or insertion. In some embodiments, the variation is a substitution of one or more amino acids in the wild-type Slx9 polypeptide sequence. In some embodiments, the variation is a deletion of one or more amino acids in the wild-type Slx9 polypeptide sequence. In some embodiments, the variation is an insertion of one or more amino acids in the wild-type Slx9 polypeptide sequence.

In some embodiments, the non-natural flavin-dependent oxidase comprising at least 70%, at least 80%, least at least 85%, at least 90% or at least 95% sequence identity to SEQ ID NO:18 comprises 1 to 100, 1 to 90, 1 to 80, 1 to 70, 1 to 60, 1 to 50, 1 to 40, 1 to 30, 1 to 25, 1 to 20, 2 to 20, 3 to 20, 4 to 20, 5 to 20, 6 to 20, 7 to 20, 8 to 20, 9 to 20, 10 to 20, 11 to 20, 12 to 20, 13 to 20, 14 to 20, 15 to 20, 16 to 20, 17 to 20, 18 to 20, or 19 to 20 amino acid variations as compared to wild-type Frx9. In some embodiments, the non-natural flavin-dependent oxidase comprises about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, or about 100 amino acid variations as compared to wild-type Frx9. In some embodiments, the amino acid variation in the non-natural flavin-dependent oxidase is an amino acid substitution, deletion, or insertion. In some embodiments, the variation is a substitution of one or more amino acids in the wild-type Frx9 polypeptide sequence. In some embodiments, the variation is a deletion of one or more amino acids in the wild-type Frx9 polypeptide sequence. In some embodiments, the variation is an insertion of one or more amino acids in the wild-type Frx9 polypeptide sequence.

In some embodiments, the non-natural flavin-dependent oxidase comprising at least 70%, at least 80%, least at least 85%, at least 90% or at least 95% sequence identity to SEQ ID NO:19 comprises 1 to 100, 1 to 90, 1 to 80, 1 to 70, 1 to 60, 1 to 50, 1 to 40, 1 to 30, 1 to 25, 1 to 20, 2 to 20, 3 to 20, 4 to 20, 5 to 20, 6 to 20, 7 to 20, 8 to 20, 9 to 20, 10 to 20, 11 to 20, 12 to 20, 13 to 20, 14 to 20, 15 to 20, 16 to 20, 17 to 20, 18 to 20, or 19 to 20 amino acid variations as compared to wild-type Kcx9. In some embodiments, the non-natural flavin-dependent oxidase comprises about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, or about 100 amino acid variations as compared to wild-type Kcx9. In some embodiments, the amino acid variation in the non-natural flavin-dependent oxidase is an amino acid substitution, deletion, or insertion. In some embodiments, the variation is a substitution of one or more amino acids in the wild-type Kcx9 polypeptide sequence. In some embodiments, the variation is a deletion of one or more amino acids in the wild-type Kcx9 polypeptide sequence. In some embodiments, the variation is an insertion of one or more amino acids in the wild-type Kcx9 polypeptide sequence.

In some embodiments, the non-natural flavin-dependent oxidase comprising at least 70%, at least 80%, least at least 85%, at least 90% or at least 95% sequence identity to any one of SEQ ID NOs:1-3, any one of SEQ ID NOs:10-13, or any one of SEQ ID NOs:17-19 is capable of converting CBGA to CBCA, THCA, CBDA, or a combination thereof. In some embodiments, the non-natural flavin-dependent oxidase converts CBGA to CBDA. In some embodiments, the non-natural flavin-dependent oxidase converts CBGA to THCA. In some embodiments, the non-natural flavin-dependent oxidase converts CBGA to CBCA. In some embodiments, the conversion is performed at about pH 4 to about pH 9. In some embodiments, the conversion is performed at about pH 4.5 to about pH 8.5. In some embodiments, the conversion is performed at about pH 5 to about pH 8. In some embodiments, the conversion is performed at about pH 5.5 to about pH 7.5. In some embodiments, the conversion is performed at about pH 4, about pH 4.5 about pH 5, about pH 5.5, about pH 6, about pH 6.5, about pH 7, about pH 7.5, about pH 8, about pH 8.5, or about pH 9. In some embodiments, the conversion is performed at about pH 5. In some embodiments, the conversion is performed at about pH 7.4 or about pH 7.5. In some embodiments, the non-natural flavin-dependent oxidase has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least or about 99%, or at least about 100% of the catalytic activity of a wild-type CBCAS. As referred to throughout the application, when comparing the catalytic activity of at least two enzymes, it will be understood by one of ordinary skill in the art that the enzymes can be subjected to the same or substantially the same reaction conditions or the enzymes can be subjected to the optimal reaction conditions for each enzyme, and catalytic activity is assessed using the same or substantially the same methods and/or equipment. Optimal reaction conditions for the enzymes described herein can be determined by one of ordinary skill in the art.

In some embodiments, the non-natural flavin-dependent oxidase comprising at least 70%, at least 80%, least at least 85%, at least 90% or at least 95% sequence identity to any one of SEQ ID NOs:1-3, any one of SEQ ID NOs:10-13, or any one of SEQ ID NOs:17-19 converts CBGA to CBCA at about pH 4 to about pH 9. In some embodiments, the non-natural flavin-dependent oxidase converts CBGA to CBCA at about pH 4.5 to about pH 8.5. In some embodiments, the non-natural flavin-dependent oxidase converts CBGA to CBCA at about pH 5 to about pH 8. In some embodiments, the non-natural flavin-dependent oxidase converts CBGA to CBCA at about pH 5.5 to about pH 7.5. In some embodiments, the non-natural flavin-dependent oxidase converts CBGA to CBCA at about pH 4, about pH 4.5 about pH 5, about pH 5.5, about pH 6, about pH 6.5, about pH 7, about pH 7.5, about pH 8, about pH 8.5, or about pH 9. In some embodiments, the non-natural flavin-dependent oxidase converts CBGA to CBCA at about pH 5. In some embodiments, the non-natural flavin-dependent oxidase converts CBGA to CBCA at about pH 7.4 or about pH 7.5. In some embodiments, the non-natural flavin-dependent oxidase comprises at least 70%, at least 80%, least at least 85%, at least 90% or at least 95% sequence identity to any one of SEQ ID NOs:1-3, SEQ ID NO:10, SEQ ID NO:11, SEQ ID NO:13, or SEQ ID NO:19.

In some embodiments, the non-natural flavin-dependent oxidase comprising at least 70%, at least 80%, least at least 85%, at least 90% or at least 95% sequence identity to any one of SEQ ID NOs:1-3, any one of SEQ ID NOs:10-13, or any one of SEQ ID NOs:17-19 is further capable of converting CBGOA to CBCOA, CBDOA, THCOA, or a combination thereof. In some embodiments, the non-natural flavin-dependent oxidase converts CBGOA to CBDOA. In some embodiments, the non-natural flavin-dependent oxidase converts CBGOA to THCOA. In some embodiments, the non-natural flavin-dependent oxidase converts CBGOA to CBCOA. In some embodiments, the non-natural flavin-dependent oxidase comprising at least 70%, at least 80%, least at least 85%, at least 90% or at least 95% sequence identity to any one of SEQ ID NOs:1-3, any one of SEQ ID NOs:10-13, or any one of SEQ ID NOs:17-19 is further capable of converting CBGVA to CBCVA, CBDVA, THCVA, or a combination thereof. In some embodiments, the non-natural flavin-dependent oxidase converts CBGVA to CBDVA. In some embodiments, the non-natural flavin-dependent oxidase further converts CBGVA to THCVA. In some embodiments, the non-natural flavin-dependent oxidase further converts CBGVA to CBCVA. In some embodiments, the non-natural flavin-dependent oxidase comprising at least 70%, at least 80%, least at least 85%, at least 90% or at least 95% sequence identity to any one of SEQ ID NOs:1-3, any one of SEQ ID NOs:10-13, or any one of SEQ ID NOs:17-19 is further capable of converting CBG to CBC, CBD, THC, or a combination thereof. In some embodiments, the non-natural flavin-dependent oxidase converts CBG to CBD. In some embodiments, the non-natural flavin-dependent oxidase converts CBG to THC. In some embodiments, the non-natural flavin-dependent oxidase converts CBG to CBC. In some embodiments, the non-natural flavin-dependent oxidase comprising at least 70%, at least 80%, least at least 85%, at least 90% or at least 95% sequence identity to any one of SEQ ID NOs:1-3, any one of SEQ ID NOs:10-13, or any one of SEQ ID NOs:17-19 is further capable of converting CBGO to CBCO, CBDO, THCO, or a combination thereof. In some embodiments, the non-natural flavin-dependent oxidase converts CBGO to CBDO. In some embodiments, the non-natural flavin-dependent oxidase converts CBGO to THCO. In some embodiments, the non-natural flavin-dependent oxidase converts CBGO to CBCO. In some embodiments, the non-natural flavin-dependent oxidase comprising at least 70%, at least 80%, least at least 85%, at least 90% or at least 95% sequence identity to any one of SEQ ID NOs:1-3, any one of SEQ ID NOs:10-13, or any one of SEQ ID NOs:17-19 is further capable of converting CBGV to CBCV, CBDV, THCV, or a combination thereof. In some embodiments, the non-natural flavin-dependent oxidase converts CBGV to CBDV. In some embodiments, the non-natural flavin-dependent oxidase further converts CBGV to THCV. In some embodiments, the non-natural flavin-dependent oxidase further converts CBGV to CBCV.

In some embodiments, the conversion is performed at about pH 4 to about pH 9. In some embodiments, the conversion is performed at about pH 4.5 to about pH 8.5. In some embodiments, the conversion is performed at about pH 5 to about pH 8. In some embodiments, the conversion is performed at about pH 5.5 to about pH 7.5. In some embodiments, the conversion is performed at about pH 4, about pH 4.5 about pH 5, about pH 5.5, about pH 6, about pH 6.5, about pH 7, about pH 7.5, about pH 8, about pH 8.5, or about pH 9. In some embodiments, the conversion is performed at about pH 5. In some embodiments, the conversion is performed at about pH 7.4 or about pH 7.5.

In some embodiments, the non-natural flavin-dependent oxidase provided herein further comprises an affinity tag, a purification tag, a solubility tag, or a combination thereof. As used in the context of proteins and polypeptides, a “tag” can refer to a short polypeptide sequence, typically about 5 to about 50 amino acids in length, that is covalently attached to the protein of interest, e.g., the non-natural flavin-dependent oxidase. Additionally or alternatively, a tag can also comprise a polypeptide that is greater than 50 amino acids in length and that provides a desired property, e.g., increases solubility, to the tagged protein of interest. In some embodiments, the tag is attached to the protein such that it in the same reading frame as the protein, i.e., “in-frame.” In general, the tag allows a specific chemical or enzymatic modification to the protein of interest. Solubility tags increases the solubility of the tagged protein and include, e.g., thioredoxin (TRX), poly(NANP), maltose-binding protein (MBP), and glutathione S-transferase (GST). Affinity tags allow the protein to bind to a specific molecule. Examples of affinity tags include chitin binding protein (CBP), Strep-tag, poly(His) tag, and the like; in addition, certain solubility tags such as MBP and GST can also serve as an affinity tag. Purification tags, also termed chromatography tags, allow the protein to be separated from other components in a particular purification or separation technique and are typically comprise polyanionic amino acids, such as the FLAG-tag. Further examples of tags that can be included on the non-natural flavin-dependent oxidases provided herein include, without limitation, epitope tags such as ALFA-tag, V5-tag, Myc-tag, HA-tag, Spot-tag, T7-tag, and NE-tag, which can be useful in western blotting or immunoprecipitation; and fluorescence tags such as GFP and its variants for visualization of the tagged protein. One of ordinary skill in the art would understand that the non-natural flavin-dependent oxidase provided herein can comprise a single tag, or a combination of tags including multiple functions. Methods of producing tagged proteins, e.g., a tagged non-natural flavin-dependent oxidase, are known in the field. See, e.g., Kimple et al. (2013), Curr Protoc Protein Sci 73: Unit-9.9.

In some embodiments, the disclosure further provides a polynucleotide comprising a nucleic acid sequence encoding the non-natural flavin-dependent oxidase described herein. In some embodiments, the disclosure further provides a polynucleotide comprising: (a) a nucleic acid sequence encoding a polypeptide comprising at least 80% sequence identity to any one of SEQ ID NOs:1-3, any one of SEQ ID NOs:10-13, or any one of SEQ ID NOs:17-19; and (b) a heterologous regulatory element operably linked to the nucleic acid sequence. In some embodiments, the nucleic acid sequence encodes a polypeptide comprising at least 85%, at least 90%, at least 91%, at least 92%, at least 93%, at least 94%, at least 95%, at least 96%, at least 97%, at least 98%, at least 99%, or 100% sequence identity to SEQ ID NO:1. In some embodiments, the nucleic acid sequence encodes a polypeptide comprising at least 85%, at least 90%, at least 91%, at least 92%, at least 93%, at least 94%, at least 95%, at least 96%, at least 97%, at least 98%, at least 99%, or 100% sequence identity to SEQ ID NO:2. In some embodiments, the nucleic acid sequence encodes a polypeptide comprising at least 85%, at least 90%, at least 91%, at least 92%, at least 93%, at least 94%, at least 95%, at least 96%, at least 97%, at least 98%, at least 99%, or 100% sequence identity to SEQ ID NO:3. In some embodiments, the nucleic acid sequence encodes a polypeptide comprising at least 85%, at least 90%, at least 91%, at least 92%, at least 93%, at least 94%, at least 95%, at least 96%, at least 97%, at least 98%, at least 99%, or 100% sequence identity to SEQ ID NO:10. In some embodiments, the nucleic acid sequence encodes a polypeptide comprising at least 85%, at least 90%, at least 91%, at least 92%, at least 93%, at least 94%, at least 95%, at least 96%, at least 97%, at least 98%, at least 99%, or 100% sequence identity to SEQ ID NO:11. In some embodiments, the nucleic acid sequence encodes a polypeptide comprising at least 85%, at least 90%, at least 91%, at least 92%, at least 93%, at least 94%, at least 95%, at least 96%, at least 97%, at least 98%, at least 99%, or 100% sequence identity to SEQ ID NO:12. In some embodiments, the nucleic acid sequence encodes a polypeptide comprising at least 85%, at least 90%, at least 91%, at least 92%, at least 93%, at least 94%, at least 95%, at least 96%, at least 97%, at least 98%, at least 99%, or 100% sequence identity to SEQ ID NO:13. In some embodiments, the nucleic acid sequence encodes a polypeptide comprising at least 85%, at least 90%, at least 91%, at least 92%, at least 93%, at least 94%, at least 95%, at least 96%, at least 97%, at least 98%, at least 99%, or 100% sequence identity to SEQ ID NO:17. In some embodiments, the nucleic acid sequence encodes a polypeptide comprising at least 85%, at least 90%, at least 91%, at least 92%, at least 93%, at least 94%, at least 95%, at least 96%, at least 97%, at least 98%, at least 99%, or 100% sequence identity to SEQ ID NO:18. In some embodiments, the nucleic acid sequence encodes a polypeptide comprising at least 85%, at least 90%, at least 91%, at least 92%, at least 93%, at least 94%, at least 95%, at least 96%, at least 97%, at least 98%, at least 99%, or 100% sequence identity to SEQ ID NO:19.

In some embodiments, the disclosure further provides a polynucleotide comprising SEQ ID NO:4, SEQ ID NO:5, SEQ ID NO:9, or any one of SEQ ID NOs:14-16. In some embodiments, the polynucleotide comprises a nucleic acid sequence comprising at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:4, SEQ ID NO:5, SEQ ID NO:9, any one of SEQ ID NOs:14-16, or any one of SEQ ID NOs:20-23.

In some embodiments, the nucleic acid sequence encoding the non-natural flavin-dependent oxidase is codon optimized. An example of a codon optimized sequence is, in one instance, a sequence optimized for expression in a bacterial host cell, e.g., E. coli. In some embodiments, one or more codons in a nucleic acid sequence encoding the non-natural flavin-dependent oxidase described herein corresponds to the most frequently used codon for a particular amino acid in the bacterial host cell.

In some embodiments, the heterologous regulatory element of the polynucleotide comprises a promoter, an enhancer, a silencer, a response element, or a combination thereof. In some embodiments, the heterologous regulatory element of (b) is a bacterial regulatory element. Non-limiting examples of bacterial regulatory elements include the T7 promoter, Sp6 promoter, lac promoter, araBad promoter, trp promoter, and Ptac promoter. Further examples of regulatory elements can be found, e.g., using the PRODORIC2 database (Eckweiler et al. (2018), Nucleic Acids Res 46(D1): D320-D326).

In some embodiments, the disclosure provides an expression construct comprising the polynucleotide provided herein. Expression constructs are described herein and include, e.g., pQE vectors (Qiagen), pBluescript plasmids, pNH vectors, lambda-ZAP vectors (Stratagene); pTrc99a, pKK223-3, pDR540, and pRIT2T (Pharmacia). In some embodiments, the expression construct comprises a regulatory element. Regulatory elements are provided herein.

In some embodiments, the disclosure provides an engineered cell comprising the non-natural flavin-dependent oxidase described herein, the polynucleotide described herein, the expression construct comprising the polynucleotide described herein, or a combination thereof. In some embodiments, the engineered cell comprises the non-natural flavin-dependent oxidase. In some embodiments, the engineered cell comprises the polynucleotide comprising a nucleic acid sequence encoding the non-natural flavin-dependent oxidase. In some embodiments, the engineered cell comprises the polynucleotide comprising (a) a nucleic acid sequence encoding a polypeptide comprising at least 80% sequence identity to any one of SEQ ID NOs:1-3, any one of SEQ ID NOs:10-13, or any one of SEQ ID NOs:17-19; and (b) a heterologous regulatory element operably linked to the nucleic acid sequence. In some embodiments, the engineered cell comprises the expression construct comprising the polynucleotide provided herein. In some embodiments, the disclosure provides a method of making an isolated non-natural flavin-dependent oxidase, comprising isolating the non-natural flavin-dependent oxidase expressed in the engineered cell provided herein. In some embodiments, the disclosure provides an isolated non-natural flavin-dependent oxidase, wherein the isolated non-natural flavin-dependent oxidase is expressed and isolated from the engineered cell.

In some embodiments, the engineered cell further comprises a cannabinoid biosynthesis pathway enzyme. An exemplary cannabinoid biosynthesis pathway starts from the conversion of hexanoate to hexanoyl-CoA (Hex-CoA) via hexanoyl-CoA synthetase. Hex-CoA is then converted to 3-oxooctanoyl-CoA, then 3,5-dioxodecanoyl-CoA, then 3,5,7-trioxododecanoyl-CoA by olivetol synthase (OLS; also known as tetraketide synthase or TKS), which is subsequently converted to olivetolic acid by olivetolic acid cyclase (OAC). A prenyltransferase then catalyzes the reaction between olivetolic acid and geranyldiphosphate (GPP) to produce CBGA, which can be converted to CBG via non-enzymatic decarboxylation. In an analogous manner, CBGOA is produced from the prenyltransferase-catalyzed reaction between orsellinic acid and GPP; CBGVA is produced from the prenyltransferase-catalyzed reaction between divarinic acid and GPP. In some embodiments, the CBGA, CBG, CBGOA, and/or CBGVA produced from the cannabinoid biosynthesis pathways are further converted into a cannabinoid by the non-natural flavin-dependent oxidases provided herein. Cannabinoid biosynthesis pathways are further described, e.g., in Degenhardt et al., Chapter 2—The Biosynthesis of Cannabinoids. Handbook of Cannabis and Related Pathologies, pp. 13-23; Elsevier Academic Press, 2017. In some embodiments, the cannabinoid biosynthesis pathway enzyme comprises an enzyme from Cannabis sativa, e.g., olivetol synthase (OLS), olivetolic acid cyclase (OAC), a geranyl pyrophosphate (GPP) pathway enzyme, and/or prenyltransferase. In some embodiments, the cannabinoid biosynthesis pathway enzyme comprises a homolog of a C. sativa enzyme, e.g., a homolog of OLS, OAC, GPP pathway enzyme, and/or prenyltransferase. It will be understood by one of ordinary skill in the art that a homolog of a cannabinoid biosynthesis pathway enzyme can be a sequence homolog, a structural homolog, and/or an enzyme activity homolog.

In some embodiments, the engineered cell produces CBCA, CBDA, THCA, CBCOA, CBDOA, THCOA, CBCVA, CBDVA, THCVA, CBC, CBD, THC, CBCO, CBDO, THCO, CBCV, CBDV, and/or THCV. In some embodiments, the engineered cell produces CBCA, CBCOA, CBCVA, CBC, CBCO, and/or CBCV. In some embodiments, the engineered cell produces CBCA. In some embodiments, the engineered cell produces CBDA. In some embodiments, the engineered cell produces THCA.

In some embodiments, the engineered cell further comprises an enzyme in the CBGA biosynthesis pathway. In some embodiments, the engineered cell further comprises an enzyme in the CBG biosynthesis pathway. In some embodiments, the engineered cell comprises an enzyme in the CBGOA biosynthesis pathway. In some embodiments, the engineered cell comprises an enzyme in the CBGVA biosynthesis pathway. In some embodiments, the engineered cell comprises an enzyme in the CBGO biosynthesis pathway. In some embodiments, the engineered cell comprises an enzyme in the CBGV biosynthesis pathway. In some embodiments, the cannabinoid biosynthesis pathway enzyme of the engineered cell comprises OLS, OAC, prenyltransferase, or a combination thereof.

In some embodiments, CBGA is produced from olivetolic acid (OA) and geranyldiphosphate (GPP). In some embodiments, CBG is produced from CBGA. In some embodiments, CBGOA is produced from orsellinic acid (OSA) and GPP. In some embodiments, CBGVA is produced from divarinic acid (DA) and GPP. In some embodiments, the engineered cells of the disclosure have higher levels of available GPP, OA, OSA, DA, CBGA, CBG, CBGOA, and/or CBGVA (and derivatives or analogs thereof) as compared to a naturally-occurring, non-engineered cell.

### OLS

In some embodiments, the engineered cell of the disclosure further comprises an enzyme in the olivetolic acid pathway. In some embodiments, the enzyme in the olivetolic acid pathway is olivetol synthase (OLS). OLS catalyzes the addition of two malonyl-CoA (Mal-CoA) and hexanoyl-CoA (Hex-CoA) to form 3,5-dioxodecanoyl-CoA, which can be further converted by OLS to 3,5,7-trioxododecanoyl-CoA with the addition of a third Mal-CoA. 3,5,7-trioxododecanoyl-CoA can subsequently be converted to OA by OAC.

Although the metabolic pathway is discussed herein with reference to certain precursors and intermediates, it is understood that analogs may be substituted in essentially the same reactions. For example, it is understood that Hex-CoA analogs, including other acyl-CoAs, can be used in place of Hex-CoA. Exemplary analogs include, but are not limited to any C₂-C₂₀ acyl-CoA such as acetyl-CoA, propionyl-CoA, butyryl-CoA, pentanoyl-CoA, heptanoyl-CoA, octanoyl-CoA, nonanoyl-CoA, decanoyl-CoA, and aromatic acid CoA such as benzoic, chorismic, phenylacetic, and phenoxyacetic acid-CoA.

In some embodiments, the engineered cells of the disclosure have increased production of one or more precursors (e.g., Mal-CoA, Hex-CoA or other acyl-CoA, OA, OSA, DA, CBGA, CBGOA, and/or CBGVA) of the cannabinoids provided herein, e.g., CBCA, CBDA, THCA, CBCOA, CBDOA, THCOA, CBCVA, CBDVA, THCVA, CBC, CBD, THC, CBCO, CBDO, THCO, CBCV, CBDV, and/or THCV. In some embodiments, the engineered cells of the disclosure have increased production of one or more precursors (e.g., Mal-CoA, Hex-CoA or other acyl-CoA, OA, OSA, DA, CBGA, CBGOA, and/or CBGVA) of THCA, CBCA, CBCOA, CBCVA, CBC, CBCO, and/or CBCV.

In some embodiments, the engineered cells of the disclosure have increased production of OA precursors, e.g., Mal-CoA and/or acyl-CoA (such as, e.g., Hex-CoA or any other acyl-CoA described herein). In some embodiments, the non-natural OLS preferentially catalyzes the condensation of Mal-CoA and acyl-CoA (such as, e.g., Hex-CoA or any other acyl-CoA described herein) to form a polyketide (such as, e.g., 3,5,7-trioxododecanoyl-CoA and 3,5,7-trioxododecanoate and their analogs) over the reaction side products, e.g., pentyl diacetic acid lactone (PDAL), hexanoyl triacetic acid lactone (HTAL), or other lactone analogs compared with a wild-type OLS.

In some embodiments, the engineered cell expresses an exogenous or overexpresses an exogenous or endogenous OLS. In some embodiments, the OLS is a natural OLS, e.g., a wild-type OLS. In some embodiments, the OLS is a non-natural OLS. In some embodiments, the OLS comprises one or more amino acid substitutions relative to a wild-type OLS. In some embodiments, the one or more amino acid substitutions in the non-natural OLS increases the activity of the OLS as compared to a wild-type OLS.

In some embodiments, the OLS has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:6.

In some embodiments, the OLS comprises a variation at amino acid position A125, S126, D185, M187, L190, G204, G209, D210, G211, G249, G250, L257, F259, M331, S332, or a combination thereof, wherein the position corresponds to SEQ ID NO:6. In some embodiments, the variation is an amino acid substitution. OLS and non-natural variants thereof are further discussed in, e.g., WO2020/214951.

In some embodiments, the non-natural OLS comprises an amino acid substitution selected from A125G, A125S, A125T, A125C, A125Y, A125H, A125N, A125Q, A125D, A125E, A125K, A125R, S126G, S126A, D185G, D185G, D185A, D185S, D185P, D185C, D185T, D185N, M187G, M187A, M187S, M187P, M187C, M187T, M187D, M187N, M187E, M187Q, M187H, M187H, M187V, M187L, M187I, M187K, M187R, L190G, L190A, L190S, L190P, L190C, L190T, L190D, L190N, L190E, L190Q, L190H, L190V, L190M, L1901, L190K, L190R, G204A, G204C, G204P, G204V, G204L, G204I, G204M, G204F, G204W, G204S, G204T, G204Y, G204H, G204N, G204Q, G204D, G204E, G204K, G204R, G209A, G209C, G209P, G209V, G209L, G209I, G209M, G209F, G209W, G209S, G209T, G209Y, G209H, G209N, G209Q, G209D, G209E, G209K, G209R, D210A, D210C, D210P, D210V, D210L, D210I, D210M, D210F, D210W, D210S, D210T, D210Y, D210H, D210N, D210Q, D210E, D210K, D210R, G211A, G211C, G211P, G211V, G211L, G2111, G211M, G211F, G211W, G211S, G211T, G211Y, G211H, G211N, G211Q, G211D, G211E, G211K, G211R, G249A, G249C, G249P, G249V, G249L, G249I, G249M, G249F, G249W, G249S, G249T, G249Y, G249H, G249N, G249Q, G249D, G249E, G249K, G249R, G249S, G249T, G249Y, G250A, G250C, G250P, G250V, G250L, G250I, G250M, G250F, G250W, G250S, G250T, G250Y, G250H, G250N, G250Q, G250D, G250E, G250K, G250R, L257V, L257M, L257I, L257K, L257R, L257F, L257Y, L257W, L257S, L257T, L257C, L257H, L257N, L257Q, L257D, L257E, F259G, F259A, F259C, F259P, F259V, F259L, F259I, F259M, F259Y, F259W, F259S, F259T, F259Y, F259H, F259N, F259Q, F259D, F259E, F259K, F259R, M331G, M331A, M331S, M331P, M331C, M331T, M331D, M331N, M331E, M331Q, M331H, M331V, M331L, M3311, M331K, M331R, S332G, S332A, or a combination thereof, wherein the position corresponds to SEQ ID NO:6.

In some embodiments, the disclosure provides a composition comprising the non-natural flavin-dependent oxidase described herein and the OLS described herein. In some embodiments, the disclosure provides an engineered cell comprising the non-natural flavin-dependent oxidase described herein and the OLS described herein. In some embodiments, the disclosure provides one or more polynucleotides comprising one or more nucleic acid sequences encoding the non-natural flavin-dependent oxidase described herein and the OLS described herein. In some embodiments, the OLS is a non-natural OLS. In some embodiments, the disclosure provides an expression construct comprising the one or more polynucleotides. In some embodiments, the expression construct comprises a single expression vector. In some embodiments, the expression construct comprises more than one expression vector. In some embodiments, the disclosure provides an engineered cell comprising the one or more polynucleotides. In some embodiments, the disclosure provides an engineered cell comprising the expression construct. In some embodiments, the engineered cell produces CBCA, CBDA, THCA, CBCOA, CBDOA, THCOA, CBCVA, CBDVA, THCVA, CBCO, CBDO, THCO, CBCV, CBDV, THCV, CBC, CBD, and/or THC. In some embodiments, the engineered cell produces CBCA, CBCOA, CBCVA, CBCO, CBCV, and/or CBC. In some embodiments, the engineered cell produces CBCA.

### OAC

In some embodiments, the engineered cell of the disclosure further comprises an enzyme in the olivetolic acid pathway. In some embodiments, the enzyme in the olivetolic acid pathway is olivetolic acid cyclase (OAC). As discussed herein, OAC catalyzes the conversion of 3,5,7-trioxododecanoyl-CoA to OA.

In some embodiments, the engineered cell expresses an exogenous or overexpresses an exogenous or endogenous OAC. In some embodiments, the OAC is a natural OAC, e.g., a wild-type OAC. In some embodiments, the OAC is a non-natural OAC. In some embodiments, the OAC comprises one or more amino acid substitutions relative to a wild-type OAC. In some embodiments, the one or more amino acid substitutions in the non-natural OAC increases the activity of the OAC as compared to a wild-type OAC. OAC and non-natural variants thereof are further discussed in, e.g., WO2020/247741.

In some embodiments, the OAC has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:7.

In some embodiments, the OAC comprises a variation at amino acid position L9, F23, V59, V61, V66, E67, I69, Q70, I73, I74, V79, G80, F81, G82, D83, R86, W89, L92, or 194, V46, T47, Q48, K49, N50, K51, V46, T47, Q48, K49, N50, K51, or a combination thereof, wherein the position corresponds to SEQ ID NO:7. In some embodiments, the variation is an amino acid substitution. In some embodiments, the variation is in a first peptide (e.g., a first monomer) of an OAC dimer. In some embodiments, the variation is in a second peptide (e.g., a second monomer) of an OAC dimer.

In some embodiments, the OAC forms a dimer, wherein a first peptide of the dimer (e.g., a first monomer) of the dimer comprises a variation at amino acid position H5, I7, L9, F23, F24, Y27, V59, V61, V66, E67, 169, Q70, I73, I74, V79, G80, F81, G82, D83, R86, W89, L92, 194, D96, V46, T47, Q48, K49, N50, K51, or combination thereof, and wherein a second peptide (e.g., a second monomer) of the dimer comprises a variation at amino acid position V46, T47, Q48, K49, N50, K51, or combination thereof, wherein the position corresponds to SEQ ID NO:7. In some embodiments, the OAC forms a dimer, wherein a first peptide of the dimer comprises a variation at amino acid position L9, F23, V59, V61, V66, E67, I69, Q70, I73, I74, V79, G80, F81, G82, D83, R86, W89, L92, 194, V46, T47, Q48, K49, N50, K51, or combination thereof, and a second peptide of the dimer comprises a variation at amino acid position V46, T47, Q48, K49, N50, K51, or combination thereof, wherein the position corresponds to SEQ ID NO:7.

In some embodiments, the OAC comprises an amino acid substitution selected from H5X¹, wherein X¹ is G, A, C, P, V, L, I, M, F, Y, W, Q, E, K, R, S, T, Y, N, Q, D, E, K, or R; I7X², wherein X² is G, A, C, P, V, L, M, F, Y, W, K, R, S, T, H, N, Q, D, or E; L9X³, wherein X³ is G, A, C, P, V, I, M, F, Y, W, K, R, S, T, Y, H, N, Q, D, E, K, or R; F23X⁴, wherein X⁴ is G, A, C, P, V, L, I, M, Y, W, S, T, H, N, Q, D, E, K, or R; F24X⁵, wherein X⁵ is G, A, C, P, V, I, M, Y, S, T, H, N, Q, D, E, K, R, or W; Y27X⁶, wherein X⁶ is G, A, C, P, V, L, I, M, F, W, S, T, H, N, Q, D, E, K, or R; V59X⁷, wherein X⁷ is G, A, C, P, L, I, M, F, Y, W, H, Q, E, K, or R; V61X⁸, wherein X⁸ is G, A, C, P, L, I, M, F, Y, W, H, Q, E, K, R, S, T, N, or D; V66X⁹, wherein X⁹ is G, A, C, P, L, I, M, F, Y, or W; E67X¹⁰, wherein X¹⁰ is G, A, C, P, V, L, I, M, F, Y, or W; 169X¹¹, wherein X¹¹ is G, A, C, P, V, L, M, F, Y, or W; Q70X¹², wherein X¹² is S, T, H, N, D, E, R, K, or Y; I73X¹³ wherein X¹³ is G, A, C, P, V, L, M, F, Y, or W; 174X¹⁴, wherein X¹⁴ is G, A, C, P, V, L, M, F, Y, or W; V79X¹⁵, wherein X¹⁵ is G, A, C, P, L, I, M, F, Y, or W; G80X¹⁶, wherein X¹⁶ is A, C, P, V, L, I, M, F, Y, W, S, T, H, N, Q, D, E, K, or R; F81X¹⁷, wherein X¹⁷ is G, A, C, P, V, L, I, M, Y, W, S, T, H, N, Q, D, E, R, or K; G82X¹⁸, wherein X¹⁸ is A, C, P, V, L, I, M, F, Y, W, S, T, H, N, Q, E, K, or R; D83X¹⁹, wherein X¹⁹ is S, T, H, Q, N, E, R, K, or Y; R86X²⁰, wherein X²⁰ is S, T, H, Q, N, D, E, K, or Y; W89X²¹, wherein X²¹ is G, A, C, P, V, L, I, M, F, Y, W, S, T, H, N, Q, D, E, K, or R; L92X²², wherein X²² is G, A, C, P, V, I, M, F, Y, or W; I94X²³, wherein X²³ is G, A, C, P, V, L, M, F, Y, W, K, R, S, T, Y, H, N, Q, D, or E; D96X²⁴, wherein X²⁴ is S, T, H, Q, N, E, R, K, or Y; V46X²⁵, wherein X²⁵ is G, A, C, P, L, I, M, F, Y, or W; T47X²⁶, wherein X²⁶ is S, H, Q, N, D, E, R, K, or Y; Q48X²⁷, wherein X²⁷ is S, T, H, N, D, E, R, K, or Y; K49X²⁸, wherein X²⁸ is S, T, H, Q, N, D, E, R, or Y; N50X²⁹, wherein X²⁹ is G, A, C, P, V, L, I, M, F, Y, or W; K51X³⁰, wherein X³⁰ is S, T, H, Q, N, D, E, R, or Y; V46*X³¹, wherein X³¹ is G, A, C, P, L, I, M, F, Y, or W; T47*X³², wherein X³² is S, H, Q, N, D, E, R, K, or Y; Q48*X³³, wherein X³³ is S, T, H, N, D, E, R, K, or Y; K49*X³⁴, wherein X³⁴ is S, T, H, Q, N, D, E, R, or Y; N50*X³⁵, wherein X³⁵ is G, A, C, P, V, L, I, M, F, Y, or W; K51*X³⁶, wherein X³⁶ is S, T, H, Q, N, D, E, R, or Y; and a combination thereof; wherein the amino acid position corresponds to SEQ ID NO:7, and wherein the “*” following the amino acid position indicates amino acid residues from a second peptide of a OAC dimer (e.g., monomer B) and corresponding to SEQ ID NO:7.

In some embodiments, the OAC comprises more than one amino acid variations. In some embodiments, the OAC is not a single substitution at position K4A, H5A, H5L, H5Q, H5S, H5N, H5D, I7L, I7F, L9A, L9W, K12A, F23A, F23I, F23W, F23L, F24L, F24W, F24A, Y27F, Y27M, Y27W, V28F, V29M, K38A, V40F, D45A, H57A, V59M, V59A, V59F, Y72F, H75A, H78A, H78N, H78Q, H78S, H78D, or D96A, wherein the amino acid position corresponds to SEQ ID NO:7.

In some embodiments, the OAC described herein is capable of producing olivetolic acid at a faster rate compared with a wild-type OAC. In some embodiments, the OAC has increased affinity for a polyketide (e.g., 3,5,7-trioxododecanoyl-CoA or an analog thereof, as produced by an OLS described herein) compared with a wild-type OAC. In some embodiments, the rate of formation of olivetolic acid from 3,5,7-trioxododecanoyl-CoA or analog thereof by the OAC described herein is about 1.2 times to about 300 times, about 1.5 times to about 200 times, or about 2 times to about 30 times as compared to a wild-type OAC. The rate of formation of olivetolic acid from 3,5,7-trioxododecanoyl-CoA or an analog thereof can be determined in an in vitro enzymatic reaction using a purified OAC. Methods of determining enzyme kinetics and product formation rate are known in the field.

In some embodiments, the OLS described herein is enzymatically capable of at least about 1.1, 1.2, 1.5, 2, 3, 4, 5, 6, 7, 8, 9, 10, 12, 15, 20, or greater rate of formation of OA and/or olivetol from Mal-CoA and Hex-CoA in the presence of an excess of the OAC described herein, as compared to a wild type OLS.

In some embodiments, the OAC is present in molar excess of the OLS in the engineered cell. In some embodiments, the molar ratio of the OLS to the OAC is about 1:1.1, 1:1.2, 1:1.5, 1:1.8, 1:2, 1:3, 1:4, 1:5, 1:10, 1:20, 1:25, 1:50, 1:75, 1:100, 1:125, 1:150, 1:200, 1:250, 1:300, 1:350, 1:400, 1:450, 1:500, 1:1000, 1:1250, 1:1500, 1:2000, 1:2500, 1:5000, 1:7500, 1:10,000, or 1 to more than 10,000. In some embodiments, the molar ratio of the OLS to the OAC is about 1000:1, 500:1, 100:1, 10:1, 5:1, 2.5:1. 1.5:1, 1.2:1. 1.1:1, 1:1, or less than 1 to 1. In some embodiments, the enzyme turnover rate of the OAC is greater than OLS. As used herein, “turnover rate” refers to the rate at which an enzyme can catalyze a reaction (e.g., turn substrate into product). In some embodiments, the higher turnover rate of OAC compared to OLS provides a greater rate of formation of OA than olivetol.

In some embodiments, the total byproducts (e.g., olivetol and analogs thereof, PDAL, HTAL, and other lactone analogs) of the OLS reaction products in the presence of molar excess of OAC, are in an amount (w/w) of less than about 50%, 45%, 40%, 35%, 30%, 25%, 20%, 15%, 12.5%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, 0.025%, or 0.01% of the total weight of the products formed by the combination of individual OLS and OAC enzyme reactions.

In some embodiments, the disclosure provides a composition comprising the non-natural flavin-dependent oxidase described herein and one or both of the OLS described herein and the OAC described herein. In some embodiments, the disclosure provides an engineered cell comprising the non-natural flavin-dependent oxidase described herein and one or both of the OLS described herein and the OAC described herein. In some embodiments, the disclosure provides one or more polynucleotides comprising one or more nucleic acid sequences encoding the non-natural flavin-dependent oxidase described herein and one or both of the OLS described herein and the OAC described herein. In some embodiments, the disclosure provides an expression construct comprising the one or more polynucleotides. In some embodiments, the expression construct comprises a single expression vector. In some embodiments, the expression construct comprises more than one expression vector. In some embodiments, the disclosure provides an engineered cell comprising the one or more polynucleotides. In some embodiments, the disclosure provides an engineered cell comprising the expression construct. In some embodiments, the engineered cell produces CBCA, CBDA, THCA, CBCOA, CBDOA, THCOA, CBCVA, CBDVA, THCVA, CBCO, CBDO, THCO, CBCV, CBDV, THCV, CBC, CBD, and/or THC. In some embodiments, the engineered cell produces CBCA, CBCOA, CBCVA, and/or CBC. In some embodiments, the engineered cell produces CBCA.

### GPP

In some embodiments, the engineered cell of the disclosure further comprises an enzyme in the geranyl pyrophosphate (GPP) pathway. GPP pathways are further provided, e.g., in WO 2017/161041. In some embodiments, the GPP pathway comprises a mevalonate (MVA) pathway, a non-mevalonate methylerythritol-4-phosphate (MEP) pathway, an alternative non-MEP, non-MVA geranyl pyrophosphate pathway, or a combination thereof. In some embodiments, the GPP pathway comprises an enzyme selected from geranyl pyrophosphate (GPP) synthase, famesyl pyrophosphate synthase, isoprenyl pyrophosphate synthase, geranylgeranyl pyrophosphate synthase, alcohol kinase, alcohol diphosphokinase, phosphate kinase, isopentenyl diphosphate isomerase, or a combination thereof. In some embodiments, the alternative non-MEP, non-MVA geranyl pyrophosphate pathway comprises alcohol kinase, alcohol diphosphokinase, phosphate kinase, isopentenyl disphosphate isomerase, geranyl pyrophosphate synthase, or a combination thereof.

GPP and its precursors may be produced from several pathways within a host cell, including the mevalonate pathway (MVA) or a non-mevalonate, methylerythritol-4-phosphate (MEP) pathway (also known as the deoxyxylulose-5-phosphate pathway), which produce isopentenyl pyrophosphate (IPP) and dimethylallyl pyrophosphate (DMAPP), which are isomerized by isopentenyl-diphosphate delta-isomerase (IDI) and converted GPP using geranyl pyrophosphate synthase (GPPS). As described herein, prenyltransferase can convert GPP and OA into CBGA, which can then be converted into CBCA, CBDA, and/or THCA by the non-natural flavin-dependent oxidase described herein. Prenyltransferase can also convert GPP and OSA into CBGOA, which can then be converted in CBCOA by the non-natural flavin-dependent oxidase described herein. Prenyltransferase can further convert GPP and DA into CBGVA, which can then be converted into CBCVA by the non-natural flavin-dependent oxidase described herein.

In some embodiments, the engineered cell produces GPP from a MVA pathway. In some embodiments, the engineered cell produces GPP from a MEP pathway. In some embodiments, the engineered cell expresses an exogenous or overexpresses an exogenous or endogenous gene that encodes any one of the enzymes in the MVA pathway or the MEP pathway, thereby increasing the production of GPP. In some embodiments, the MVA pathway enzyme is acetoacetyl-CoA thiolase (AACT); HMG-CoA synthase (HMGS); HMG-CoA reductase (HMGR); mevalonate-3-kinase (MVK); phosphomevalonate kinase (PMK); mevalonate-5-pyrophosphate decarboxylase (MVD); isopentenyl pyrophosphate isomerase (IDI), or geranyl pyrophosphate synthase (GPPS). In some embodiments, the MEP pathway enzyme is 1-deoxy-D-xylulose 5-phosphate synthase (DXS), 1-deoxy-D-xylulose 5-phosphate reductoisomerase (DXR); 2-C-methyl-D-erythritol 4-phosphate cytidylyltransferase (CMS); 4-diphosphocytidyl-2-C-methyl-D-erythritol kinase (CMK); 2-C-methyl-D-erythritol 2,4-cyclodiphosphate synthase (MECS); 4-hydroxy-3-methyl-but-2-enyl pyrophosphate synthase (HDS); 4-hydroxy-3-methyl-but-2-enyl pyrophosphate reductase (HDR); isopentenyl pyrophosphate isomerase (IDI), or geranyl pyrophosphate synthase (GPPS). In some embodiments, the MVA pathway enzyme is mevalonate 3-phosphate-5-kinase, isopentenyl-5-phosphate kinase, mevalonate-5-phosphate decarboxylase, or mevalonate-5-kinase. In some embodiments, the increased production of GPP results in increased production of the cannabinoids described herein, e.g., CBCA, CBDA, THCA, CBCOA, CBDOA, THCOA, CBCVA, CBDVA, THCVA, CBCO, CBDO, THCO, CBCV, CBDV, THCV, CBC, CBD, and/or THC, by the non-natural flavin-dependent oxidase described herein. In some embodiments, the increased production of GPP results in increased production of CBCA, THCA, CBCOA, CBCVA, CBCO, CBCV, and/or CBC, by the non-natural flavin-dependent oxidase described herein.

In some embodiments, the engineered cell produces GPP from an alternative non-MEP, non-MVA geranyl pyrophosphate pathway. In some embodiments, GPP is produced from a precursor selected from isoprenol, prenol, and geraniol. In some embodiments, the engineered cell expresses an exogenous or overexpresses an exogenous or endogenous gene that encodes any one of the enzymes in a non-MVA, non-MEP pathways, thereby increasing the production of GPP. In some embodiments, the non-MVA, non-MEP pathway enzyme is alcohol kinase, alcohol diphosphokinase, phosphate kinase, isopentenyl diphosphate isomerase, or geranyl pyrophosphate synthase (GPPS). In some embodiments, the increased production of GPP results in increased production of the cannabinoids described herein, e.g., CBCA, CBDA, THCA, CBCOA, CBDOA, THCOA, CBCVA, CBDVA, THCVA, CBCO, CBDO, THCO, CBCV, CBDV, THCV, CBC, CBD, and/or THC, by the non-natural flavin-dependent oxidase described herein. In some embodiments, the increased production of GPP results in increased production of CBCA by the non-natural flavin-dependent oxidase described herein.

In some embodiments, the engineered cell an exogenous or overexpresses an exogenous or endogenous GPP synthase. Non-limiting examples of GPP synthases include E. coli IspA (NP_414955), C. glutamicum IdsA (WP_11014931.1), and the enzymes listed in Table 2.

TABLE 2
Exemplary GPP Synthases

| | GenBank Accession | | GenBank Accession |
|---|---|---|---|
| Species | No. | Species | No. |
| Abies grandis | AAN01133.1 and | Corynebacterium | WP_035105251.1 |
| | AAN01134.1 | camporealensis | |
| Corynebacterium | WP_074025495.1 | Corynebacterium | WP_005328932.1 |
| crudilactis | | tuberculostearicum | |
| Corynebacterium | WP_096457048.1 | Corynebacterium | WP_005324491.1 |
| glutamicum | | pseudogenitalium | |
| Corynebacterium deserti | WP_053545301.1 | Corynebacterium | WP_083985528.1 |
| | | testudinoris | |
| Corynebacterium callunae | WP_015651699.1 | Corynebacterium stationis | WP_066793135.1 |
| Corynebacterium efficiens | WP_006768068.1 | Corynebacterium | WP_105324112.1 |
| | | sp. J010B-136 | |
| Corynebacterium | WP_080794061.1 | Corynebacterium | WP_123047545.1 |
| sp. Marseille-P2417 | | sp. CCUG 69366 | |
| Corynebacterium | WP_040086238.1 | Corynebacterium | WP_023030480.1 |
| humireducens | | sp. KPL1818 | |
| Corynebacterium | WP_015401326.1 | Corynebacterium accolens | WP_005283903.1 |
| halotolerans | | | |
| Corynebacterium | WP_042621772.1 | Corynebacterium | WP_126319428.1 |
| marinum | | segmentosum | |
| Corynebacterium | WP_042531577.1 | Corynebacterium | WP_121911356.1 |
| singulare | | macginleyi | |
| Corynebacterium | WP_115022907.1 | Pseudomonas aeruginosa | SQG59150.1 |
| minutissimum | | | |
| Corynebacterium | WP_143337494.1 | Streptococcus | VDG63248.1 |
| pollutisoli | | thermophilus | |
| Corynebacterium | WP_018297093.1 | Nocardia vermiculata | WP_084473733.1 |
| lubricantis | | | |
| Corynebacterium | WP_092284621.1 | Rhodococcus | WP_088945631.1 |
| spheniscorum | | sp. 1168 | |
| Corynebacterium | WP_018020857.1 | Clostridium | WP_113570111.1 |
| doosanense | | paraputrificum | |
| Corynebacterium | WP_075731219.1 | Nocardia cyriacigeorgica | WP_036535265.1 |
| flavescens | | | |
| Corynebacterium | WP_143334899.1 | Nocardia concava | WP_040806894.1 |
| aurimucosum | | | |
| Corynebacterium | WP_003845210.1 | Rhodococcus yunnanensis | WP_072806331.1 |
| ammoniagenes | | | |
| Corynebacterium | WP_086587718.1 | | |
| kefirresidentii | | | |

In some embodiments, the disclosure provides a composition comprising the non-natural flavin-dependent oxidase described herein and one or more of the OLS described herein, the OAC described herein, and the GPP pathway enzyme described herein. In some embodiments, the disclosure provides an engineered cell comprising the non-natural flavin-dependent oxidase described herein and one or more of the OLS described herein, the OAC described herein, and the GPP pathway enzyme described herein. In some embodiments, the disclosure provides one or more polynucleotides comprising one or more nucleic acid sequences encoding the non-natural flavin-dependent oxidase described herein and one or more of the OLS described herein, the OAC described herein, and the GPP pathway enzyme described herein. In some embodiments, the GPP pathway enzyme comprises geranyl pyrophosphate (GPP) synthase, famesyl pyrophosphate synthase, isoprenyl pyrophosphate synthase, geranylgeranyl pyrophosphate synthase, alcohol kinase, alcohol diphosphokinase, phosphate kinase, isopentenyl diphosphate isomerase, geranyl pyrophosphate synthase, or a combination thereof. In some embodiments, the disclosure provides an expression construct comprising the one or more polynucleotides. In some embodiments, the expression construct comprises a single expression vector. In some embodiments, the expression construct comprises more than one expression vector. In some embodiments, the disclosure provides an engineered cell comprising the one or more polynucleotides. In some embodiments, the disclosure provides an engineered cell comprising the expression construct. In some embodiments, the engineered cell produces CBCA, CBDA, THCA, CBCOA, CBDOA, THCOA, CBCVA, CBDVA, THCVA, CBCO, CBDO, THCO, CBCV, CBDV, THCV, CBC, CBD, and/or THC. In some embodiments, the engineered cell produces CBCA, CBCOA, CBCVA, CBCO, CBCV, and/or CBC. In some embodiments, the engineered cell produces CBCA.

### Prenyltransferase

In some embodiments, the engineered cell of the disclosure further comprises a prenyltransferase.

In general, the conversion of OA+GPP to CBGA (and the analogous conversions of OSA+GPP to CBGOA and DA+GPP to CBGVA) is performed by a prenyltransferase. In C. sativa, prenyltransferase is a transmembrane protein belonging to the UbiA superfamily of membrane proteins. Other prenyltransferases, e.g., aromatic prenyltransferases such as NphB from Streptomyces, which are non-transmembrane and soluble, can also catalyze conversion of OA to CBGA, OSA to CBGOA, and/or DA to CBGVA.

In some embodiments, the prenyltransferase is a natural prenyltransferase, e.g., wild-type prenyltransferase. In some embodiments, the prenyltransferase is a non-natural prenyltransferase. In some embodiments, the prenyltransferase comprises one or more amino acid substitutions relative to a wild-type prenyltransferase. In some embodiments, the one or more amino acid substitutions in the non-natural prenyltransferase increases the activity of the prenyltransferase as compared to a wild-type prenyltransferase.

In some embodiments, the prenyltransferase has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:8. In some embodiments, the prenyltransferase is a non-natural prenyltransferase comprising at least 1, at least 2, at least 3, at least 4, at least 5, at least 6, at least 7, at least 8, at least 9, or at least 10 amino acid variations at positions corresponding to SEQ ID NO:8.

Although the amino acid positions of prenyltransferase described herein are with reference to the corresponding amino acid sequence of SEQ ID NO:8, it is understood that the amino acid sequence of a non-natural prenyltransferase can include an amino acid variation at an equivalent position corresponding to a variant of SEQ ID NO:8. One of the skill in the art would understand that alignment methods can be used to align variations of SEQ ID NO:8 to identify the position in the prenyltransferase variant that corresponds to a position in SEQ ID NO:8. In some embodiments, SEQ ID NO:8 corresponds to the amino acid sequence of Streptomyces antibioticus AQJ23_4042 prenyltransferase.

In some embodiments, the prenyltransferase comprises an amino acid substitutions at position V45, F121, T124, Q159, M160, Y173, S212, V213, A230, T267, Y286, Q293, R294, L296, F300, or a combination thereof, wherein the position corresponds to SEQ ID NO:8. In some embodiments, the prenyltransferase comprises two or more amino acid substitutions at positions V45, F121, T124, Q159, M160, Y173, S212, V213, A230, T267, Y286, Q293, R294, L296, F300, or a combination thereof. In some embodiments, the prenyltransferase comprises two or more amino acid substitutions at positions V45, F121, T124, Q159, M160, Y173, S212, V213, A230, T267, Y286, Q293, R294, L296, F300, or a combination thereof. Prenyltransferase and non-natural variants thereof are further discussed, e.g., in WO2019/173770 and WO2021/046367.

In some embodiments, the amino acid substitution is selected from V45I, V45T, F121V, T124K, T124L, Q159S, M160L, M160S, Y173D, Y173K, Y173P, Y173Q, S212H, A230S, T267P, Y286V, Q293H, R294K, L296K, L296L, L296M, L296Q, F300Y, and a combination thereof.

In some embodiments, the prenyltransferase comprising an amino acid substitution as described herein is capable of a greater rate of formation of CBGA from GPP and OA, CBGOA from GPP and OSA, and/or CBGVA from GPP and DA as compared with wild-type prenyltransferase.

In some embodiments, the disclosure provides a composition comprising the non-natural flavin-dependent oxidase described herein and one or more of the OLS described herein, the OAC described herein, the GPP pathway enzyme described herein, and the prenyltransferase described herein. In some embodiments, the disclosure provides an engineered cell comprising the non-natural flavin-dependent oxidase described herein and one or more of the OLS described herein, the OAC described herein, the GPP pathway enzyme described herein, and the prenyltransferase described herein. In some embodiments, the disclosure provides one or more polynucleotides comprising one or more nucleic acid sequences encoding the non-natural flavin-dependent oxidase described herein and one or more of the OLS described herein, the OAC described herein, the GPP pathway enzyme described herein, and the prenyltransferase described herein. In some embodiments, the disclosure provides an expression construct comprising the one or more polynucleotides. In some embodiments, the expression construct comprises a single expression vector. In some embodiments, the expression construct comprises more than one expression vector. In some embodiments, the disclosure provides an engineered cell comprising the one or more polynucleotides. In some embodiments, the disclosure provides an engineered cell comprising the expression construct. In some embodiments, the engineered cell produces CBCA, CBDA, THCA, CBCOA, CBDOA, THCOA, CBCVA, CBDVA, THCVA, CBCO, CBDO, THCO, CBCV, CBDV, THCV, CBC, CBD, and/or THC. In some embodiments, the engineered cell produces CBCA, CBCOA, CBCVA, CBCO, CBCV, and/or CBC. In some embodiments, the engineered cell produces CBCA.

### Additional Strain Modifications

In some embodiments, the engineered cell of the disclosure further comprises a modification that facilitates the production of the cannabinoids described herein, e.g., CBCA, CBDA, THCA, CBCOA, CBDOA, THCOA, CBCVA, CBDVA, THCVA, CBCO, CBDO, THCO, CBCV, CBDV, THCV, CBC, CBD, and/or THC. In some embodiments, the modification increases production of a cannabinoid in the engineered cell compared with a cell not comprising the modification. In some embodiments, the modification increases efflux of a cannabinoid in the engineered cell compared with a cell not comprising the modification. In some embodiments, the cannabinoid is CBCA, CBCOA, CBCVA, CBCO, CBCV, and/or CBC. In some embodiments, the cannabinoid is CBCA. In some embodiments, the modification comprises expressing or upregulating the expression of an endogenous gene that facilitates production of a cannabinoid. In some embodiments, the modification comprises introducing and/or overexpression an exogenous and/or heterologous gene that facilitates production of a cannabinoid. In some embodiments, the modification comprises downregulating, disrupting, or deleting an endogenous gene that hinders production of a cannabinoid. Expression and/or overexpression of endogenous and exogenous genes, and downregulation, disruption and/or deletion of endogenous genes are described in embodiments herein.

In some embodiments, the engineered cell of the disclosure comprises one or more of the following modifications:
i) express one or more exogenous nucleic acid sequences or overexpress one or more endogenous genes encoding a protein having an ABC transporter permease activity;
ii) express one or more exogenous nucleic acid sequences or overexpress one or more endogenous genes encoding a protein having an ABC transporter ATP-binding protein activity;
iii) express one or more exogenous nucleic acids sequences or overexpress one or more endogenous genes selected from blc, ydhC, ydhG, or a homolog thereof;
iv) express one or more exogenous nucleic acids sequences or overexpress one or more endogenous genes selected from mlaD, mlaE, mlaF, or a homolog thereof;
v) express one or more exogenous nucleic acid sequences or overexpress one or more endogenous genes encoding a protein having a siderophore receptor protein activity or overexpress one or more endogenous genes encoding a protein having a siderophore receptor protein activity;
vi) comprise a disruption of or downregulation in the expression of a regulator of expression of one or more endogenous genes encoding a protein having an ABC transporter permease activity, a protein having an ABC transporter ATP-binding protein activity, a blc gene, a ybhG protein, a ydhC protein, a mlaD protein, mlaE protein, mlaF protein, or a protein having a siderophore receptor protein activity;
vii) express one or more exogenous nucleic acids sequences or overexpress one or more endogenous genes encoding a multi-domain protein having acetyl-CoA carboxylase activity (MD-ACC);
viii) express one or more exogenous nucleic acids sequences or overexpress one or more endogenous genes encoding acetyl-CoA carboxyltransferase subunit u, biotin carboxyl carrier protein, biotin carboxylase, or acetyl-CoA carboxyltransferase subunit R, or express one or more exogenous nucleic acids or overexpress one or more endogenous genes encoding acetyl-CoA carboxyltransferase, biotin carboxyl carrier protein, or biotin carboxylase activities;
ix) disruption of or downregulation in the expression of an endogenous gene encoding a protein having (acyl-carrier-protein) S-malonyltransferase activity, an endogenous gene encoding a protein having 3-hydroxypalmitoyl-(acyl-carrier-protein) dehydratase activity, or both;
x) express an exogenous nucleic acid sequence or overexpress an endogenous gene encoding a protein having fatty acyl-CoA ligase activity, or both;
xi) disruption of or downregulation in the expression of at least one endogenous gene encoding a protein having acyl-CoA dehydrogenase activity or enoyl-CoA hydratase activity;
xii) comprise a disruption of or downregulation in the expression of at least one endogenous gene encoding a protein having acyl-CoA esterase/thioesterase activity;
xiii) comprise a disruption of or downregulation in the expression of at least one endogenous gene encoding a repressor of transcription of one or more genes required for fatty acid beta-oxidation or an upregulator of fatty acid biosynthesis in combination with disruption or downregulation of one or more endogenous genes encoding one or more proteins of fatty acid beta-oxidation pathway; xiv) express an exogenous nucleic acid sequence or overexpress an endogenous gene encoding a protein having geranyl pyrophosphate synthase (GPPS), famesyl pyrophosphate synthase, isoprenyl pyrophosphate synthase, geranylgeranyl pyrophosphate synthase, alcohol kinase, alcohol diphosphokinase, phosphate kinase, isopentenyl diphosphate isomerase, geranyl pyrophosphate synthase, prenol kinase activity, prenol diphosphokinase activity, isoprenol kinase activity, isoprenol diphosphokinase activity, dimethylallyl phosphate kinase activity, isopentenyl phosphate kinase activity, or isopentenyl diphosphate isomerase activity;
xv) express an exogenous nucleic acid sequence or overexpress an endogenous gene encoding a protein having GPP synthase activity;
xvi) express an exogenous nucleic acid sequence encoding an olivetol synthase;
xvii) express an exogenous nucleic acid sequence encoding an olivetolic acid cyclase;
xviii) express an exogenous nucleic acid sequence encoding a prenyltransferase;
xix) express one or more exogenous nucleic acid sequences or overexpressing one or more endogenous genes encoding one or more enzymes of MVA pathway, MEP pathway, or a non-MVA, non-MEP pathway;
xx) express an exogenous nucleic acid sequence or overexpress an endogenous gene encoding a biotin-(acetyl-CoA carboxylase) ligase;
xxi) express an exogenous nucleic acid sequence or overexpress an endogenous gene encoding a isopentenyl-diphosphate delta-isomerase;
xxii) express an exogenous nucleic acid sequence or overexpress an endogenous gene encoding a hydroxyethylthiazole kinase or both;
xxiii) express an exogenous nucleic acid sequence or overexpress an endogenous gene encoding a Type III pantothenate kinase; and
xxiv) comprise a disruption of or downregulation in the expression of at least one endogenous gene encoding a phosphatase selected from the group consisting of ADP-sugar pyrophosphatase, dihydroneopterin triphosphate diphosphatase, pyrimidine deoxynucleotide diphosphatase, pyrimidine pyrophosphate phosphatase, and Nudix hydrolase.

In some embodiments, the disclosure provides an engineered cell comprising the non-natural flavin-dependent oxidase described herein and one or more of the OLS described herein, the OAC described herein, the GPP pathway enzyme described herein, the prenyltransferase described herein, and an additional modification described herein. In some embodiments, the disclosure provides one or more polynucleotides comprising one or more nucleic acid sequences encoding the non-natural flavin-dependent oxidase described herein and one or more of the OLS described herein, the OAC described herein, the GPP pathway enzyme described herein, the prenyltransferase described herein, and an additional modification described herein. In some embodiments, the disclosure provides an expression construct comprising the one or more polynucleotides. In some embodiments, the expression construct comprises a single expression vector. In some embodiments, the expression construct comprises more than one expression vector. In some embodiments, the disclosure provides an engineered cell comprising the one or more polynucleotides. In some embodiments, the disclosure provides an engineered cell comprising the expression construct. In some embodiments, the engineered cell produces CBCA, CBDA, THCA, CBCOA, CBDOA, THCOA, CBCVA, CBDVA, THCVA, CBCO, CBDO, THCO, CBCV, CBDV, THCV, CBC, CBD, and/or THC. In some embodiments, the engineered cell produces CBCA, CBCOA, CBCVA, CBCO, CBCV, and/or CBC. In some embodiments, the engineered cell produces CBCA.

### Host Cells

A variety of microorganisms may be suitable as the engineered cell described herein. Such organisms include both prokaryotic and eukaryotic organisms including, but not limited to, bacteria, including archaea and eubacteria, and eukaryotes, including yeast, plant, and insect. Nonlimiting examples of suitable microbial hosts for the bio-production of a cannabinoid include, but are not limited to, any Gram negative organisms, more particularly a member of the family Enterobacteriaceae, such as E. coli, or Oligotropha carboxidovorans, or a Pseudomononas sp.; any Gram positive microorganism, for example Bacillus subtilis, Lactobaccilus sp. or Lactococcus sp.; a yeast, for example Saccharomyces cerevisiae, Pichia pastoris or Pichia stipitis; and other groups or microbial species. In some embodiments, the microbial host is a member of the genera Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Pichia, Candida, Hansenula, or Saccharomyces. In some embodiments, the microbial host is Oligotropha carboxidovorans (such as strain OM5), Escherichia coli, Alcaligenes eutrophus (Cupriavidus necator), Bacillus licheniformis, Paenibacillus macerans, Rhodococcus erythropolis, Pseudomonas putida, Lactobacillus plantarum, Enterococcus faecium, Enterococcus gallinarium, Enterococcus faecalis, Bacillus subtilis or Saccharomyces cerevisiae.

Further exemplary species are reported in U.S. Pat. No. 9,657,316 and include, for example, Escherichia coli, Saccharomyces cerevisiae, Saccharomyces kluyveri, Candida boidinii, Clostridium kluyveri, Clostridium acetobutylicum, Clostridium beijerinckii, Clostridium saccharoperbutylacetonicum, Clostridium perfringens, Clostridium difficile, Clostridium botulinum, Clostridium tyrobutyricum, Clostridium tetanomorphum, Clostridium tetani, Clostridium propionicum, Clostridium aminobutyricum, Clostridium subterminale, Clostridium sticklandii, Ralstonia eutropha, Mycobacterium bovis, Mycobacterium tuberculosis, Porphyromonas gingivalis, Arabidopsis thaliana, Thermus thermophilus, Pseudomonas species, including Pseudomonas aeruginosa, Pseudomonas putida, Pseudomonas stutzeri, Pseudomonas fluorescens, Homo sapiens, Oryctolagus cuniculus, Rhodobacter spaeroides, Thermoanaerobacter brockii, Metallosphaera sedula, Leuconostoc mesenteroides, Chloroflexus aurantiacus, Roseiflexus castenholzii, Erythrobacter, Simmondsia chinensis, Acinetobacter species, including Acinetobacter calcoaceticus and Acinetobacter baylyi, Porphyromonas gingivalis, Sulfolobus tokodaii, Sulfolobus solfataricus, Sulfolobus acidocaldarius, Bacillus subtilis, Bacillus cereus, Bacillus megaterium, Bacillus brevis, Bacillus pumilus, Rattus norvegicus, Klebsiella pneumonia, Klebsiella oxytoca, Euglena gracilis, Treponema denticola, Moorella thermoacetica, Thermotoga maritima, Halobacterium salinarum, Geobacillus stearothermophilus, Aeropyrum pernix, Sus scrofa, Caenorhabditis elegans, Corynebacterium glutamicum, Acidaminococcus fermentans, Lactococcus lactis, Lactobacillus plantarum, Streptococcus thermophilus, Enterobacter aerogenes, Candida, Aspergillus terreus, Pedicoccus pentosaceus, Zymomonas mobilus, Acetobacter pasteurians, Kluyveromyces lactis, Eubacterium barkeri, Bacteroides capillosus, Anaerotruncus colihominis, Natranaerobius thermophilusm, Campylobacter jejuni, Haemophilus influenzae, Serratia marcescens, Citrobacter amalonaticus, Myxococcus xanthus, Fusobacterium nuleatum, Penicillium chrysogenum, marine gamma proteobacterium, butyrate-producing bacterium, Nocardia iowensis, Nocardia farcinica, Streptomyces griseus, Schizosaccharomyces pombe, Geobacillus thermoglucosidasius, Salmonella typhimurium, Vibrio cholera, Heliobacter pylori, Nicotiana tabacum, Oryza sativa, Haloferax mediterranei, Agrobacterium tumefaciens, Achromobacter denitrificans, Fusobacterium nucleatum, Streptomyces clavuligenus, Acinetobacter baumanii, Mus musculus, Lachancea kluyveri, Trichomonas vaginalis, Trypanosoma brucei, Pseudomonas stutzeri, Bradyrhizobium japonicum, Mesorhizobium loti, Bos taurus, Nicotiana glutinosa, Vibrio vulnificus, Selenomonas ruminantium, Vibrio parahaemolyticus, Archaeoglobus fulgidus, Haloarcula marismortui, Pyrobaculum aerophilum, Mycobacterium smegmatis MC2 155, Mycobacterium avium subsp. paratuberculosis K-10, Mycobacterium marinum M, Tsukamurella paurometabola DSM 20162, Cyanobium PCC7001, Dictyostelium discoideum AX4, as well as other exemplary species disclosed herein or available as source organisms for corresponding genes.

In some embodiments, the engineered cell is a bacterial cell or a fungal cell. In some embodiments, the engineered cell is a bacterial cell. In some embodiments, the engineered cell is a yeast cell. In some embodiments, the engineered cell is an algal cell. In some embodiments, the engineered cell is a cyanobacterial cell. In some embodiments, the bacteria is Escherichia, Corynebacterium, Bacillus, Ralstonia, Zymomonas, or Staphylococcus. In some embodiments, the bacterial cell is an Escherichia coli cell.

In some embodiments, the engineered cell is an organism selected from Acinetobacter baumannii Naval-82, Acinetobacter sp. ADP1, Acinetobacter sp. strain M-1, Actinobacillus succinogenes 130Z, Allochromatium vinosum DSM 180, Amycolatopsis methanolica, Arabidopsis thaliana, Atopobium parvulum DSM 20469, Azotobacter vinelandii DJ, Bacillus alcalophilus ATCC 27647, Bacillus azotoformans LMG 9581, Bacillus coagulans 36D1, Bacillus megaterium, Bacillus methanolicus MGA3, Bacillus methanolicus PB1, Bacillus selenitireducens MLS10, Bacillus smithii, Bacillus subtilis, Burkholderia cenocepacia, Burkholderia cepacia, Burkholderia multivorans, Burkholderia pyrrocinia, Burkholderia stabilis, Burkholderia thailandensis E264, Burkholderiales bacterium Joshi_001, Butyrate-producing bacterium L2-50, Campylobacter jejuni, Candida albicans, Candida boidinii, Candida methylica, Carboxydothermus hydrogenoformans, Carboxydothermus hydrogenoformans Z-2901, Caulobacter sp. AP07, Chloroflexus aggregans DSM 9485, Chloroflexus aurantiacus J-10-fl, Citrobacter freundii, Citrobacter koseri ATCC BAA-895, Citrobacter youngae, Clostridium, Clostridium acetobutylicum, Clostridium acetobutylicum ATCC 824, Clostridium acidurici, Clostridium aminobutyricum, Clostridium asparagiforme DSM 15981, Clostridium beijerinckii, Clostridium beijerinckii NCIMB 8052, Clostridium bolteae ATCC BAA-613, Clostridium carboxidivorans P7, Clostridium cellulovorans 743B, Clostridium difficile, Clostridium hiranonis DSM 13275, Clostridium hylemonae DSM 15053, Clostridium kluyveri, Clostridium kluyveri DSM 555, Clostridium ljungdahli, Clostridium ljungdahlii DSM 13528, Clostridium methylpentosum DSM 5476, Clostridium pasteurianum, Clostridium pasteurianum DSM 525, Clostridium perfringens, Clostridium perfringens ATCC 13124, Clostridium perfringens str. 13, Clostridium phytofermentans ISDg, Clostridium saccharobutylicum, Clostridium saccharoperbutylacetonicum, Clostridium saccharoperbutylacetonicum N1-4, Clostridium tetani, Corynebacterium glutamicum ATCC 14067, Corynebacterium glutamicum R, Corynebacterium sp. U-96, Corynebacterium variabile, Cupriavidus necator N-1, Cyanobium PCC7001, Desulfatibacillum alkenivorans AK-01, Desulfitobacterium hafniense, Desulfitobacterium metallireducens DSM 15288, Desulfotomaculum reducens MI-1, Desulfovibrio africanus str. Walvis Bay, Desulfovibrio fructosovorans JJ, Desulfovibrio vulgaris str. Hildenborough, Desulfovibrio vulgaris str. ‘Miyazaki F’, Dictyostelium discoideum AX4, Escherichia coli, Escherichia coli K-12, Escherichia coli K-12 MG1655, Eubacterium hallii DSM 3353, Flavobacterium frigoris, Fusobacterium nucleatum subsp. polymorphum ATCC 10953, Geobacillus sp. Y4.IMC1, Geobacillus themodenitrificans NG80-2, Geobacter bemidjiensis Bem, Geobacter sulfurreducens, Geobacter sulfurreducens PCA, Geobacillus stearothermophilus DSM 2334, Haemophilus influenzae, Helicobacter pylori, Homo sapiens, Hydrogenobacter thermophilus, Hydrogenobacter thermophilus TK-6, Hyphomicrobium denitrificans ATCC 51888, Hyphomicrobium zavarzinii, Klebsiella pneumoniae, Klebsiella pneumoniae subsp. pneumoniae MGH 78578, Lactobacillus brevis ATCC 367, Leuconostoc mesenteroides, Lysinibacillus fusiformis, Lysinibacillus sphaericus, Mesorhizobium loti MAFF303099, Metallosphaera sedula, Methanosarcina acetivorans, Methanosarcina acetivorans C₂A, Methanosarcina barkeri, Methanosarcina mazei Tuc01, Methylobacter marinus, Methylobacterium extorquens, Methylobacterium extorquens AM1, Methylococcus capsulatas, Methylomonas aminofaciens, Moorella thermoacetica, Mycobacter sp. strain JC1 DSM 3803, Mycobacterium avium subsp. paratuberculosis K-10, Mycobacterium bovis BCG, Mycobacterium gastri, Mycobacterium marinum M, Mycobacterium smegmatis, Mycobacterium smegmatis MC2 155, Mycobacterium tuberculosis, Nitrosopumilus salaria BD31, Nitrososphaera gargensis Ga9.2, Nocardia farcinica IFM 10152, Nocardia iowensis (sp. NRRL 5646), Nostoc sp. PCC 7120, Ogataea angusta, Ogataea parapolymorpha DL-1 (Hansenula polymorpha DL-1), Paenibacillus peoriae KCTC 3763, Paracoccus denitrificans, Penicillium chrysogenum, Photobacterium profundum 3TCK, Phytofermentans ISDg, Pichia pastoris, Picrophilus torridus DSM9790, Porphyromonas gingivalis, Porphyromonas gingivalis W83, Pseudomonas aeruginosa PA01, Pseudomonas denitrificans, Pseudomonas knackmussii, Pseudomonas putida, Pseudomonas sp, Pseudomonas syringae pv. syringae B728a, Pyrobaculum islandicum DSM 4184, Pyrococcus abyssi, Pyrococcus furiosus, Pyrococcus horikoshii OT3, Ralstonia eutropha, Ralstonia eutropha H16, Rhodobacter capsulatus, Rhodobacter sphaeroides, Rhodobacter sphaeroides ATCC 17025, Rhodopseudomonas palustris, Rhodopseudomonas palustris CGA009, Rhodopseudomonas palustris DX-1, Rhodospirillum rubrum, Rhodospirillum rubrum ATCC 11170, Ruminococcus obeum ATCC 29174, Saccharomyces cerevisiae, Saccharomyces cerevisiae S288c, Salmonella enterica, Salmonella enterica subsp. enterica serovar Typhimurium str. LT2, Salmonella enterica typhimurium, Salmonella typhimurium, Schizosaccharomyces pombe, Sebaldella termitidis ATCC 33386, Shewanella oneidensis MR-1, Sinorhizobium meliloti 1021, Streptomyces coelicolor, Streptomyces griseus subsp. griseus NBRC 13350, Sulfolobus acidocalarius, Sulfolobus solfataricus P-2, Synechocystis str. PCC 6803, Syntrophobacter fumaroxidans, Thauera aromatica, Thermoanaerobacter sp. X514, Thermococcus kodakaraensis, Thermococcus litoralis, Thermoplasma acidophilum, Thermoproteus neutrophilus, Thermotoga maritima, Thiocapsa roseopersicina, Tolumonas auensis DSM 9187, Trichomonas vaginalis G3, Trypanosoma brucei, Tsukamurella paurometabola DSM 20162, Vibrio cholera, Vibrio harveyi ATCC BAA-1116, Xanthobacter autotrophicus Py2, Yersinia intermedia, and Zea mays.

Algae that can be engineered for cannabinoid production include, but are not limited to, unicellular and multicellular algae. Examples of such algae can include a species of rhodophyte, chlorophyte, heterokontophyte (including diatoms), tribophyte, glaucophyte, chlorarachniophyte, euglenoid, haptophyte, cryptomonad, dinoflagellum, phytoplankton, and the like, and combinations thereof. In one embodiment, algae can be of the classes Chlorophyceae and/or Haptophyta.

Microalgae (single-celled algae) produce natural oils that can contain the synthesized cannabinoids. Specific species that are considered for cannabinoid production include, but are not limited to, Neochloris oleoabundans, Scenedesmus dimorphus, Euglena gracilis, Phaeodactylum tricornutum, Pleurochrysis carterae, Prymnesium parvum, Tetraselmis chui, Nannochloropsis gaditiana. Dunaliella salina. Dunaliella tertiolecta, Chlorella vulgaris, Chlorella variabilis, and Chlamydomonas reinhardtii. Additional or alternate algal sources can include one or more microalgae of the Achnanthes, Amphiprora, Amphora, Ankistrodesmus, Asteromonas, Boekelovia, Borodinella, Botryococcus, Bracteococcus, Chaetoceros, Carteria, Chlamydomonas, Chlorococcum, Chlorogonium, Chlorella, Chroomonas, Chrsosphaera, Cricosphaera, Crypthecodinium, Cryptomonas, Cyclotella, Dunaliella, Ellipsoidon, Emiliania. Eremosphaera, Ernodesmius, Euglena, Franceia, Fragilaria, Gloeolhamnion, Haematococcus, Halocafeteria, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Phaeodactylum, Phagus. Platymonas, Pleurochrsis, Pleurococcus, Prototheca, Pseudochlorella, Pyramimonas, Pvrobotrys, Scenedesmus, Skeletonema, Spyrogyra, Stichococcus, Tetraselmis, Thalassiosira, Viridiella, and Volvox species, and/or one or more cyanobacteria of the Agmenellum, Anabaena, Anabaenopsis, Anacystis, Aphanizomenon, Arthrospira, Asterocapsa, Borzia, Calothrix, Chamaesiphon, Chlorogloeopsis, Chroococcidiopsis, Chroococcus, Crinalium, Cyanobacterium, Cyanobium, Cyanocystis, Cyanospira, Cyanothece, Cylindrospermopsis, Cylindrospermum, Dactylcoccopsis, Dermocarpella, Fischerella, Fremyella, Geitleria, Geitlerinema, Gloeobacter, Gloeocapsa, Gloeothece, Halospirulina, Ivengariella, Leptolyngbya, Limnothrix, Lyngbya, Microcoleus, Microcystis, Mxosarcina, Nodularia, Nostoc, Nostochopsis, Oscillatoria, Phormidium, Planktothrix, Pleurocapsa, Prochlorococcus, Prochloron, Prochlorothrix, Pseudanabaena, Rivularia, Schizothrix, Scvtonema, Spirulina, Stanieria, Starria, Stigonema, Symploca, Synechococcus, Svnechocystis, Tolipothrix, Trichodesmium. Tychonema, and Xenococcus species.

The host cell may be genetically modified for a recombinant production system, e.g., to produce CBCA, CBDA, THCA, CBCOA, CBDOA, THCOA, CBCVA, CBDVA, THCVA, CBCO, CBDO, THCO, CBCV, CBDV, THCV, CBC, CBD, and/or THC as described herein. In some embodiments, the host cell is genetically modified to produce CBCA, CBCOA, CBCVA, CBCO, CBCV, and/or CBC as described herein. In some embodiments, the host cell is genetically modified to produce CBCA. The mode of gene transfer technology may be by electroporation, conjugation, transduction or natural transformation as described herein.

To genetically modify a host cell of the disclosure, one or more heterologous nucleic acids disclosed herein is introduced stably or transiently into a host cell, using established techniques. Such techniques may include, but are not limited to, electroporation, calcium phosphate precipitation, DEAE-dextran mediated transfection, liposome-mediated transfection, particle bombardment, and the like. For stable transformation, a heterologous nucleic acid will generally further include a selectable marker, e.g., any of several well-known selectable markers such as neomycin resistance, ampicillin resistance, tetracycline resistance, chloramphenicol resistance, kanamycin resistance, hygromycin resistance, G418 resistance, bleomycin resistance, zeocin resistance, and the like. A broad range of plasmids and drug resistance markers are available and described in embodiments herein. The cloning vectors are tailored to the host organisms based on the nature of antibiotic resistance markers that can function in that host cell. In some embodiments, the host cell is genetically modified using CRISPR/Cas9 to produce the engineered cell of the disclosure.

### Fermentation

In some embodiments, the disclosure provides a method of producing a cannabinoid or precursor thereof, e.g., CBCA, CBDA, THCA, CBCOA, CBDOA, THCOA, CBCVA, CBDVA, THCVA, CBCO, CBDO, THCO, CBCV, CBDV, THCV, CBC, CBD, and/or THC, as described herein, comprising culturing an engineered cell provided herein to provide the cannabinoid. In some embodiments, the method further comprises recovering the cannabinoid, e.g., CBCA, CBDA, THCA, CBCOA, CBDOA, THCOA, CBCVA, CBDVA, THCVA, CBCO, CBDO, THCO, CBCV, CBDV, THCV, CBC, CBD, and/or THC from the cell, cell extract, culture medium, whole culture, or a combination thereof. In some embodiments, the cannabinoid is CBCA, CBCOA, CBCVA, CBCO, CBCV, CBC, or a combination thereof. In some embodiments, the cannabinoid is CBCA.

In some embodiments, the culture medium of the engineered cells further comprises at least one carbon source. In embodiments where the cells are heterotrophic cells, the culture medium comprises at least one carbon source that is also an energy source, also known as a “feed molecule.” In some embodiments, the culture medium comprises one, two, three, or more carbon sources that are not primary energy sources. Non-limiting examples of feed molecules that can be included in the culture medium include acetate, malonate, oxaloacetate, aspartate, glutamate, beta-alanine, alpha-alanine, butyrate, hexanoate, hexanol, prenol, isoprenol, and geraniol. Further examples of compounds that can be provided in the culture medium include, without limitation, biotin, thiamine, pantotheine, and 4-phosphopantetheine.

In some embodiments, the culture medium comprises acetate. In some embodiments, the culture medium comprises acetate and hexanoate. In some embodiments, the culture medium comprises malonate and hexanoate. In some embodiments, the culture medium comprises prenol, isoprenol, and/or geraniol. In some embodiments, the culture medium comprises aspartate, hexanoate, prenol, isoprenol, and/or geraniol.

Depending on the desired microorganism or strain to be used, the appropriate culture medium may be used. For example, descriptions of various culture media may be found in “Manual of Methods for General Bacteriology” of the American Society for Bacteriology (Washington D.C., USA, 1981). As used herein, culture medium, or simply “medium” as it relates to the growth source, refers to the starting medium, which may be in a solid or liquid form. “Cultured medium” as used herein refers to medium (e.g. liquid medium) containing microbes that have been fermentatively grown and can include other cellular biomass. The medium generally includes one or more carbon sources, nitrogen sources, inorganic salts, vitamins and/or trace elements. “Whole culture” as used herein refers to cultured cells plus the culture medium in which they are cultured. “Cell extract” as used herein refers to a lysate of the cultured cells, which may include the culture medium and which may be crude (unpurified), purified or partially purified. Methods of purifying cell lysates are known to the skilled artisan and described in embodiments herein.

Exemplary carbon sources include sugar carbons such as sucrose, glucose, galactose, fructose, mannose, isomaltose, xylose, maltose, arabinose, cellobiose and 3-, 4-, or 5-oligomers thereof. Other carbon sources include carbon sources such as methanol, ethanol, glycerol, formate and fatty acids. Still other carbon sources include carbon sources from gas such as synthesis gas, waste gas, methane, CO, CO₂ and any mixture of CO, CO₂ with H₂. Other carbon sources can include renewal feedstocks and biomass. Exemplary renewal feedstocks include cellulosic biomass, hemicellulosic biomass and lignin feedstocks.

In some embodiments, the engineered cell is sustained, cultured, or fermented under aerobic, microaerobic, anaerobic or substantially anaerobic conditions. Exemplary aerobic, microaerobic, and anaerobic conditions have been described previously and are known in the art. Briefly, anaerobic conditions refer to an environment devoid of oxygen. Substantially anaerobic conditions include, for example, a culture, batch fermentation or continuous fermentation such that the dissolved oxygen concentration in the medium remains between 0 and 10% of saturation, or higher. Substantially anaerobic conditions also include growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases. Exemplary anaerobic conditions for fermentation processes are described, for example, in US2009/0047719. Any of these conditions can be employed with the microbial organisms described herein as well as other anaerobic conditions known in the field. The culture conditions can include, for example, liquid culture procedures as well as fermentation and other large scale culture procedures.

The culture conditions can be scaled up and grown continuously for manufacturing the cannabinoid products described herein. Exemplary growth procedures include, for example, fed-batch fermentation and batch separation; fed-batch fermentation and continuous separation, or continuous fermentation and continuous separation. Fermentation procedures can be particularly useful for the biosynthetic production of commercial quantities of cannabinoids, e.g., CBCA, CBDA, THCA, CBCOA, CBDOA, CBCVA, CBDVA, THCVA, THCOA, CBCO, CBDO, THCO, CBCV, CBDV, THCV, CBC, CBD, and/or THC. In some embodiments, the cannabinoid is CBCA, CBCOA, CBCVA, CBCO, CBCV, CBC, or a combination thereof. In some embodiments, the cannabinoid is CBCA. Generally, and as with non-continuous culture procedures, the continuous and/or near-continuous production of cannabinoid product can include culturing a cannabinoid-producing organism with sufficient nutrients and medium to sustain and/or nearly sustain growth in an exponential phase. Continuous culture under such conditions can include, for example, 1 day, 2, 3, 4, 5, 6 or 7 days or more. Additionally, continuous culture can include 1 week, 2, 3, 4 or 5 or more weeks and up to several months. Alternatively, the desired microorganism can be cultured for hours, if suitable for a particular application. It is to be understood that the continuous and/or near-continuous culture conditions also can include all time intervals in between these exemplary periods. It is further understood that the time of culturing the microbial organism is for a sufficient period of time to produce a sufficient amount of product for a desired purpose.

Fermentation procedures are known to the skilled artisan. Briefly, fermentation for the biosynthetic production of a cannabinoid, e.g., CBCA, CBDA, THCA, CBCOA, CBDOA, THCOA, CBCVA, CBDVA, THCVA, CBCO, CBDO, THCO, CBCV, CBDV, THCV, CBC, CBD, and/or THC, can be utilized in, for example, fed-batch fermentation and batch separation; fed-batch fermentation and continuous separation, or continuous fermentation and continuous separation. Examples of batch and continuous fermentation procedures are known in the field. Typically, cells are grown at a temperature in the range of about 25° C. to about 40° C. in an appropriate medium, as well as up to 70° C. for thermophilic microorganisms. In some embodiments, the cannabinoid is CBCA, CBCOA, CBCVA, CBCO, CBCV, CBC, or a combination thereof. In some embodiments, the cannabinoid is CBCA.

The culture medium at the start of fermentation may have a pH of about 4 to about 7. The pH may be less than 11, less than 10, less than 9, or less than 8. In some embodiments, the pH is at least 2, at least 3, at least 4, at least 5, at least 6, or at least 7. In some embodiments, the pH of the medium is about 6 to about 9.5; 6 to about 9, about 6 to 8 or about 8 to 9.

In some embodiments, upon completion of the cultivation period, the fermenter contents are passed through a cell separation unit, for example, a centrifuge, filtration unit, and the like, to remove cells and cell debris. In embodiments where the desired product is expressed intracellularly, the cells are lysed or disrupted enzymatically or chemically prior to or after separation of cells from the fermentation broth, as desired, in order to release additional product. The fermentation broth can be transferred to a product separations unit. Isolation of product can be performed by standard separations procedures employed in the art to separate a desired product from dilute aqueous solutions. Such methods include, but are not limited to, liquid-liquid extraction using a water immiscible organic solvent (e.g., toluene or other suitable solvents, including but not limited to diethyl ether, ethyl acetate, methylene chloride, chloroform, benzene, pentane, hexane, heptane, petroleum ether, methyl tertiary butyl ether (MTBE), and the like) to provide an organic solution of the product, if appropriate, standard distillation methods, and the like, depending on the chemical characteristics of the product of the fermentation process.

Suitable purification and/or assays to test a cannabinoid, e.g., CBCA, CBDA, THCA, CBCOA, CBDOA, THCOA, CBCVA, CBDVA, THCVA, CBCO, CBDO, THCO, CBCV, CBDV, THCV, CBC, CBD, and/or THC, produced by the methods herein can be performed using known methods. In some embodiments, the cannabinoid is CBCA, CBCOA, CBCVA, CBCO, CBCV, CBC, or a combination thereof. In some embodiments, the cannabinoid is CBCA. For example, product and byproduct formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al. (2005), Biotechnol. Bioeng. 90:775-779), or other suitable assay and detection methods well known in the art. The individual enzyme or protein activities from the exogenous DNA sequences can also be assayed using methods known in the art.

The cannabinoids produced using methods described herein can be separated from other components in the culture using a variety of methods well known in the art. Such separation methods include, for example, extraction procedures as well as methods that include liquid-liquid extraction, pervaporation, evaporation, filtration, membrane filtration (including reverse osmosis, nanofiltration, ultrafiltration, and microfiltration), membrane filtration with diafiltration, membrane separation, reverse osmosis, electrodialysis, distillation, extractive distillation, reactive distillation, azeotropic distillation, crystallization and recrystallization, centrifugation, extractive filtration, ion exchange chromatography, size exclusion chromatography, adsorption chromatography, carbon adsorption, hydrogenation, and ultrafiltration. For example, the amount of cannabinoid or other product(s), including a polyketide, produced in a bio-production media generally can be determined using any of methods such as, for example, high performance liquid chromatography (HPLC), gas chromatography (GC), GC/Mass Spectroscopy (MS), or spectrometry.

In some embodiments, the cell extract or cell culture medium described herein comprises a cannabinoid. In some embodiments, the cannabinoid is cannabichromene (CBC) type (e.g. cannabichromenic acid), cannabigerol (CBG) type (e.g. cannabigerolic acid), cannabidiol (CBD) type (e.g. cannabidiolic acid), Δ⁹-trans-tetrahydrocannabinol (Δ⁹-THC) type (e.g. Δ⁹-tetrahydrocannabinolic acid), Δ⁸-trans-tetrahydrocannabinol (Δ⁸-THC) type, cannabicyclol (CBL) type, cannabielsoin (CBE) type, cannabinol (CBN) type, cannabinodiol (CBND) type, cannabitriol type, or a combination thereof. In some embodiments, the cannabinoid is cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C4 (CBD-C4), cannabidivarinic acid (CBDVA), cannabidivarin (CBDV), cannabidiorcol (CBD-C1), Δ⁹-tetrahydrocannabinolic acid A (THCA-A), Δ⁹-tetrahydrocannabinolic acid B (THCA-B), Δ⁹-tetrahydrocannabinol (THC), Δ⁹-tetrahydrocannabinolic acid-C4 (THCA-C4), Δ⁹-tetrahydrocannabinol-C4 (THC-C4), Δ⁹-tetrahydrocannabivarinic acid (THCVA), Δ⁹-tetrahydrocannabivarin (THCV), Δ⁹-tetrahydrocannabiorcolic acid (THCA-C1), Δ⁹-tetrahydrocannabiorcol (THC-C1), Δ⁷-cis-iso-tetrahydrocannabivarin, Δ⁸-tetrahydrocannabinolic acid (Δ⁸-THCA), Δ⁸-tetrahydrocannabinol (Δ⁸-THC), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabielsoinic acid, cannabicitranic acid, cannabinolic acid (CBNA), cannabinol (CBN), cannabinol methylether (CBNM), cannabinol-C4, (CBN-C4), cannabivarin (CBV), cannabinol-C2 (CNB-C2), cannabiorcol (CBN-C1), cannabinodiol (CBND), cannabinodivarin (CBVD), cannabitriol, 10-ethyoxy-9-hydroxy-delta-6a-tetrahydrocannabinol, 8,9-dihydroxyl-delta-6a-tetrahydrocannabinol, cannabitriolvarin (CBTVE), dehydrocannabifuran (DCBF), cannabifuran (CBF), cannabichromanon (CBCN), cannabicitran, 10-oxo-delta-6a-tetrahydrocannabinol (OTHC), Δ⁹-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR), trihydroxy-Δ⁹-tetrahydrocannabinol (triOH-THC), or a combination thereof.

In some embodiments, the disclosure provides a cell extract or cell culture medium comprising cannabigerolic acid (CBGA), cannabichromenic acid (CBCA), cannabidiolic acid (CBDA), tetrahydrocannabinolic acid (THCA), cannabigerol (CBG), cannabichromene (CBC), cannabidiol (CBD), tetrahydrocannabinol (THC), cannabigerorcinic acid (CBGOA), cannabiorcichromenic acid (CBCOA), cannabidiorcinic acid (CBDOA), tetraydrocannabiorcolic acid (THCOA), cannabigerivarinic acid (CBGVA), cannabichromevarinic acid (CBCVA), cannabidivarinic acid (CBDVA), tetrahydrocannabivarin acid (THCVA), cannabigerorcinol (CBGO), cannabichromeorcin (CBCO), cannabidiorcin (CBDO), tetrahydrocannabiorcin (THCO), cannabigerivarinol (CBGV), cannabichromevarin (CBCV), cannabidivarin (CBDV), tetrahydrocannabivarin (THCV), an isomer, analog or derivative thereof, or a combination thereof derived from the engineered cell described herein. In some embodiments, the disclosure provides a cell extract or cell culture medium comprising CBGA, CBCA, CBG, CBC, CBGOA, CBCOA, CBGVA, CBCVA, CBGO, CBCO, CBGO, CBCV, an isomer, analog or derivative thereof, or a combination thereof derived from the engineered cell described herein. In some embodiments, the cell extract or cell culture medium comprises CBGA and CBCA. In some embodiments, a derivative of a cannabinoid described herein, e.g., CBGA, CBCA, CBDA, THCA, CBGOA, CBCOA, CBDOA, THCOA, CBGVA, CBCVA, CBDVA, and/or THCVA, is a decarboxylated form of the cannabinoid.

### Method of Making or Isolating

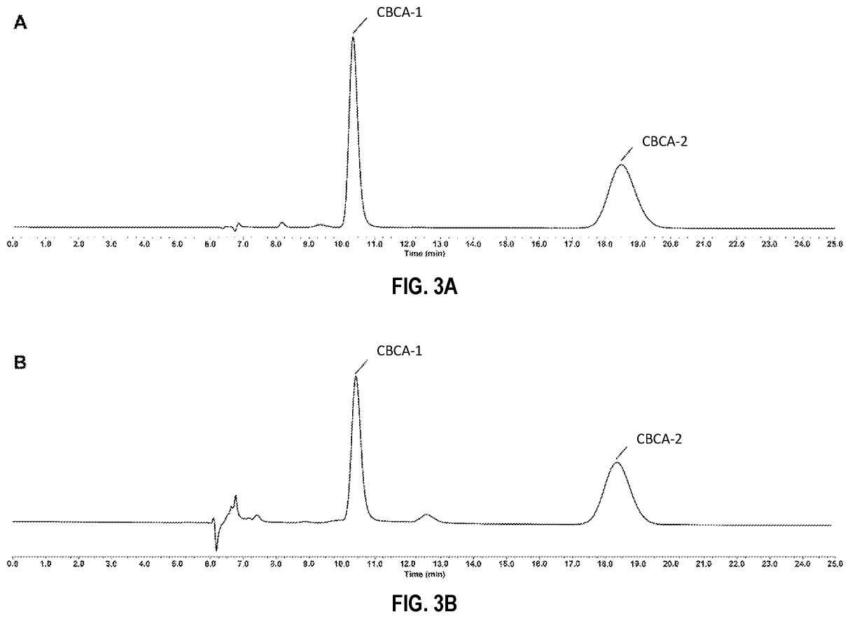

In some embodiments, the disclosure provides a method of making a cannabinoid selected from CBCA, CBC, CBCOA, CBCVA, CBCO, CBCV, CBDA, CBD, CBDOA, CBDVA, CBCO, CBDV, THCA, THC, THCOA, THCVA, THCO, THCV, an isomer, analog or derivative thereof, or a combination thereof, comprising culturing the engineered cell as described herein, or isolating the cannabinoid from the cell extract or cell culture medium as described herein. In some embodiments, the cannabinoid is CBCA, CBC, CBCOA, CBCVA, CBCO, CBCV, an isomer, analog or derivative thereof, or a combination thereof. In some embodiments, the cannabinoid is CBCA. In some embodiments, the method makes one or both regioisomers of CBCA, e.g., CBCA-A and CBCA-B. Each of CBCA-A and CBCA-B comprises two enantiomers, (R)-CBCA and (S)-CBCA. The enantiomers of CBCA-A are shown in FIG. 2. The enantiomers of CBCA-A are also referred to herein as “CBCA-1” and “CBCA-2.” In some embodiments, the method preferentially makes one enantiomer of CBCA-A, e.g., at a ratio of about 5:95, about 10:90, about 15:85, about 20:80, about 25:75, about 30:70, about 35:65, about 40:60, about 45:55, about 55:45, about 60:40, about 65:35, about 70:30, about 75:25, about 80:20, about 85:15, about 90:10, or about 95:5 of CBCA-1 to CBCA-2. In some embodiments, the method makes both enantiomers of CBCA-A.

In some embodiments, the disclosure provides a method of making CBCA, CBC, CBCOA, CBCVA, CBCO, CBCV, CBDA, CBD, CBDOA, CBDVA, CBDO, CBCV, THCA, THC, THCOA, THCVA, THCO, THCV, an isomer, analog or derivative thereof, or a combination thereof, comprising culturing the engineered cell comprising the non-natural flavin-dependent oxidase described herein, the polynucleotide described herein comprising the nucleic acid sequence encoding the non-natural flavin-dependent oxidase, the expression construct comprising the polynucleotide, or a combination thereof. In embodiments, the method makes CBCA, CBC, CBCOA, CBCVA, CBCO, CBCV, an isomer, analog or derivative thereof, or a combination thereof. In some embodiments, the method makes CBCA, e.g., one or both of CBCA-A and CBCA-B. In some embodiments, the disclosure provides a method of isolating CBCA, CBC, CBCOA, CBCVA, CBCO, CBCV, CBDA, CBD, CBDOA, CBDVA, CBDO, CBCV, THCA, THC, THCOA, THCVA, THCO, THCV, an isomer, analog or derivative thereof, or a combination thereof, from the cell extract or cell culture medium of the engineered cell. In embodiments, the method isolates CBCA, CBC, CBCOA, CBCVA, CBCO, CBCV, an isomer, analog or derivative thereof, or a combination thereof. In some embodiments, the method isolates CBCA. In some embodiments, the method isolates one or both regioisomers of CBCA, e.g., CBCA-A and CBCA-B. In some embodiments, the method preferentially isolates one enantiomer of CBCA-A, e.g., at a ratio of about 5:95, about 10:90, about 15:85, about 20:80, about 25:75, about 30:70, about 35:65, about 40:60, about 45:55, about 55:45, about 60:40, about 65:35, about 70:30, about 75:25, about 80:20, about 85:15, about 90:10, or about 95:5 of CBCA-1 to CBCA-2. In some embodiments, the method isolates both enantiomers of CBCA-A.

Methods of culturing cells, e.g., the engineered cell of the disclosure, are provided herein. Methods of isolating a cannabinoid, e.g., CBCA, CBC, CBCOA, CBCVA, CBCO, CBCV, CBDA, CBD, CBDOA, CBDVA, CBDO, CBDV, THCA, THC, THCOA, THCVA, THCO, THCV, an isomer, analog or derivative thereof, are also provided herein. In some embodiments, the cannabinoid is CBCA, CBC, CBCOA, CBCVA, CBCO, CBCV, an isomer, analog or derivative thereof, or a combination thereof. In some embodiments, the cannabinoid is CBCA. In some embodiments, the isolating comprises liquid-liquid extraction, pervaporation, evaporation, filtration, membrane filtration (including reverse osmosis, nanofiltration, ultrafiltration, and microfiltration), membrane filtration with diafiltration, membrane separation, reverse osmosis, electrodialysis, distillation, extractive distillation, reactive distillation, azeotropic distillation, crystallization and recrystallization, centrifugation, extractive filtration, ion exchange chromatography, size exclusion chromatography, adsorption chromatography, carbon adsorption, hydrogenation, ultrafiltration, or a combination thereof.

In some embodiments, the disclosure provides a method of making CBCA, CBDA, THCA, or an isomer, analog or derivative thereof, or a combination thereof, comprising contacting CBGA with the non-natural flavin-dependent oxidase described herein. In some embodiments, the disclosure provides a method of making CBCA, CBDA, THCA, or an isomer, analog or derivative thereof, or a combination thereof, comprising contacting CBGA with a flavin-dependent oxidase comprising any of SEQ ID NOs:1-3, any one of SEQ ID NOs:10-13, or any one of SEQ ID NOs:17-19. In some embodiments, the disclosure provides a method of making CBCA, CBDA, THCA, or an isomer, analog or derivative thereof, or a combination thereof, comprising contacting CBGA with Tcz9, Psx9, Svx9, Sox9, ThdO, ThnG, SrThX, Slx9, Frx9, Kcx9, or combination thereof. In some embodiments, the method makes CBCA or an isomer, analog or derivative thereof. In some embodiments, the method of making CBCA comprises contacting CBGA with a flavin-dependent oxidase comprising any one of SEQ ID NOs:1-3, SEQ ID NO:10, SEQ ID NO:11, SEQ ID NO:13, or SEQ ID NO:19. In some embodiments, the method of making CBCA comprises contacting CBGA with Tcz9, Psx9, Svx9, ThdO, SrThX, Slx9, Frx9, Kcx9, or combination thereof. In some embodiments, the method comprises making both enantiomers of CBCA shown in FIG. 2.

In some embodiments, the disclosure provides a method of making CBCOA, CBDOA, THCOA, or an isomer, analog or derivative thereof, or a combination thereof, comprising contacting CBGOA with the non-natural flavin-dependent oxidase described herein. In some embodiments, the disclosure provides a method of making CBCOA, CBDOA, THCOA, or an isomer, analog or derivative thereof, or a combination thereof, comprising contacting CBGOA with a flavin-dependent oxidase comprising any of SEQ ID NOs:1-3, any one of SEQ ID NOs:10-13, or any one of SEQ ID NOs:17-19. In some embodiments, the disclosure provides a method of making CBCOA, CBDOA, THCOA, or an isomer, analog or derivative thereof, or a combination thereof, comprising contacting CBGOA with Tcz9, Psx9, Svx9, Sox9, ThdO, ThnG, SrThX, Slx9, Frx9, Kcx9, or combination thereof.

In some embodiments, the disclosure provides a method of making CBCVA, CBDVA, THCVA, or an isomer, analog or derivative thereof, or a combination thereof, comprising contacting CBGVA with the non-natural flavin-dependent oxidase described herein. In some embodiments, the disclosure provides a method of making CBCVA, CBDVA, THCVA, or an isomer, analog or derivative thereof, or a combination thereof, comprising contacting CBGVA with a flavin-dependent oxidase comprising any of SEQ ID NOs:1-3, any one of SEQ ID NOs:10-13, or any one of SEQ ID NOs:17-19. In some embodiments, the disclosure provides a method of making CBCVA, CBDVA, THCVA, or an isomer, analog or derivative thereof, or a combination thereof, comprising contacting CBGVA with Tcz9, Psx9, Svx9, Sox9, ThdO, ThnG, SrThX, Slx9, Frx9, Kcx9, or combination thereof.

In some embodiments, the disclosure provides a method of making CBC, CBD, THC, or an isomer, analog or derivative thereof, or a combination thereof, comprising contacting CBG with the non-natural flavin-dependent oxidase described herein. In some embodiments, the disclosure provides a method of making CBC, CBD, THC, or an isomer, analog or derivative thereof, or a combination thereof, comprising contacting CBG with a flavin-dependent oxidase comprising any of SEQ ID NOs:1-3, any one of SEQ ID NOs:10-13, or any one of SEQ ID NOs:17-19. In some embodiments, the disclosure provides a method of making CBC, CBD, THC, or an isomer, analog or derivative thereof, or a combination thereof, comprising contacting CBG with Tcz9, Psx9, Svx9, Sox9, ThdO, ThnG, SrThX, Slx9, Frx9, Kcx9, or combination thereof.

In some embodiments, the disclosure provides a method of making CBCO, CBDO, THCO, or an isomer, analog or derivative thereof, or a combination thereof, comprising contacting CBGO with the non-natural flavin-dependent oxidase described herein. In some embodiments, the disclosure provides a method of making CBCO, CBDO, THCO, or an isomer, analog or derivative thereof, or a combination thereof, comprising contacting CBGO with a flavin-dependent oxidase comprising any of SEQ ID NOs:1-3, any one of SEQ ID NOs:10-13, or any one of SEQ ID NOs:17-19. In some embodiments, the disclosure provides a method of making CBCO, CBDO, THCO, or an isomer, analog or derivative thereof, or a combination thereof, comprising contacting CBGO with Tcz9, Psx9, Svx9, Sox9, ThdO, ThnG, SrThX, Slx9, Frx9, Kcx9, or combination thereof.

In some embodiments, the disclosure provides a method of making CBCV, CBDV, THCV, or an isomer, analog or derivative thereof, or a combination thereof, comprising contacting CBGV with the non-natural flavin-dependent oxidase described herein. In some embodiments, the disclosure provides a method of making CBCV, CBDV, THCV, or an isomer, analog or derivative thereof, or a combination thereof, comprising contacting CBGV with a flavin-dependent oxidase comprising any of SEQ ID NOs:1-3, any one of SEQ ID NOs:10-13, or any one of SEQ ID NOs:17-19. In some embodiments, the disclosure provides a method of making CBCV, CBDV, THCV, or an isomer, analog or derivative thereof, or a combination thereof, comprising contacting CBGV with Tcz9, Psx9, Svx9, Sox9, ThdO, ThnG, SrThX, Slx9, Frx9, Kcx9, or combination thereof.

In some embodiments, the flavin-dependent oxidase comprises SEQ ID NO:1. In some embodiments, the flavin-dependent oxidase comprises SEQ ID NO:2. In some embodiments, the flavin-dependent oxidase comprises SEQ ID NO:3. In some embodiments, the flavin-dependent oxidase comprises SEQ ID NO:10. In some embodiments, the flavin-dependent oxidase comprises SEQ ID NO:11. In some embodiments, the flavin-dependent oxidase comprises SEQ ID NO:12. In some embodiments, the flavin-dependent oxidase comprises SEQ ID NO:13. In some embodiments, the flavin-dependent oxidase comprises SEQ ID NO: 17. In some embodiments, the flavin-dependent oxidase comprises SEQ ID NO:18. In some embodiments, the flavin-dependent oxidase comprises SEQ ID NO:19. In some embodiments, the flavin-dependent oxidase comprise a polypeptide sequence comprising at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs:1-3, any one of SEQ ID NOs:10-13, or any one of SEQ ID NOs:17-19. In some embodiments, the flavin-dependent oxidase does not comprise a disulfide bond. In some embodiments, the flavin-dependent oxidase is not glycosylated. In some embodiments, the flavin-dependent oxidase has substantially the same catalytic activity at about pH 5 to about pH 8. In some embodiments, the flavin-dependent oxidase comprises a modification, e.g., an amino acid variation and/or a tag as described herein.

In some embodiments, the contacting occurs at about pH 4 to about pH 9, about pH 4.5 to about pH 8.5, about pH 5 to about pH 8, about pH 5.5 to about pH 7.5, or about pH 5 to about pH 7. In some embodiments, the method is performed in an in vitro reaction medium, e.g., an aqueous reaction medium. In some embodiments, the reaction medium further comprises a buffer, a salt, a surfactant, or a combination thereof. In some embodiments, the surfactant is about 0.005% (v/v) to about 5% (v/v) of the in vitro reaction medium. In some embodiments, the surfactant is about 0.01% (v/v) to about 1% (v/v) of the in vitro reaction medium. In some embodiments, the surfactant is about 0.05% (v/v) to about 0.5% (v/v) of the in vitro reaction medium. In some embodiments, the surfactant is about 0.08% (v/v) to about 0.2% (v/v) of the in vitro reaction medium. In some embodiments, the surfactant is a nonionic surfactant. Non-limiting examples of nonionic surfactants include TRITON™ X-100, TWEEN®, IGEPAL® CA-630, NONIDET™ P-40, and the like. In some embodiments, the surfactant is 2-[4-(2,4,4-trimethylpentan-2-yl)phenoxy]ethanol (also known as TRITON™ X-100). In some embodiments, the in vitro reaction medium comprises about 0.1% (v/v) 2-[4-(2,4,4-trimethylpentan-2-yl)phenoxy]ethanol.

In some embodiments, the non-natural flavin-dependent oxidase is produced by an engineered cell. In some embodiments, the non-natural flavin-dependent oxidase is overexpressed, e.g., on an exogenous nucleic acid such as a plasmid, by an inducible or constitutive promoter, in an engineered cell. In some embodiments, the disclosure provides a method of making an isolated non-natural flavin-dependent oxidase, comprising isolating the non-natural flavin-dependent oxidase expressed in the engineered cell. Methods of culturing cells, e.g., the engineered cell of the disclosure, are provided herein. In some embodiments, the disclosure provides an isolated non-natural flavin-dependent oxidase made by the methods provided herein.

Methods of isolating proteins (e.g., the non-natural flavin-dependent oxidase) from cells are known in the art. For example, the cells can be lysed to form a crude lysate, and the crude lysate can be further purified using filtration, centrifugation, chromatography, buffer exchange, or a combination thereof. The cell lysate is considered partially purified when about 10% to about 60%, or about 20% to about 50%, or about 30% to about 50% of the total proteins in the lysate is the desired protein of interest, e.g., the non-natural flavin-dependent oxidase. A protein can also be isolated from the cell lysate as a purified protein when greater than 60%, greater than 70%, greater than 80%, greater than 90%, greater than 95%, or greater than 99% of total proteins in the lysate is the desired protein of interest, e.g., the non-natural flavin-dependent oxidase.

In some embodiments, the crude lysate comprising the non-natural flavin-dependent oxidase is capable of converting CBGA to CBCA, CBDA, THCA, or an isomer, analog or derivative thereof. In some embodiments, the CBGA is contacted with crude cell lysate comprising the non-natural flavin-dependent oxidase to form CBCA, CBDA, THCA, or an isomer, analog or derivative thereof. In some embodiments, a partially purified lysate comprising the non-natural flavin-dependent oxidase is capable of converting CBGA to CBCA, CBDA, THCA, or an isomer, analog or derivative thereof. In some embodiments, the CBGA is contacted with partially purified lysate comprising the non-natural flavin-dependent oxidase to form CBCA, CBDA, THCA, or an isomer, analog or derivative thereof. In some embodiments, a purified non-natural flavin-dependent oxidase is capable of converting CBGA to CBCA, CBDA, THCA, or an isomer, analog or derivative thereof. In some embodiments, the CBGA is contacted with purified non-natural flavin-dependent oxidase to form CBCA, CBDA, THCA, or an isomer, analog or derivative thereof. In some embodiments, the CBGA is converted to CBCA or an isomer, analog or derivative thereof. In some embodiments, the CBGA is converted to THCA or an isomer, analog or derivative thereof. In some embodiments, the non-natural flavin-dependent oxidase comprises at least 90% sequence identity to any one of SEQ ID NOs:1-3, SEQ ID NO:10, SEQ ID NO:11, SEQ ID NO:13, or SEQ ID NO:19.

In some embodiments, the crude lysate comprising the non-natural flavin-dependent oxidase is capable of converting CBGOA to CBCOA, CBDOA, THCOA, or an isomer, analog or derivative thereof. In some embodiments, the CBGOA is contacted with crude cell lysate comprising the non-natural flavin-dependent oxidase to form CBCOA, CBDOA, THCOA, or an isomer, analog or derivative thereof. In some embodiments, a partially purified lysate comprising the non-natural flavin-dependent oxidase is capable of converting CBGOA to CBCOA, CBDOA, THCOA, or an isomer, analog or derivative thereof. In some embodiments, the CBGOA is contacted with partially purified lysate comprising the non-natural flavin-dependent oxidase to form CBCOA, CBDOA, THCOA, or an isomer, analog or derivative thereof. In some embodiments, a purified non-natural flavin-dependent oxidase is capable of converting CBGOA to CBCOA, CBDOA, THCOA, or an isomer, analog or derivative thereof. In some embodiments, the CBGOA is contacted with purified non-natural flavin-dependent oxidase to form CBCOA, CBDOA, THCOA, or an isomer, analog or derivative thereof. In some embodiments, the CBGOA is converted to CBCOA or an isomer, analog or derivative thereof.

In some embodiments, the crude lysate comprising the non-natural flavin-dependent oxidase is capable of converting CBGVA to CBCVA, CBDVA, THCVA, or an isomer, analog or derivative thereof. In some embodiments, the CBGVA is contacted with crude cell lysate comprising the non-natural flavin-dependent oxidase to form CBCVA, CBDVA, THCVA, or an isomer, analog or derivative thereof. In some embodiments, a partially purified lysate comprising the non-natural flavin-dependent oxidase is capable of converting CBGVA to CBCVA, CBDVA, THCVA, or an isomer, analog or derivative thereof. In some embodiments, the CBGVA is contacted with partially purified lysate comprising the non-natural flavin-dependent oxidase to form CBCVA, CBDVA, THCVA, or an isomer, analog or derivative thereof. In some embodiments, a purified non-natural flavin-dependent oxidase is capable of converting CBGVA to CBCVA, CBDVA, THCVA, or an isomer, analog or derivative thereof. In some embodiments, the CBGVA is contacted with purified non-natural flavin-dependent oxidase to form CBCVA, CBDVA, THCVA, or an isomer, analog or derivative thereof. In some embodiments, the CBGVA is converted to CBCVA or an isomer, analog or derivative thereof.

In some embodiments, the crude lysate comprising the non-natural flavin-dependent oxidase is capable of converting CBGO to CBCO, CBDO, THCO, or an isomer, analog or derivative thereof. In some embodiments, the CBGO is contacted with crude cell lysate comprising the non-natural flavin-dependent oxidase to form CBCO, CBDO, THCO, or an isomer, analog or derivative thereof. In some embodiments, a partially purified lysate comprising the non-natural flavin-dependent oxidase is capable of converting CBGO to CBCO, CBDO, THCO, or an isomer, analog or derivative thereof. In some embodiments, the CBGO is contacted with partially purified lysate comprising the non-natural flavin-dependent oxidase to form CBCO, CBDO, THCO, or an isomer, analog or derivative thereof. In some embodiments, a purified non-natural flavin-dependent oxidase is capable of converting CBGO to CBCO, CBDO, THCO, or an isomer, analog or derivative thereof. In some embodiments, the CBGO is contacted with purified non-natural flavin-dependent oxidase to form CBCO, CBDO, THCO, or an isomer, analog or derivative thereof. In some embodiments, the CBGO is converted to CBCO or an isomer, analog or derivative thereof.

In some embodiments, the crude lysate comprising the non-natural flavin-dependent oxidase is capable of converting CBGV to CBCV, CBDV, THCV, or an isomer, analog or derivative thereof. In some embodiments, the CBGV is contacted with crude cell lysate comprising the non-natural flavin-dependent oxidase to form CBCV, CBDV, THCV, or an isomer, analog or derivative thereof. In some embodiments, a partially purified lysate comprising the non-natural flavin-dependent oxidase is capable of converting CBGV to CBCV, CBDV, THCV, or an isomer, analog or derivative thereof. In some embodiments, the CBGV is contacted with partially purified lysate comprising the non-natural flavin-dependent oxidase to form CBCV, CBDV, THCV, or an isomer, analog or derivative thereof. In some embodiments, a purified non-natural flavin-dependent oxidase is capable of converting CBGV to CBCV, CBDV, THCV, or an isomer, analog or derivative thereof. In some embodiments, the CBGV is contacted with purified non-natural flavin-dependent oxidase to form CBCV, CBDV, THCV, or an isomer, analog or derivative thereof. In some embodiments, the CBGV is converted to CBCV or an isomer, analog or derivative thereof.

In some embodiments, the crude lysate comprising the non-natural flavin-dependent oxidase is capable of converting CBG to CBC, CBD, THC, or an isomer, analog or derivative thereof. In some embodiments, the CBG is contacted with crude cell lysate comprising the non-natural flavin-dependent oxidase to form CBC, CBD, THC, or an isomer, analog or derivative thereof. In some embodiments, a partially purified lysate comprising the non-natural flavin-dependent oxidase is capable of converting CBG to CBC, CBD, THC, or an isomer, analog or derivative thereof. In some embodiments, the CBG is contacted with partially purified lysate comprising the non-natural flavin-dependent oxidase to form CBC, CBD, THC, or an isomer, analog or derivative thereof. In some embodiments, a purified non-natural flavin-dependent oxidase is capable of converting CBG to CBC, CBD, THC, or an isomer, analog or derivative thereof. In some embodiments, the CBG is contacted with purified non-natural flavin-dependent oxidase to form CBC, CBD, THC, or an isomer, analog or derivative thereof. In some embodiments, the CBG is converted to CBC or an isomer, analog or derivative thereof.

### Compositions

In some embodiments, the disclosure provides a composition comprising a cannabinoid or an isomer, analog or derivative thereof obtained from the engineered cell, cell extract, or method described herein. In some embodiments, the cannabinoid is CBCA, CBDA, THCA, CBCOA, CBDOA, THCOA, CBCVA, CBDVA, THCVA, CBC, CBD, THC, CBCO, CBDO, THCO, CBCV, CBDV, THCV, or an isomer, analog or derivative thereof, or a combination thereof. In some embodiments, the cannabinoid is CBCA, CBCOA, CBCVA, CBC, CBCO, CBCV, or an isomer, analog or derivative thereof, or a combination thereof. In some embodiments, the cannabinoid is CBCA.

In some embodiments, the cannabinoid is 10% or greater, 20% or greater, 30% or greater, 40% or greater, 50% or greater, 60% or greater, 70% or greater, 80% or greater, 85% or greater, 90% or greater, 91% or greater, 92% or greater, 93% or greater, 94% or greater, 95% or greater, 96% or greater, 97% or greater, 98% or greater, 99% or greater, 99.2% or greater, 99.4% or greater, 99.5% or greater, 99.6% or greater, 99.7% or greater, 99.8% or greater, or 99.9% or greater of total cannabinoid compound(s) in the composition. In some embodiments, the cannabinoid is CBCA or an isomer, analog or derivative thereof. In some embodiments, the cannabinoid is THCA or an isomer, analog or derivative thereof. In some embodiments, the cannabinoid is CBCOA or an isomer, analog or derivative thereof. In some embodiments, the cannabinoid is CBCVA or an isomer, analog or derivative thereof. In some embodiments, the cannabinoid is CBC or an isomer, analog or derivative thereof. In some embodiments, the cannabinoid is CBCO or an isomer, analog or derivative thereof. In some embodiments, the cannabinoid is CBCV or an isomer, analog or derivative thereof. In some embodiments, the cannabinoid comprises any combination of CBCA, CBDA, THCA, CBCOA, CBDOA, THCOA, CBCVA, CBDVA, THCVA, CBC, CBD, THC, CBCO, CBDO, THCO, CBCV, CBDV, THCV, or an isomer, analog, or derivative thereof. In some embodiments, the cannabinoid comprises any combination of CBCA, CBCOA, CBCVA, CBC, or an isomer, analog, or derivative thereof. In some embodiments, the cannabinoid comprises CBCA.

In some embodiments, the composition is a therapeutic or medicinal composition. In some embodiments, the composition further comprises a pharmaceutically acceptable excipient. In some embodiments, the composition is a topical composition. In some embodiments, the composition is in the form of a cream, a lotion, a paste, or an ointment.

In some embodiments, the composition is an edible composition. In some embodiments, the composition is provided in a food or beverage product. In some embodiments, the composition is an oral unit dosage composition. In some embodiments, the composition is provided in a tablet or a capsule.

In some embodiments, the disclosure provides a composition comprising (a) a non-natural flavin-dependent oxidase as described herein; and (b) a cannabinoid, a prenylated aromatic compound, or both. In some embodiments, the cannabinoid is CBCA, CBDA, THCA, CBCOA, CBDOA, THCOA, CBCVA, CBDVA, THCVA, CBC, CBD, THC, CBCO, CBDO, THCO, CBCV, CBDV, THCV, or an isomer, analog, or derivative thereof, or a combination thereof. In some embodiments, the cannabinoid or prenylated aromatic compound is CBGA, CBCA, CBGOA, CBCOA, CBGVA, CBCVA, CBG, CBC, CBGO, CBCO, CBGV, CBCV, or an isomer, analog, or derivative thereof, or a combination thereof. In some embodiments, the cannabinoid or prenylated aromatic compound comprises CBGA, CBCA, an isomer, analog, or derivative thereof, or a combination thereof.

In some embodiments, the disclosure provides a composition comprising (a) a flavin-dependent oxidase comprising any one of SEQ ID NOs:1-3, any one of SEQ ID NOs:10-13, or any one of SEQ ID NOs:17-19; and (b) a cannabinoid, a prenylated aromatic compound, or both. In some embodiments, the flavin-dependent oxidase comprises SEQ ID NO:1. In some embodiments, the flavin-dependent oxidase comprises SEQ ID NO:2. In some embodiments, the flavin-dependent oxidase comprises SEQ ID NO:3. In some embodiments, the flavin-dependent oxidase comprises SEQ ID NO:10. In some embodiments, the flavin-dependent oxidase comprises SEQ ID NO:11. In some embodiments, the flavin-dependent oxidase comprises SEQ ID NO:12. In some embodiments, the flavin-dependent oxidase comprises SEQ ID NO:13. In some embodiments, the flavin-dependent oxidase comprises SEQ ID NO:17. In some embodiments, the flavin-dependent oxidase comprises SEQ ID NO:18. In some embodiments, the flavin-dependent oxidase comprises SEQ ID NO:19. In some embodiments, the flavin-dependent oxidase is Tcz9. In some embodiments, the flavin-dependent oxidase is Psx9. In some embodiments, the flavin-dependent oxidase is Svx9. In some embodiments, the flavin-dependent oxidase is Sox9. In some embodiments, the flavin-dependent oxidase is ThdO. In some embodiments, the flavin-dependent oxidase is ThnG. In some embodiments, the flavin-dependent oxidase is SrThX. In some embodiments, the flavin-dependent oxidase is Slx9. In some embodiments, the flavin-dependent oxidase is Frx9. In some embodiments, the flavin-dependent oxidase is Kcx9. In some embodiments, the cannabinoid or prenylated aromatic compound is CBGA, CBGOA, CBGVA, CBG, CBGO, CBGV, CBCA, CBDA, THCA, CBCOA, CBDOA, THCOA, CBCVA, CBDVA, THCVA, CBC, CBD, THC, CBCO, CBDO, THCO, CBCV, CBDV, THCV, or an isomer, analog, or derivative thereof, or a combination thereof. In some embodiments, the cannabinoid or prenylated aromatic compound is CBGA, CBCA, CBGOA, CBCOA, CBGVA, CBCVA, CBG, CBC, CBGO, CBCO, CBGV, CBCV, or an isomer, analog, or derivative thereof, or a combination thereof. In some embodiments, the cannabinoid or prenylated aromatic compound comprises CBGA, CBCA (e.g., CBCA-A and/or CBCA-B), an isomer, analog, or derivative thereof, or a combination thereof.

In some embodiments, the compositions herein comprising a flavin-dependent oxidase and a cannabinoid, a prenylated aromatic compound, or both, further comprise an enzyme in a cannabinoid biosynthesis pathway. Cannabinoid biosynthesis pathways are described herein. In some embodiments, the cannabinoid biosynthesis pathway enzyme comprises olivetol synthase (OLS), olivetolic acid cyclase (OAC), prenyltransferase, or a combination thereof.

All references cited herein, including patents, patent applications, papers, textbooks and the like, and the references cited therein, to the extent that they are not already, are hereby incorporated herein by reference in their entirety.

### EXAMPLES

### Example 1. Cannabinoid Synthase Activity of Tcz9, Psx9, and Svx9

The enzymes listed in Table 3 were evaluated as cannabinoid synthases:

TABLE 3
Potential cannabinoid synthases

| Accession number | Name | Organism | SEQ ID |
|---|---|---|---|
| WP_180931280.1 | Tcz9 | Streptomyces sp. AJS327 | 1 |
| WP_173153368.1 | Psx9 | Phytohabitans suffuscus | 2 |
| WP_030877494.1 | Svx9 | Streptomyces varsoviensis | 3 |

Tcz9 is involved in tetrachlorizine biosynthesis in Streptomyces sp. AJS327 (Purdy et al. 2021, J Am Chem Soc 143(10):3682-3686 (2021)) and the other two enzymes are part of an unknown biosynthetic gene cluster in Phytohabitans suffuscus and Streptomyces varsoviensis that resemble the tetrachlorizine gene cluster.

Synthetic genes with optimized codons for expression in E. coli were cloned with and without an N-terminal 6×poly-histidine tag into a low-copy expression vector (Psc101 replicon, carbenicillin resistance marker) with an inducible or constitutive promoter. The expression plasmids were transformed into E. coli BL21 and tested with CBGA in an in-vitro lysate assay with CBGA as substrate.

Single colonies were used to inoculate 2 Ml cultures of LB media containing carbenicillin and allowed to grow for 18 hours at room temperature with shaking. 20 μLs of the starter cultures were used to inoculate 2 Ml of fresh LB/carb media. Cultures containing inducible plasmids were allowed to grow at 35° C. until an OD₆₀₀=0.6 was reached, then 1 Mm IPTG and 200 μM cumate was added to the cultures to induce protein expression. Protein was allowed to express for 20 hours at room temperature. Plasmids under constitutive induction were dilute 100-fold into fresh LB/carb media and allowed to grow and express protein for 21 hours at room temperature. Cultures were harvested by centrifugation and frozen until needed.

Frozen cell pellets were resuspended to an OD₆₀₀=5 in 50 Mm HEPES/HCl, pH 7.4 containing 50% BPER II with 2 U/μL of lysozyme and benzonase. Cell lysis proceeded at room temperature for 10 mins. Cell debris was pelleted by centrifugation. 4 μLs of clarified lysate was used for the in vitro reaction with CBGA.

Analytical Methods. The enzymes were tested for their ability to produce cannabinoids from CBGA in 100 Mm Tris buffer, pH 7.4, or 100 Mm Citrate buffer, pH 5.0. Cannabinoid products were detected using HPLC/MS/MS following the mass of 357 Da (M-1) and three common cannabinoid fragments: loss of water (−18 Da, 339 Da), loss of carboxylic acid (−44 Da, 313 Da), and loss of carboxylic acid (−44 Da) and terpenyl C9 side chain (−122 Da) yielding peak at 191 Da.

The structures of cannabinoid products observed in the enzymatic reactions were confirmed by LC/MS/MS (Thermo Vanquish™ LC). LC used the following conditions: A solvent gradient was performed as follows: Starting with 15% solvent B for 1 minute, then ramp up solvent B to 95% until 12 minutes and hold for 3 minutes until 15 minutes, then a sharp return to 15% solvent B over a 0.1 minute period until 15.1 minutes, and re-equilibrate until 18 minutes. The whole gradient takes 18 minutes, and the column temperature is set at 40° C. (Solvent A: 0.1% Ammonium Acetate in H₂O, Solvent B: Acetonitrile). MS data was acquired using heated electrospray sample introduction method with subsequent ion detection and separation using Thermo Q Exactive™ instrument. Data was acquired using Polarity switching to get coverage for both positive and negative ions using identical settings. The acquired MS data used following settings: AGC target was set at 1.00×10⁶ ions with minimum IT of 100 ms over 1 microscan and the m/z range of 70 to 1050 m/z at 30000 resolution. The acquired MS/MS data used the following settings: AGC target was set at 1.00×10⁵ with dynamic ion exclusion over 3.0 sec with minimum IT of 80 ms over 1 microscan and the isolation window of 1 m/z at 17500 resolution. Normalized collision energy was stepped at 25, 35 and 45 Ev.

Results. All three of the tested enzymes converted CBGA to CBCA. Table 4 shows the amount of CBCA formed in 24 hours at 37° C. The highest amount of CBCA was formed with the lysate containing Psx9.

TABLE 4
CBCA Formation

| | | | CBCA | CBCA |
|---|---|---|---|---|
| | | | (μM) | (μM) |
| | | | at pH | at pH |
| Organism | Enzyme | Expression | 7.4 | 5.0 |
| Streptomyces sp. AJS327 | Tcz9 | Inducible | 24.1 | 8.7 |
| Phytohabitans suffuscus | Psx9 | Inducible | 123.3 | 190.2 |
| Streptomyces varsoviensis | Svx9 | Inducible | 101.3 | 28.2 |
| Streptomyces sp. AJS327 | Tcz9 | Constitutive | N.D. | N.D. |
| Phytohabitans suffuscus | Psx9 | Constitutive | 157.0 | 218.9 |
| Streptomyces varsoviensis | Svx9 | Constitutive | 103.3 | 23.8 |

(N.D. = not detected.)

### Example 2. Cannabinoid Synthase Activity of Sox9, ThdO, ThnG, SrThX, Slx9, Frx9, and Kcx9

The enzymes listed in Table 5 were evaluated as cannabinoid synthases:

TABLE 5
Potential cannabinoid synthases

| Accession number | Name | Organism | SEQ ID |
|---|---|---|---|
| WP_235454663.1 | Sox9 | Streptomyces olivochromogenes | 10 |
| ANW12117.1 | ThdO | Streptomyces albogriseolus | 11 |
| WP_067055472.1 | ThnG | Streptomyces sp. FXJ1.172 | 12 |
| WP_098755161.1 | SrThX | Streptomyces sp. Ru87 | 13 |
| WP_156205671.1 | Slx9 | Streptomyces lycii | 17 |
| WP_239335163.1 | Frx9 | Frankia sp. CiP 3 | 18 |
| WP_211768552.1 | Kcx9 | Kutzneria sp. CA-103260 | 19 |

TdO and ThnG are associated with the thienodolin biosynthesis gene cluster in Streptomyces albogriseolus and Streptomyces sp. FXJ1.172, respectively. SrThX is a homolog of ThdO and ThnG from an unknown thienodolin biosynthesis gene cluster in Streptomyces sp. Ru87. Slx9, Frx9, and Kcx9 were identified from a further search.

Synthetic genes were cloned, transformed in E. coli BL21, and tested with CBGA in an in vitro lysate assay in the same manner as Example 1.

The analytical methods for identifying conversion products of CBGA were the same as Example 1. The in vitro conversion of CBGA was evaluated at pH 5.0 and at pH 7.4 with 200 μM CBGA (ThdO, ThnG, and SrThX) or 500 μM CBGA (Sox9, Slx9, Frx9, and Kcx9) in the presence of Triton-X100 as solubilizing agent. Table 6 shows the amount of CBCA formed by the tested enzymes in 24 hours at 37° C. Table 6 demonstrates that Sox9, ThdO, SrThX, and Kcx9 had cannabinoid synthase activity under the tested conditions.

TABLE 6
CBCA Formation

| | | | CBCA | CBCA |
|---|---|---|---|---|
| | | | (μM) | (μM) |
| | | Ex- | at pH | at pH |
| Organism | Enzyme | pression | 7.4 | 5.0 |
| Streptomyces olivochromogenes | Sox9 | Inducible | 271.5 | 36.7 |
| Streptomyces albogriseolus | ThdO | Inducible | 11.8 | 0.9 |
| Streptomyces sp. FXJ1.172 | ThnG | Inducible | N.D. | N.D. |
| Streptomyces sp. Ru87 | SrThX | Inducible | 0.7 | N.D. |
| Streptomyces lycii | Slx9 | Inducible | N.D. | N.D. |
| Frankia sp. CiP3 | Frx9 | Inducible | N.D. | N.D. |
| Kutzneria sp. CA-103260 | Kcx9 | Inducible | 12.9 | 9.0 |

(N.D. = not detected.)

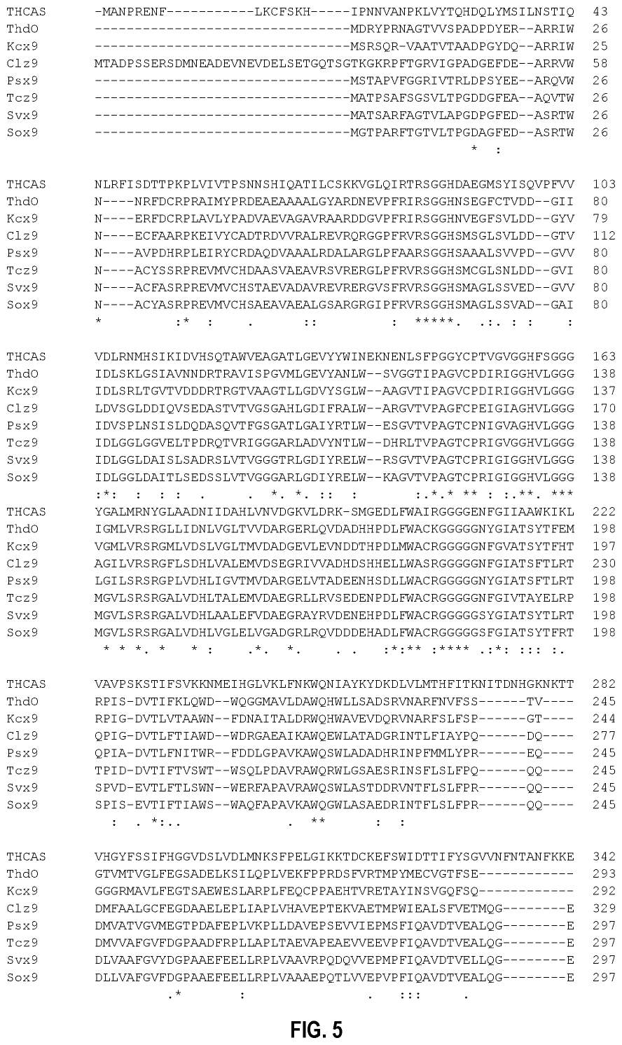

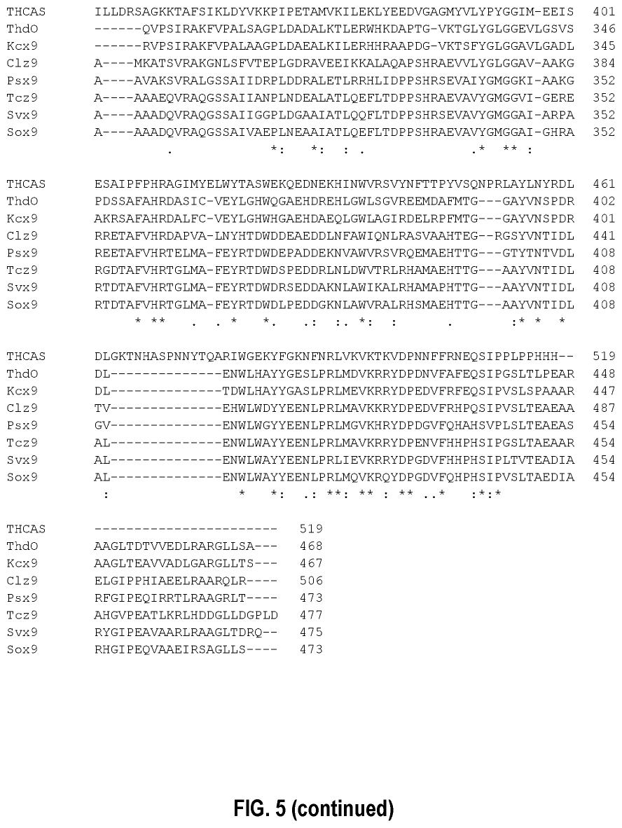

The LC/MS spectra of the Sox9 reaction with CBGA is shown in FIG. 4. The top and middle panels of FIG. 4 show the LC/MS spectra of a CBDA standard and a CBCA-A standard, respectively. The bottom panel of FIG. 4 shows the LC/MS spectra of the reaction products with Sox9 and CBGA. The peak at 1.46 min corresponds to CBCA-A. The peak at 0.88 min may correspond to CBDA.

### Example 3. Evaluation of Purified Enzymes for Activity and Entioselectivity

Purified Tcz9, Psx9, Svx9, and ThdO were evaluated for activity and enantioselectivity. The enzymes were expressed as described above for the previous Examples. The cells were lysed using a microfluidizer, and the enzymes were purified using a HisTrap™ nickel affinity column.

The in vitro conversion of CBGA with the purified enzymes was evaluated at pH 7.4 and at pH 5.0 with 200 μM CBGA in the presence of DMSO and Triton-X100 as solubilizing agent. Table 7 shows the amount of CBCA formed in 24 hours at 37° C. ThdO produced the most CBCA at pH 7.4, while Psx9 produced the most CBCA at pH 5.0.

TABLE 7
CBCA Formation

| | | CBCA (μM) | CBCA (μM) |
|---|---|---|---|
| Organism | Enzyme | at pH 7.4 | at pH 5.0 |
| Phytohabitans suffuscus | Psx9 | 173.0 | 105.31 |
| Streptomyces sp. AJS327 | Tcz9 | 70.3 | 32.3 |
| Streptomyces varsoviensis | Svx9 | 145.1 | 100.8 |
| Streptomyces albogriseolus | ThdO | 182.5 | 58.6 |

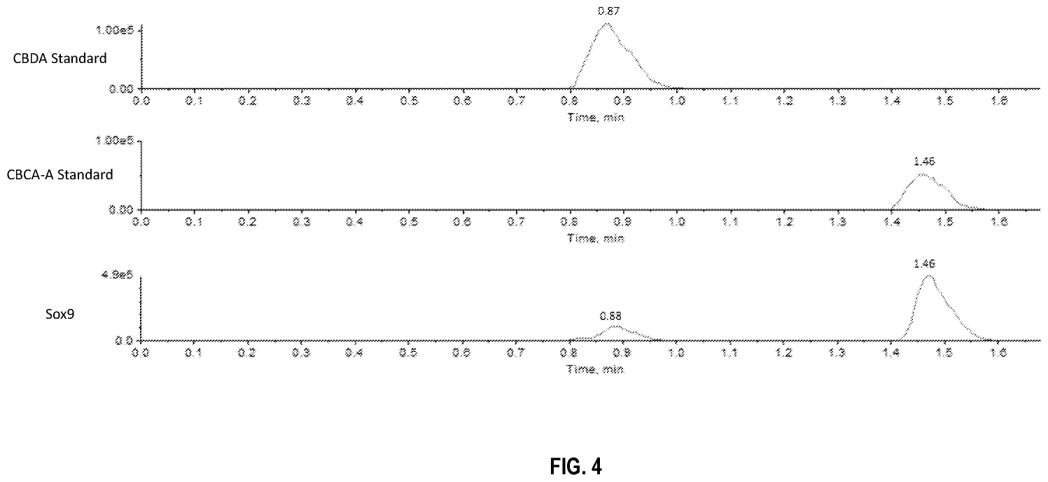

Purified Tcz9, Psx9, Svx9, and ThdO were further evaluated for enantioselective cyclization to the two CBCA enantiomers (FIG. 2) at pH 7.4 and pH 5.0. The Clz9 enzyme from Streptomyces sp. CNH-287 was also evaluated at pH 7.4. Enantiomers were detected by chiral HPLC chromatography using a Daicel CHIRALPAK® AD-H HPLC analytical column (5 μm, 4.6 mm×250 mm). A racemic mix of CBCA was used as an external standard with the slower moving enantiomer designated as CBCA-1 (retention time of 10.1 min) and the faster moving enantiomer designated as CBCA-2 (retention time of 18.2 min) (see FIG. 3A). It was not determined which CBGA enantiomer structures in FIG. 2 correspond to CBCA-1 and CBCA-2.

As shown in Tables 8 and 9, the tested enzymes produced a near-racemic mixture of CBCA-1 and CBCA-2 at pH 7.4 (Table 8) and pH 5.0 (Table 9). Psx9, Tcz9, and Svx9 exhibited greater enantioselectivity for production of CBCA-2 at pH 7.4 and pH 5.0. Clz9 favored enantioselective production of CBCA-1 at pH 7.4, and ThdO favored enantioselective production of CBCA-1 at pH 7.4 and pH 5.0. FIG. 3B shows the HPLC chromatogram of the in vitro reaction of Psx9 with CBGA as substrate at pH 7.4.

TABLE 8
CBCA Enantiomers Formation at pH 7.4

| | | CBCA-1 | CBCA-2 | % CBCA-1:% |
|---|---|---|---|---|
| Organism | Enzyme | (μM) | (μM) | CBCA-2 |
| Streptomyces | Clz9 | 20.8 | 13.4 | 60.8:39.2 |
| sp. CNH-287 | | | | |
| Phytohabitans | Psx9 | 78.3 | 94.6 | 45.3:54.7 |
| suffuscus | | | | |
| Streptomyces | Tcz9 | 26.8 | 43.5 | 38.1:61.9 |
| sp. AJS327 | | | | |
| Streptomyces | ThdO | 98.9 | 83.6 | 54.2:45.8 |
| albogriseolus | | | | |
| Streptomyces | Svx9 | 68.8 | 76.3 | 47.4:52.6 |
| varsoviensis | | | | |

TABLE 9
CBCA Enantiomers Formation at pH 5.0

| | | CBCA-1 | CBCA-2 | % CBCA-1:% |
|---|---|---|---|---|
| Organism | Enzyme | (μM) | (μM) | CBCA-2 |
| Phytohabitans | Psx9 | 18.0 | 87.3 | 17.1:82.9 |
| suffuscus | | | | |
| Streptomyces | Tcz9 | 4.7 | 27.6 | 14.5:85.5 |
| sp. AJS327 | | | | |
| Streptomyces | ThdO | 35.6 | 22.9 | 60.8:39.2 |
| albogriseolus | | | | |
| Streptomyces | Svx9 | 15.5 | 85.3 | 15.4:84.6 |
| varsoviensis | | | | |

Sequences

SEQ ID NO: 1-Accession ID No. WP_180931280.1 from Streptomyces sp. AJS327
(″Tcz9″)
MATPSAFSGSVLTPGDDGFEAAQVTWNACYSSRPREVMVCHDAASVAEAVRSVRERGLPFRVRSGGHSMCGLSNLDDGVIIDLGGLG
GVELTPDRQTVRIGGGARLADVYNTLWDHRLTVPAGTCPRIGVGGHVLGGGMGVLSRSRGALVDHLTALEMVDAEGRLLRVSEDENP
DLFWACRGGGGGNFGIVTAYELRPTPIDDVTIFTVSWTWSQLPDAVRAWQRWLGSAESRINSFLSLFPQQQDMVVAFGVFDGPAADF
RPLLAPLTAEVAPEAEVVEEVPFIQAVDTVEALQGEAAAAEQVRAQGSSAIIANPLNDEALATLQEFLTDPPSHRAEVAVYGMGGVI
GERERGDTAFVHRTGLMAFEYRTDWDSPEDDRLNLDWVTRLRHAMAEHTTGAAYVNTIDLALENWLWAYYEENLPRLMAVKRRYDPE
NVFHHPHSIPGSLTAEAARAHGVPEATLKRLHDDGLLDGPLD
SEQ ID NO: 2-Accession ID No. WP_173153368.1 from Phytohabitans suffuscus
(″Psx9″)
MSTAPVFGGRIVTRLDPSYEEARQVWNAVPDHRPLEIRYCRDAQDVAAALRDALARGLPFAARSGGHSAAALSVVPDGVVIDVSPLN
SISLDQDASQVTFGSGATLGAIYRTLWESGVTVPAGTCPNIGVAGHVLGGGLGILSRSRGPLVDHLIGVTMVDARGELVTADEENHS
DLLWACRGGGGGNYGIATSFTLRTQPIADVTLFNITWRFDDLGPAVKAWQSWLADADHRINPFMMLYPREQDMVATVGVMEGTPDAF
EPLVKPLLDAVEPSEVVIEPMSFIQAVDTVEALQGEAAVAKSVRALGSSAIIDRPLDDRALETLRRHLIDPPSHRSEVAIYGMGGKI
AAKGREETAFVHRTELMAFEYRTDWDEPADDEKNVAWVRSVRQEMAEHTTGGTYTNTVDLGVENWLWGYYEENLPRLMGVKHRYDPD
GVFQHAHSVPLSLTEAEASRFGIPEQIRRTLRAAGRLT
SEQ ID NO: 3-Accession ID No. WP_030877494.1 from Streptomyces varsoviensis
(″Svx9″)
MATSARFAGTVLAPGDPGFEDASRTWNACFASRPREVMVCHSTAEVADAVREVRERGVSFRVRSGGHSMAGLSSVEDGVVIDLGGLD
AISLSADRSLVTVGGGTRLGDIYRELWRSGVTVPAGTCPRIGIGGHVLGGGMGVLSRSRGALVDHLAALEFVDAEGRAYRVDENEHP
DLFWACRGGGGGSYGIATSYTLRTSPVDEVTLFTLSWNWERFAPAVRAWQSWLASTDDRVNTFLSLFPRQQDLVAAFGVYDGPAAEF
EELLRPLVAAVRPQDQVVEPMPFIQAVDTVELLQGEAAAADQVRAQGSSAIIGGPLDGAAIATLQQFLTDPPSHRSEVAVYGMGGAI
ARPARTDTAFVHRTGLMAFEYRTDWDRSEDDAKNLAWIKALRHAMAPHTTGAAYVNTIDLALENWLWAYYEENLPRLIEVKRRYDPG
DVFHHPHSIPLTVTEADIARYGIPEAVAARLRAAGLTDRQ
SEQ ID NO: 4-Polynucleotide encoding SEQ ID NO: 2 (Psx9)
atgagcaccgcgcccgttttcggcgggaggatcgtcacccggctggacccctcgtacgaggaggcccgccaggtgtggaacgccgtg
cccgaccaccgtccacttgagatcaggtactgccgggacgcgcaggacgtggcggcggcgctgcgcgacgcgctggcgcggggcctg
ccgttcgccgcccgctcgggtgggcactcggcggccgccctgtcagtggtcccagacggcgtggtgatcgacgtctcgccgttgaac
tcgatcagcctcgaccaggacgcctcgcaggtgaccttcggcagcggtgcgacgctcggcgccatctaccgcacgctgtgggagtcg
ggcgtgaccgtaccggcgggcacgtgcccgaacatcggcgtcgcgggccacgtgctcggcggtggcctgggcatcctctcccgctcg
cgcggcccgctcgtcgatcacctgatcggggtgacgatggtcgacgcgcgcggcgagcttgtcacggcggacgaggagaaccactcg
gatctgctgtgggcctgccgtggtggcgggggtggcaactacggcatcgccacctcgttcacccttcgaacccagcccatcgccgat
gtcaccctgttcaacatcacctggcggttcgacgacctcggccccgcggtaaaggcatggcagagctggctggccgacgccgaccac
cggatcaacccgttcatgatgctctacccgcgcgagcaggacatggtcgccacggtgggtgtcatggagggcacgcccgacgcgttc
gagccgttggtcaagccgttgttggacgccgtggagccgtccgaggtggtcatcgagccgatgtcgttcatccaggcggtggacacc
gtcgaggcgttgcagggagaggcggctgtcgccaagtccgtccgggcgctgggcagctcggcgatcatcgatcggccgctggacgac
cgggcccttgagaccctgcggcgccacctgatcgacccgcccagtcaccgaagtgaggtggcgatctacggcatgggcgggaagatc
gccgccaagggtcgcgaggagacggcgttcgtgcaccgtacggagctgatggccttcgagtaccgcaccgactgggacgagccggcg
gacgacgagaagaacgtggcctgggtgcggagcgtacggcaggagatggccgagcacaccaccggtggcacgtacacgaacaccgtc
gatctcggggtcgagaactggctgtggggctactacgaggagaacctgccgcggttgatgggggtcaagcaccgctacgaccccgac
ggggtcttccaacatgcccacagcgtcccgctgtcgttgaccgaggcggaggcgagccgcttcgggattccagagcagatccgccgg
accctacgcgcggccggccgcctgacctga
SEQ ID NO: 5-Polynucleotide encoding SEQ ID NO: 3 (Svx9)
ATGGCAACCTCCGCACGTTTCGCCGGTACGGTCCTCGCCCCCGGGGACCCCGGATTCGAGGACGCGAGCCGCACCTGGAACGCGTGC
TTCGCCTCCCGCCCCCGCGAGGTGATGGTGTGTCACAGCACGGCGGAGGTGGCGGACGCGGTGCGCGAGGTGCGGGAGCGCGGCGTA
TCGTTCCGGGTCCGGTCGGGAGGCCACTCCATGGCCGGGCTGAGCAGCGTCGAGGACGGGGTCGTCATCGACCTCGGCGGCCTCGAC
GCCATCTCCCTCAGCGCGGACCGCTCGCTGGTGACCGTCGGCGGCGGAACCCGACTCGGCGACATCTACCGGGAGTIGTGGCGGTCC
GGCGTGACGGTACCGGCGGGCACCTGCCCGCGCATCGGCATCGGCGGCCATGTGCTGGGCGGCGGCATGGGCGTGCTGTCCCGCTCC
CGCGGCGCCCTCGTCGACCACCTCGCCGCCCTGGAGTTCGTCGACGCCGAGGGCCGGGCGTACCGGGTGGACGAGAACGAGCACCCC
GACCTGTTCTGGGCCTGCCGGGGcGGGGGGGGCGGGAGCTACGGCATCGCCACCTCGTACACGCTGCGCACGTCCCCGGTGGACGAG
GTCACCCTCTTCACCCTGAGCTGGAACTGGGAACGGTTCGCCCCCGCCGTACGGGCGTGGCAGAGCTGGCTGGCGTCGACCGACGAC
CGGGTCAACACCTTCCTCTCGCTCTTCCCGCGCCAGCAGGACCTGGTGGCGGCCTTCGGCGTGTACGACGGGCCGGCCGCCGAGTTC
GAGGAGCTGCTGCGGCCGCTGGTCGCCGCCGTACGGCCGCAGGACCAGGTCGTGGAGCCGATGCCGTTCATCCAGGCCGTCGACACC
GTGGAACTGCTCCAGGGCGAGGCCGCGGCGGCCGACCAGGTACGGGCGCAGGGCAGTTCGGCCATCATCGGCGGGCCGCTGGACGGC
GCGGCCATCGCCACCCTCCAGCAGTTCCTCACCGACCCGCCGAGCCACCGCTCCGAGGTCGCCGTCTACGGCATGGGGGGGGCCATC
GCGCGCCCGGCGCGCACCGACACGGCGTTCGTGCACCGGACGGGGCTGATGGCCTTCGAGTACCGCACCGACTGGGACCGGTCCGAG
GACGACGCGAAGAACCTCGCCTGGATCAAGGCGCTGCGCCACGCCATGGCCCCGCACACCACCGGCGCCGCGTACGTCAACACCATC
GACCTGGCCCTGGAGAACTGGCTCTGGGCCTACTACGAGGAGAACCTGCCGCGGCTGATCGAGGTCAAGCGCCGCTACGACCCCGGC
GACGTCTTCCACCACCCGCACAGCATCCCGCTCACGGTCACCGAGGCGGACATCGCCCGGTACGGCATACCGGAAGCGGTGGCCGCA
CGGCTGCGGGCGGCAGGGCTGACGGACCGTCAGTAG
SEQ ID NO: 6-OLS
MNHLRAEGPASVLAIGTANPENILLQDEFPDYYFRVTKSEHMTQLKEKFRKICDKSMIRKRNCFLNEEHLKQNPRLVEHEMQTLDAR
QDMLVVEVPKLGKDACAKAIKEWGQPKSKITHLIFTSASTTDMPGADYHCAKLLGLSPSVKRVMMYQLGCYGGGTVLRIAKDIAENN
KGARVLAVCCDIMACLFRGPSESDLELLVGQAIFGDGAAAVIVGAEPDESVGERPIFELVSTGQTILPNSEGTIGGHIREAGLIFDL
HKDVPMLISNNIEKCLIEAFTPIGISDWNSIFWITHPGGKAILDKVEEKLHLKSDKFVDSRHVLSEHGNMSSSTVLFVMDELRKRSL
EEGKSTTGDGFEWGVLFGFGPGLTVERVVVRSVPIKY
SEQ ID NO: 7-OAC
MAVKHLIVLKFKDEITEAQKEEFFKTYVNLVNIIPAMKDVYWGKDVTQKNKEEGYTHIVEVTFESVETIQDYIIHPAHVGFGDVYRS
FWEKLLIFDYTPRK
SEQ ID NO: 8-Prenyltransferase
MSGAADVERVYAAMEEAAGLLGVTCAREKIYPLLTEFQDTLTDGVVVFSMASGRRSTELDFSISVPTSQGDPYATVVDKGLFPATGH
PVDDLLADTQKHLPVSMFAIDGEVTGGFKKTYAFFPTDDMPGVAQLSAIPSMPSSVAENAELFARYGLDKVQMTSMDYKKRQVNLYF
SELSEQTLAPESVLALVRELGLHVPTELGLEFCKRSFSVYPTLNWDTGKIDRLCFAVISTDPTLVPSTDERDIEQFRHYGTKAPYAY
VGENRTLVYGLTLSPTEEYYKLGAYYHITDIQRRLLKAFDALED
SEQ ID NO: 9-Polynucleotide encoding SEQ ID NO: 1 (Tcz9)
ATGGCCACCCCATCCGCATTCTCCGGCAGCGTCCTCACCCCCGGTGACGACGGCTTCGAGGCGGCCCAGGTCACCTGGAACGCCTGC
TACTCCTCCCGCCCCCGGGAGGTGATGGTCTGCCACGACGCCGCGTCCGTCGCCGAGGCGGTGCGCAGCGTGCGGGAGCGCGGACTG
CCCTTCCGGGTGCGCTCCGGCGGCCACTCGATGTGCGGGCTGAGCAACCTGGACGACGGCGTCATCATCGACCTCGGCGGCCTGGGC
GGCGTCGAACTCACCCCGGACCGGCAGACGGTGCGGATCGGCGGCGGCGCCCGCCTCGCCGACGTCTACAACACCCTCTGGGACCAC
CGGCTGACCGTCCCGGCCGGCACCTGCCCGCGGATCGGGGTCGGCGGCCACGTGCTGGGCGGCGGCATGGGCGTGCTGTCCCGGTCG
CGTGGCGCGCTGGTGGACCATCTGACGGCACTGGAGATGGTGGACGCCGAGGGCCGGCTCCTGCGCGTCAGCGAGGACGAGAACCCC
GACCTGTTCTGGGCCTGCCGGGGCGGCGGTGGCGGCAACTTCGGCATCGTGACCGCGTACGAGCTGCGGCCCACGCCGATCGACGAC
GTCACCATCTTCACGGTCAGCTGGACCTGGTCGCAGCTCCCCGACGCGGTGCGGGCCTGGCAGCGGTGGCTGGGCTCGGCGGAGAGC
CGTATCAACAGCTTCCTCTCACTCTTCCCCCAACAGCAGGACATGGTCGTCGCGTTCGGCGTCTTCGACGGTCCGGCCGCCGACTTC
CGGCCGCTGCTGGCCCCGCTGACCGCCGAGGTCGCCCCGGAGGCCGAGGTCGTGGAGGAGGTCCCGTTCATCCAGGCCGTGGACACC
GTCGAGGCGTTGCAGGGCGAGGCCGCCGCCGCCGAGCAGGTGCGGGCGCAGGGCAGTTCGGCGATCATCGCGAACCCGCTGAACGAC
GAGGCGCTGGCCACGCTCCAGGAGTTCCTCACCGACCCGCCCAGCCACCGAGCGGAGGTGGCCGTCTACGGGATGGGCGGCGTCATC
GGCGAACGGGAGCGCGGCGACACGGCGTTCGTGCACCGCACCGGCCTGATGGCCTTCGAGTACCGCACCGACTGGGACAGCCCCGAG
GACGACCGGCTCAACCTGGACTGGGTGACCCGGCTGCGGCACGCGATGGCCGAACACACCACCGGCGCCGCCTACGTCAACACCATC
GACCTGGCCCTGGAGAACTGGCTGTGGGCCTACTACGAGGAGAACCTGCCGCGCCTGATGGCGGTCAAGCGGCGGTACGACCCGGAG
AACGTCTTCCACCACCCGCACAGCATCCCCGGCTCGCTCACCGCCGAGGCGGCCCGGGCGCACGGCGTTCCCGAGGCCACCCTCAAG
CGGCTGCACGACGACGGCCTGCTCGACGGACCGCTGGACTGA
SEQ ID NO: 10-Accession ID No. WP_235454663.1 from Streptomyces
olivochromogenes (″Sox9″)
MGTPARFTGTVLTPGDAGFEDASRTWNACYASRPREVMVCHSAEAVAEALGSARGRGIPFRVRSGGHSMAGLSSVADGAIIDLGGLD
AITLSEDSSLVTVGGGARLGDIYRELWKAGVTVPAGTCPRIGIGGHVLGGGMGVLSRSRGALVDHLVGLELVGADGRLRQVDDDEHA
DLFWACRGGGGGSFGIATSYTFRTSPISEVTIFTIAWSWAQFAPAVKAWQGWLASAEDRINTFLSLFPRQQDLLVAFGVFDGPAAEF
EELLRPLVAAAEPQTLVVEPVPFIQAVDTVEALQGEAAAADQVRAQGSSAIVAEPLNEAAIATLQEFLTDPPSHRAEVAVYGMGGAI
GHRARTDTAFVHRTGLMAFEYRTDWDLPEDDGKNLAWVRALRHSMAEHTTGAAYVNTIDLALENWLWAYYEENLPRLMQVKRQYDPG
DVFQHPHSIPVSLTAEDIARHGIPEQVAAEIRSAGLLS
SEQ ID NO: 11-Accession ID No. ANW12117.1 from Streptomyces albogriseolus
(″ThdO″)
MDRYPRNAGTVVSPADPDYERARRIWNNRFDCRPRAIMYPRDEAEAAAALGYARDNEVPFRIRSGGHNSEGFCTVDDGIIIDLSKLG
SIAVNNDRTRAVISPGVMLGEVYANLWSVGGTIPAGVCPDIRIGGHVLGGGIGMLVRSRGLLIDNLVGLTVVDARGERLQVDADHHP
DLFWACKGGGGGNYGIATSYTFEMRPISDVTIFKLQWDWQGGMAVLDAWQHWLLSADSRVNARFNVFSSTVGTVMTVGLFEGSADEL
KSILQPLVEKFPPRDSFVRTMPYMECVGTFSEQVPSIRAKFVPALSAGPLDADALKTLERWHKDAPTGVKTGLYGLGGEVLGSVSPD
SSAFAHRDASICVEYLGHWQGAEHDREHLGWLSGVREEMDAFMTGGAYVNSPDRDLENWLHAYYGESLPRLMDVKRRYDPDNVFAFE
QSIPGSLTLPEARAAGLTDTVVEDLRARGLLSA
SEQ ID NO: 12-Accession ID No. WP_067055472.1 from Streptomyces sp. FXJ1.172
(″ThnG″)
MGAYPDTIGTVVTPGDTRYERARRLWNERFDYRPRAVVLPGDSHETAAALRYVRERGIPFRIRSGGHNSEGFSGLDDGIVIDVSRLG
ALAVYGDRTRATLGPGVLLGEAYAALWALGGTIPAGVCPDIRIGGHVLGGGIGMLVRSRGLLIDHLVALTLIDARGEELTVDADHHP
DLLWASRGGGGGNFGIVTSYTFAMRPIGDVTLFTLEWDARDGIAALDAWQHWLSQADPRVNSRENLFPASADRALATGLFEGGEREL
TALLRPLTNACRPRATSVRALPYGDSIGTWTEQVPSVRAKFTPALAEGPLGAEALRTLGRRHEKAPPGVKTGLYGLGGAVLADVPPD
HSAFAHRAAFLCVEYLGHWSDPGSDAEHLDWLSGTRAEMLPFMTGGAYVNSPDLDLENWLHAYYGDGLPRLMEVKRRYDPHGLFTFE
QSIPTALTRAQARAARLPGPVVADLHARGLLTD
SEQ ID NO: 13-Accession ID No. WP_098755161.1 from Streptomyces sp. Ru87
(″SrThX″)
MEEHPQHVGTVVTQADPDYEQARHIWNERFDFRPRAVFYPRNAEETAAAVTHVRDRGLPFRIRSGGHNSEGFCSVDDGVVIDLSKLA
SIGVNNDRTRATTGPGVLLGEVYATLAAVGGTIPAGVCPDIRIGGHVLGGGIGMLVRSRGLLIDSLVGLTLVDARGRILEVDDSSHP
ELMWACRGGGGGNYGIVTSYTFEMRPVSEVTLFTLEWDWNGGIGVLDTWQHWILRADTRVNARFAFLPGSTGTVMSTGLFEGPAREL
ESILRPLVGKHPPRDSVVRTVPYAESVGTFTEPVPSIRAKFVPALAGGPLGTEALEILERRHRDAPAGVKTGLYGLGGAVLDNAPAK
RSSFAHRDAPICVEYLGHWKDSAHDSEHLDWLTATREELHGFMTGGAYVNSPDRDLADWLHAYYGENLPGLMDVKRRYDPDDVFSFE
QSIPTSLSPAEARAAGLTDTVVAELRGRGLLRS
SEQ ID NO: 14-Polynucleotide encoding SEQ ID NO: 11 (ThdO)
ATGGACAGGTATCCCCGGAACGCCGGCACGGTGGTCTCGCCGGCCGACCCGGACTACGAACGGGCACGACGCATCTGGAACAACCGC
TTCGACTGCCGGCCCCGCGCCATCATGTACCCGCGCGACGAAGCGGAGGCGGCGGCCGCGCTGGGATACGCGCGGGACAACGAGGTC
CCGTTCCGTATCCGCTCCGGCGGTCACAACAGCGAGGGTTTCTGCACGGTCGACGACGGCATCATCATCGACCTGTCGAAGCTCGGC
TCGATCGCCGTGAACAACGACCGGACCCGGGCCGTCATCAGCCCCGGTGTCATGCTCGGTGAGGTGTACGCGAACCTCTGGTCCGTG
GGCGGCACCATCCCCGCCGGGGTCTGCCCGGACATCAGGATCGGCGGGCACGTGCTCGGCGGCGGCATCGGGATGCTCGTCCGCTCG
CGGGGCCTGCTCATCGACAATCTGGTGGGCCTCACCGTGGTCGACGCGCGGGGGGAGAGGCTCCAGGTCGACGCGGACCACCACCCG
GATCTCTTCTGGGCCTGCAAGGGAGGCGGCGGCGGCAACTACGGGATCGCCACCTCGTACACCTTCGAGATGCGGCCCATCTCCGAC
GTGACGATCTTCAAGCTGCAGTGGGACTGGCAGGGCGGCATGGCGGTGCTCGACGCCTGGCAGCACTGGCTCCTCTCGGCGGACTCG
CGGGTCAACGCCCGGTTCAACGTCTTCTCCAGTACGGTCGGTACGGTCATGACGGTCGGCCTGTTCGAGGGTTCGGCCGACGAGCTG
AAGTCGATCCTCCAGCCGCTGGTCGAGAAGTTCCCGCCGCGTGACTCCTTCGTACGCACCATGCCCTACATGGAGTGCGTGGGGACG
TTCAGCGAGCAAGTGCCCTCCATACGGGCCAAGTTCGTGCCCGCGCTGTCGGCGGGACCGCTCGACGCCGACGCACTGAAGACCCTG
GAGCGCTGGCACAAGGACGCACCGACCGGGGTCAAGACGGGGCTCTACGGGCTCGGCGGCGAGGTGCTCGGCAGTGTGTCGCCGGAC
AGCTCGGCGTTCGCCCACCGCGACGCGTCCATCTGCGTCGAGTACCTCGGCCACTGGCAGGGGGCGGAACACGACCGGGAACACCTC
GGCTGGCTGTCCGGCGTCCGCGAGGAGATGGACGCCTTCATGACGGGCGGAGCCTACGTCAACAGTCCGGACCGCGATCTGGAGAAC
TGGCTGCACGCCTACTACGGGGAGAGCCTCCCGCGGCTGATGGACGTGAAGAGGCGGTACGACCCCGACAACGTCTTCGCCTTCGAG
CAGAGCATCCCGGGATCCCTCACCCTCCCGGAGGCCCGCGCCGCGGGCCTGACCGACACGGTCGTCGAAGACCTGCGCGCCCGCGGA
CTTCTCTCCGCCTGA
SEQ ID NO: 15-Polynucleotide encoding SEQ ID NO: 12 (ThnG)
ATGGGGGCGTATCCGGACACCATCGGCACGGTGGTGACACCCGGCGACACGCGCTACGAAAGGGCGCGCCGCCTGTGGAACGAGCGC
TTCGACTACCGGCCCAGGGCCGTCGTCCTGCCGGGTGACAGCCACGAGACGGCAGCGGCGCTCCGCTACGTCCGGGAGCGCGGCATC
CCGTTCCGGATCCGCTCCGGCGGCCACAACAGCGAGGGGTTCAGCGGCCTCGACGACGGCATCGTCATCGACGTGTCGCGCCTGGGG
GCGCTGGCGGTCTACGGTGACCGCACCCGCGCCACCCTCGGCCCGGGTGTCCTGCTCGGCGAGGCGTACGCGGCGCTGTGGGCGCTC
GGCGGCACGATCCCCGCGGGCGTCTGCCCCGACATCCGCATCGGCGGTCATGTCCTGGGCGGCGGCATCGGCATGCTGGTACGTTCC
CGCGGCTTGCTCATCGACCACCTGGTCGCACTCACCCTGATCGACGCCCGCGGCGAGGAACTGACGGTCGACGCGGACCACCATCCG
GACCTGCTCTGGGCGTCACGCGGAGGCGGTGGCGGGAACTTCGGGATCGTCACCTCCTACACCTTCGCGATGCGCCCGATCGGCGAC
GTGACCCTGTTCACGCTGGAATGGGACGCGCGCGACGGCATCGCCGCACTCGACGCCTGGCAGCACTGGCTCAGCCAGGCGGACCCG
CGGGTGAACTCCCGCTTCAACCTGTTCCCCGCCTCCGCCGACAGGGCCCTGGCCACCGGCCTGTTCGAAGGGGGTGAGCGTGAACTC
ACCGCCCTGCTCCGACCGTTGACCAACGCCTGCCGGCCTCGCGCGACATCCGTACGCGCCCTGCCGTACGGGGACAGCATCGGCACC
TGGACCGAGCAAGTGCCGTCGGTACGCGCCAAGTTCACCCCCGCACTGGCCGAAGGCCCACTCGGCGCCGAGGCGCTGCGCACCCTG
GGCCGCCGGCACGAGAAGGCGCCGCCCGGCGTCAAGACGGGTCTGTACGGCCTGGGCGGAGCGGTCCTGGCCGACGTGCCGCCCGAT
CACTCCGCGTTCGCGCACCGGGCGGCCTTCCTGTGCGTGGAGTACCTCGGCCACTGGAGCGATCCCGGCAGCGACGCCGAACACCTG
GACTGGCTGTCCGGCACCCGCGCGGAGATGCTCCCGTTCATGACCGGCGGGGCCTACGTCAACAGCCCGGACCTCGATCTGGAGAAC
TGGCTGCACGCCTACTACGGCGACGGCCTGCCGCGCCTCATGGAGGTCAAGCGCCGCTACGACCCGCACGGCCTCTTCACGTTCGAG
CAGAGCATTCCCACCGCGCTCACCCGGGCCCAGGCCCGTGCCGCCCGGCTCCCCGGCCCGGTCGTCGCCGACCTGCACGCCCGCGGC
CTGCTCACCGACTGA
SEQ ID NO: 16-Polynucleotide encoding SEQ ID NO: 13 (SrThX)
ATGGAGGAACACCCGCAGCACGTCGGCACTGTGGTGACACAGGCCGATCCGGACTACGAGCAGGCACGGCACATCTGGAACGAGCGT
TTCGACTTCCGGCCCCGGGCGGTGTTCTACCCCCGGAACGCCGAGGAGACGGCGGCCGCCGTCACGCATGTACGGGACCGGGGGCTG
CCGTTCCGCATCAGGTCCGGCGGTCACAACAGCGAGGGCTTCTGCAGTGTGGACGACGGCGTCGTCATCGATCTGTCGAAGCTGGCG
TCGATAGGCGTCAACAACGACCGGACCCGGGCGACGACCGGCCCCGGTGTCCTGCTCGGTGAGGTCTACGCGACTCTGGCCGCCGTC
GGCGGCACCATCCCGGCCGGCGTCTGCCCCGACATCCGGATCGGCGGGCACGTGCTCGGCGGCGGCATCGGGATGCTCGTGCGCTCC
CGCGGCCTGCTCATCGACAGCCTGGTGGGGCTCACCCTGGTCGACGCCCGGGGCCGGATCCTGGAGGTCGACGACTCCTCGCACCCG
GAGCTGATGTGGGCCTGCCGTGGCGGCGGCGGCGGGAACTACGGAATCGTCACGTCGTACACCTTCGAGATGCGGCCGGTCTCCGAG
GTGACGCTCTTCACGCTGGAGTGGGACTGGAACGGCGGCATCGGCGTCCTCGACACCTGGCAGCACTGGATCCTCCGGGCGGACACG
CGCGTGAACGCCCGGTTCGCGTTCCTGCCCGGCTCCACCGGAACGGTCATGTCCACCGGGCTGTTCGAGGGGCCGGCGCGGGAGCTG
GAGTCGATCCTCCGGCCGCTGGTCGGAAAGCACCCGCCGCGCGATTCCGTCGTCCGCACGGTGCCGTACGCGGAGAGCGTGGGCACG
TTCACCGAACCGGTCCCCTCCATCCGGGCCAAGTTCGTGCCCGCGCTCGCCGGCGGCCCGCTCGGCACCGAGGCCCTGGAGATCCTG
GAACGCCGGCACCGGGACGCGCCGGCCGGCGTCAAGACGGGGCTGTACGGACTCGGCGGCGCGGTGCTCGACAACGCGCCCGCGAAG
CGCTCGTCGTTCGCGCACCGGGACGCGCCCATCTGCGTGGAGTACCTCGGGCACTGGAAGGACTCCGCGCACGACAGCGAGCACCTC
GACTGGCTGACCGCGACCCGCGAGGAGCTGCACGGCTTCATGACCGGTGGCGCCTACGTCAACAGCCCGGACCGCGACCTCGCCGAC
TGGCTCCACGCCTACTACGGCGAGAACCTGCCCGGACTCATGGACGTCAAGCGCCGTTACGACCCGGACGACGTGTTCTCGTTCGAG
CAGAGCATCCCCACCTCCCTCTCCCCCGCCGAGGCCCGCGCCGCGGGACTGACCGACACGGTCGTCGCGGAACTGCGCGGGCGCGGC
CTGCTCCGCTCCTGA
SEQ ID NO: 17 Accession ID No. WP_156205671.1 Streptomyces lycii (″Slx9″)
MEEHPQHVGTVVTQADPDYEQARHIWNERFDERPRAVFYPRNAEETAAAVTHVRDRGLPFRIRSGGHNSEGFCSVDDGVVIDLSKLA
SIGVNNDRTRATTGPGVLLGEVYATLAAVGGTIPAGVCPDIRIGGHVLGGGIGMLVRSRGLLIDSLVGLTLVDARGRILEVDDSSHP
ELMWACRGGGGGNYGIVTSYTFEMRPVSEVTLFTLEWDWNGGIGVLDTWQHWILRADTRVNARFAFLPGSTGTVMSTGLFEGPAREL
ESILRPLVGKHPPRDSVVRTVPYAESVGTFTEPVPSIRAKFVPALAGGPLGTEALEILERRHRDAPAGVKTGLYGLGGAVLDNAPAK
RSSFAHRDAPICVEYLGHWKDSAHDSEHLDWLTATREELHGFMTGGAYVNSPDRDLADWLHAYYGGNLPGLMDVKRRYDPDDVFSFE
QSIPTSLSPAEARAAGLTDTVVAELRGRGLLRS
SEQ ID NO: 18-Accession ID No. WP_239335163.1 Frankia sp. CiP3 (″Frx9″)
MGTYPDTIGLVVTPSDTRYDRARSIWNERFDCRPQAVVYPRDAEETAAALCYVRDHGISFRIRSGGHNVEGFCTLDDGIVIDVSRLG
SVAVNNDRSRAVIGPGVLLGEVYAVLWSVGGTIPAGVCPDIRIGGHVLGGGIGMLVRSRGLLIDSLVGLTVVDARGRILAVDETNHA
DLLWASRGGGGGNFGIVTSYTFKMRPISDVTLFTMKWDWADGIAVLDEWQRWILRADERVNVRENLFPRSAGMAMTTGLFEGSADEL
KSIIKPLVDTHPPRETSMRTVPYMDSVGTWTQRVPSIRAKFVPALAARPLDSYAMKILDRWHRTAPAGVRTGFYGLGGAVLGDVPPE
HSAFAHRNASICVEYLGHWDDAESDREHLDWLCGIRDEMLPFMTGGAYVNSPDRDLANWLHAYYGDRLPRLMDVKRRYDPHNVFSFE
QSIPTSLTLPEAHAAGLPDAVIAELRSRNLLSEHA
SEQ ID NO: 19-Accession ID No. WP_211768552.1 Kutzneria sp. CA-103260 (″Kcx9″)
MSRSQRVAATVTAADPGYDQARRIWNERFDCRPLAVLYPADVAEVAGAVRAARDDGVPFRIRSGGHNVEGFSVLDDGYVIDLSRLTG
VTVDDDRTRGTVAAGTLLGDVYSGLWAAGVTIPAGVCPDIRIGGHVLGGGVGMLVRSRGMLVDSLVGLTMVDADGEVLEVNDDTHPD
LMWACRGGGGGNFGVATSYTFHTRPIGDVTLVTAAWNEDNAITALDRWQHWAVEVDQRVNARFSLFSPGTGGGRMAVLFEGTSAEWE
SLARPLFEQCPPAEHTVRETAYINSVGQFSQRVPSIRAKFVPALAAGPLDAEALKILERHHRAAPDGVKTSFYGLGGAVLGADLAKR
SAFAHRDALFCVEYLGHWHGAEHDAEQLGWLAGIRDELRPFMTGGAYVNSPDRDLTDWLHAYYGASLPRLMEVKRRYDPEDVFRFEQ
SIPVSLSPAAARAAGLTEAVVADLGARGLLTS
SEQ ID NO: 20-Polynucleotide encoding SEQ ID NO: 10 (Sox9)
ATGGGAACACCTGCAAGATTTACGGGTACAGTTTTAACGCCGGGGGACGCGGGCTTTGAAGATGCATCGCGGACTTGGAACGCTTGT
TACGCGTCGCGGCCCCGTGAAGTCATGGTTTGCCACTCGGCGGAGGCGGTGGCCGAGGCGCTTGGTTCTGCTCGTGGTCGTGGTATT
CCTTTCCGTGTGCGGTCCGGGGGCCACTCCATGGCGGGGCTTTCCTCCGTGGCAGACGGAGCGATCATCGACTTGGGCGGGCTTGAT
GCCATTACCCTGAGCGAAGATAGCAGCCTCGTGACGGTCGGGGGGGGCGCGCGCTTGGGTGATATTTATCGTGAACTCTGGAAGGCT
GGTGTGACGGTGCCGGCCGGTACTTGTCCCCGTATCGGCATCGGCGGCCACGTGTTGGGCGGCGGAATGGGCGTACTAAGTCGTAGC
CGTGGTGCGCTGGTGGATCATCTGGTGGGACTGGAACTGGTGGGCGCGGACGGCCGCCTGCGCCAAGTGGACGACGACGAACATGCA
GATCTGTTTTGGGCTTGCCGCGGCGGCGGGGGAGGCAGCTTCGGAATCGCGACGAGTTACACGTTCCGCACGTCACCGATTAGCGAA
GTTACCATTTTTACAATTGCTTGGTCTTGGGCACAGTTTGCACCGGCTGTTAAAGCATGGCAGGGTTGGCTCGCGTCAGCGGAGGAC
AGAATAAACACCTTCCTTTCTTTATTCCCTAGGCAGCAGGATTTGCTGGTGGCGTTTGGTGTTTTTGATGGTCCGGCGGCCGAATTC
GAAGAATTACTCCGTCCGCTGGTCGCGGCAGCGGAGCCGCAAACGCTGGTAGTTGAACCCGTGCCTTTTATTCAGGCGGTCGATACT
GTGGAGGCGCTGCAGGGCGAGGCGGCGGCAGCCGATCAAGTACGCGCGCAAGGTTCATCAGCGATCGTCGCCGAGCCGCTGAACGAA
GCCGCAATCGCCACCCTGCAGGAATTCTTGACCGATCCGCCAAGTCACCGCGCCGAAGTCGCCGTTTACGGCATGGGGGGGGCCATT
GGCCATCGCGCCCGCACCGATACCGCATTTGTTCATCGCACCGGTTTAATGGCCTTTGAATATCGCACAGATTGGGATTTACCAGAG
GACGACGGTAAAAATCTGGCGTGGGTCCGCGCACTGCGACATAGTATGGCCGAACATACCACCGGTGCTGCTTACGTTAATACCATT
GATCTGGCACTGGAGAACTGGTTGTGGGCTTATTATGAAGAAAATCTGCCGCGTCTAATGCAAGTAAAACGTCAGTATGATCCGGGC
GATGTTTTCCAGCATCCGCACTCGATCCCAGTAAGCCTGACCGCCGAAGATATTGCTCGTCACGGGATCCCAGAACAGGTCGCCGCC
GAAATTCGCAGCGCCGGTCTGCTGTCTTAG
SEQ ID NO: 21-Polynucleotide encoding SEQ ID NO: 17 (Slx9)
ATGGAAGAACACCCACAACACGTAGGAACAGTGGTGACGCAAGCGGACCCGGACTATGAACAAGCACGTCATATTTGGAACGAACGC
TTTGATTTTCGTCCGCGGGCGGTGTTCTATCCTCGTAATGCGGAAGAAACGGCGGCGGCCGTGACGCATGTTCGTGACCGGGGGCTT
CCTTTTCGTATTCGCTCGGGCGGGCACAATAGCGAGGGTTTCTGCTCTGTTGATGACGGTGTGGTAATCGATTTGTCGAAGTTAGCA
TCGATCGGCGTGAATAACGACCGTACCCGTGCTACTACTGGTCCGGGGGTGTTGCTCGGTGAGGTGTACGCCACGTTGGCGGCGGTG
GGTGGTACCATCCCGGCCGGCGTGTGTCCTGATATTCGCATCGGCGGCCACGTGTTGGGAGGGGGCATCGGGATGCTGGTTCGTTCG
CGCGGGTTGCTGATTGATTCCCTGGTGGGGCTGACGCTGGTGGATGCTCGCGGCCGCATTCTGGAGGTGGACGATAGTAGCCATCCC
GAACTAATGTGGGCATGTCGCGGCGGAGGAGGCGGCAACTATGGTATTGTTACCTCTTATACTTTTGAAATGCGTCCAGTTAGCGAA
GTAACACTGTTCACTTTAGAATGGGACTGGAACGGCGGAATCGGCGTCCTGGACACTTGGCAGCACTGGATCCTTCGTGCCGATACA
CGCGTTAATGCTAGATTTGCCTTCCTTCCTGGTTCCACGGGCACGGTCATGTCCACGGGACTGTTCGAAGGTCCAGCACGCGAGTTG
GAATCGATATTACGCCCGCTGGTCGGCAAACACCCGCCGCGGGATAGTGTAGTTAGGACCGTCCCGTACGCTGAATCCGTCGGCACC
TTTACCGAACCGGTCCCGTCAATTCGTGCCAAATTCGTTCCGGCGCTGGCGGGCGGCCCGCTCGGTACAGAAGCCCTGGAGATTCTG
GAACGTCGCCACCGCGACGCGCCGGCAGGCGTCAAAACCGGCCTGTATGGCCTGGGCGGAGCGGTCCTCGACAACGCGCCCGCGAAA
CGTAGTTCATTCGCGCATCGCGACGCCCCGATTTGCGTCGAATATTTAGGTCATTGGAAGGATAGCGCCCATGATTCTGAACATTTA
GATTGGCTGACCGCAACCCGGGAGGAGCTTCACGGTTTTATGACCGGTGGCGCGTACGTGAATAGCCCTGATCGTGATCTGGCTGAT
TGGCTCCATGCATATTACGGTGGTAACCTGCCGGGTCTGATGGATGTTAAACGTCGATACGATCCAGATGATGTTTTTAGTTTTGAA
CAGTCAATCCCAACCTCTCTATCACCCGCAGAGGCCCGCGCAGCCGGTCTGACCGATACCGTAGTAGCTGAGTTACGCGGTCGCGGT
CTGCTGCGAAGCTAG
SEQ ID NO: 22-Polynucleotide encoding SEQ ID NO: 18 (Frx9)
ATGGGAACATACCCTGATACAATAGGTCTGGTTGTTACCCCGAGCGATACGCGGTACGACCGGGCACGGTCGATCTGGAATGAACGT
TTTGATTGTCGTCCTCAGGCAGTCGTGTATCCTCGGGACGCGGAGGAAACGGCTGCTGCCTTATGTTATGTCCGTGATCATGGGATC
AGTTTTCGCATTCGTTCGGGCGGGCACAATGTGGAGGGGTTCTGCACGCTTGACGACGGGATCGTAATTGATGTTTCTCGCTTAGGT
AGCGTGGCCGTCAATAATGACCGCTCCCGCGCGGTGATCGGTCCGGGGGTGTTGCTGGGTGAGGTGTACGCGGTGCTTTGGTCCGTG
GGCGGTACCATCCCGGCCGGAGTGTGCCCGGATATTCGTATCGGCGGCCATGTGTTGGGCGGGGGCATTGGTATGCTGGTGCGCTCC
CGAGGCCTTCTTATCGATAGCCTGGTTGGTCTGACCGTCGTTGATGCCCGTGGTCGTATTCTGGCCGTTGATGAGACAAACCACGCT
GACTTGTTGTGGGCGTCCCGCGGCGGCGGAGGCGGCAATTTCGGAATCGTGACTAGCTATACATTTAAGATGCGTCCCATTAGCGAT
GTTACCCTGTTCACTATGAAATGGGATTGGGCGGACGGAATCGCGGTGTTGGACGAATGGCAACGCTGGATTTTACGTGCAGATGAA
CGAGTTAATGTTAGGTTCAACCTGTTTCCACGTAGCGCCGGTATGGCAATGACGACCGGCCTGTTCGAAGGCTCAGCCGATGAACTC
AAGAGTATTATTAAACCGCTGGTAGATACCCACCCGCCGCGCGAGACATCGATGCGTACCGTCCCCTATATGGATTCAGTGGGCACC
TGGACCCAGCGTGTCCCATCTATTCGTGCGAAATTTGTACCCGCCTTAGCTGCTCGCCCACTGGATTCTTATGCAATGAAAATTTTA
GATCGCTGGCATCGTACTGCGCCGGCAGGCGTCCGCACGGGCTTCTACGGCCTAGGAGGCGCGGTACTGGGGGATGTTCCGCCAGAA
CACTCGGCGTTTGCACATCGCAACGCGTCAATCTGTGTCGAATATTTAGGTCATTGGGACGACGCGGAAAGTGACCGCGAACATCTG
GATTGGCTGTGCGGTATTCGTGATGAAATGCTCCCTTTTATGACCGGTGGCGCGTATGTCAACTCTCCGGACAGAGATCTAGCTAAC
TGGCTGCATGCCTACTATGGTGATCGTCTGCCCAGACTCATGGATGTGAAACGCCGCTACGACCCGCACAACGTATTTAGTTTTGAA
CAGTCGATACCAACCTCACTGACGCTGCCGGAGGCGCACGCAGCAGGTCTGCCGGACGCGGTAATCGCCGAACTCCGCAGTCGCAAC
CTGCTGAGCGAACATGCCTAG
SEQ ID NO: 23-Polynucleotide encoding SEQ ID NO: 19 (Kcx9)
ATGAGTAGGAGTCAAAGAGTGGCCGCAACTGTGACGGCGGCAGATCCTGGATATGATCAAGCTCGTCGTATTTGGAACGAACGTTTT
GATTGCCGGCCGCTCGCGGTGCTTTACCCGGCGGACGTGGCGGAAGTAGCGGGAGCGGTCAGAGCAGCTCGGGACGACGGGGTCCCG
TTTCGTATTCGTTCGGGCGGGCACAATGTTGAAGGTTTTAGCGTTCTCGACGACGGCTACGTTATTGACTTATCGCGCCTTACGGGG
GTCACGGTTGATGATGATCGTACGCGCGGGACGGTCGCGGCGGGGACGTTACTGGGGGACGTCTATTCTGGTCTGTGGGCGGCGGGC
GTGACCATCCCGGCGGGGGTGTGCCCTGATATACGTATCGGCGGCCACGTGTTGGGCGGAGGCGTGGGCATGCTTGTGCGTTCGCGC
GGCATGTTGGTGGATTCTTTAGTGGGCCTTACCATGGTGGACGCGGACGGCGAGGTGTTGGAGGTGAACGACGATACACATCCTGAT
CTGATGTGGGCTTGCCGCGGAGGAGGCGGCGGCAATTTTGGTGTTGCTACTAGCTATACATTTCACACGCGGCCGATTGGCGACGTG
ACCCTAGTGACCGCGGCGTGGAACTTTGATAATGCTATTACCGCGTTGGATCGTTGGCAGCATTGGGCTGTTGAAGTAGACCAGCGC
GTCAATGCACGTTTCTCCTTGTTCTCCCCTGGTACCGGGGGCGGCCGTATGGCGGTCCTGTTCGAGGGTACTTCAGCCGAATGGGAA
AGCCTGGCGCGCCCGCTATTCGAGCAGTGTCCGCCCGCGGAGCACACCGTCCGAGAAACTGCTTATATTAACAGCGTTGGTCAATTC
TCCCAGCGAGTCCCGTCAATCCGTGCAAAATTTGTTCCGGCGCTGGCGGCAGGCCCGCTGGACGCCGAGGCTTTAAAAATTCTGGAA
CGTCATCACCGCGCCGCCCCGGATGGCGTAAAGACCTCATTTTACGGCCTGGGAGGCGCCGTACTGGGTGCCGATCTGGCCAAACGT
AGCGCTTTCGCACATCGAGATGCCCTGTTCTGTGTTGAATATCTCGGTCATTGGCATGGTGCCGAGCACGATGCCGAACAACTGGGT
TGGCTGGCAGGTATCCGCGATGAACTGCGCCCATTTATGACCGGTGGTGCCTATGTTAACTCGCCGGATCGGGATCTGACCGATTGG
TTGCATGCATACTATGGTGCTAGCCTGCCCCGCCTGATGGAAGTAAAACGCCGCTACGATCCAGAAGATGTTTTTCGCTTCGAACAG
AGTATCCCAGTAAGTCTGAGCCCAGCCGCCGCACGCGCCGCAGGTCTGACAGAAGCAGTCGTAGCCGATCTCGGTGCCCGCGGTTTA
CTGACCTCTTAG

1. A non-natural flavin-dependent oxidase comprising at least one amino acid variation as compared to a wild type flavin-dependent oxidase, wherein the non-natural flavin-dependent oxidase: (i) does not comprise a disulfide bond (ii) is capable of oxidative cyclization of a prenylated aromatic compound into a cannabinoid, and (iii) comprises at least 70% sequence identity to any one of SEQ ID NOs:1-3, any one of SEQ ID NOs:10-13, or any one of SEQ ID NOs:17-19.

2. The non-natural flavin-dependent oxidase of claim 1, wherein the prenylated aromatic compound is cannabigerolic acid (CBGA), cannabigerorcinic acid (CBGOA), cannabigerivarinic acid (CBGVA), cannabigerorcinol (CBGO), cannabigerivarinol (CBGV), or cannabigerol (CBG).

3. The non-natural flavin-dependent oxidase of claim 1, wherein the non-natural flavin-dependent oxidase is not glycosylated.

4. The non-natural flavin-dependent oxidase of claim 1, wherein the non-natural flavin-dependent oxidase comprises a monovalently bound FAD cofactor, or wherein the non-natural flavin-dependent oxidase comprises a bivalently bound FAD cofactor.

5. The non-natural flavin-dependent oxidase of claim 1, wherein the non-natural flavin-dependent oxidase is capable of oxidative cyclization of a prenylated aromatic compound into a cannabinoid at about pH 7.5.

6. The non-natural flavin-dependent oxidase of claim 1, wherein catalytic activity of the non-natural flavin-dependent oxidase is substantially the same from about pH 5 to about pH 8.

7. The non-natural flavin-dependent oxidase of claim 1, comprising at least 80% sequence identity to any one of SEQ ID NOs:1-3, any one of SEQ ID NOs:10-13, or any one of SEQ ID NOs:17-19, optionally comprising at least 90% sequence identity to any one of SEQ ID NOs:1-3, any one of SEQ ID NOs:10-13, or any one of SEQ ID NOs:17-19.

8. The non-natural flavin-dependent oxidase of claim 7, comprising at least 90% sequence identity to any one of SEQ ID NOs:1-3, SEQ ID NO:10, SEQ ID NO:11, SEQ ID NO:13, or SEQ ID NO:19.

9. The non-natural flavin-dependent oxidase of claim 6, wherein the non-natural flavin-dependent oxidase converts CBGA to cannabichromenic acid (CBCA), cannabidiolic acid (CBDA), and/or Δ9-tetrahydrocannabinolic acid (THCA), optionally wherein the converting is at about pH 4 to about pH 9.

10. The non-natural flavin-dependent oxidase of claim 1, further comprising an affinity tag, a purification tag, a solubility tag, or a combination thereof.

11. A polynucleotide comprising a nucleic acid sequence encoding the non-natural flavin-dependent oxidase of claim 1.

12. A polynucleotide comprising: (a) a nucleic acid sequence encoding a polypeptide comprising at least 80% sequence identity to any one of SEQ ID NOs:1-3, any one of SEQ ID NOs:10-13, or any one of SEQ ID NOs:17-19; and (b) a heterologous regulatory element operably linked to the nucleic acid sequence.

13. The polynucleotide of claim 12, wherein the polypeptide comprises at least 90% sequence identity to any one of SEQ ID NOs:1-3, SEQ ID NO:10, SEQ ID NO:11, SEQ ID NO:13, or SEQ ID NO:19.

14. An expression construct comprising the polynucleotide of claim 11.

15. An engineered cell comprising the non-natural flavin-dependent oxidase of claim 1.

16. The engineered cell of claim 15, further comprising a cannabinoid biosynthesis pathway enzyme.

17. The engineered cell of claim 16, wherein the cannabinoid biosynthesis pathway enzyme comprises olivetol synthase (OLS), olivetolic acid cyclase (OAC), prenyltransferase, a geranyl pyrophosphate (GPP) biosynthesis pathway enzyme, or a combination thereof.

18. The engineered cell of claim 15, wherein the cell is a bacterial cell, optionally wherein the cell is an Escherichia coli cell.

19. A cell extract or cell culture medium comprising cannabigerolic acid (CBGA), cannabichromenic acid (CBCA), cannabidiolic acid (CBDA), tetrahydrocannabinolic acid (THCA), cannabigerol (CBG), cannabichromene (CBC), cannabidiol (CBD), tetrahydrocannabinol (THC), cannabigerorcinic acid (CBGOA), cannabiorcichromenic acid (CBCOA), cannabidiorcinic acid (CBDOA), tetrahydrocannabiorcinic acid (THCOA), cannabigerivarinic acid (CBGVA), cannabichromevarinic acid (CBCVA), cannabidivarinic acid (CBDVA), tetrahydrocannabivarinic acid (THCVA), cannabigerorcinol (CBGO), cannabichromeorcin (CBCO), cannabidiorcin (CBDO), tetrahydrocannabiorcin (THCO), cannabigerivarinol (CBGV), cannabichromevarin (CBCV), cannabidivarin (CBDV), tetrahydrocannabivarin (THCV), an isomer, analog or derivative thereof, or a combination thereof derived from the engineered cell of claim 15.

20. A method of making a cannabinoid selected from CBCA, CBC, CBCOA, CBCVA, CBCO, CBCV, CBDA, CBD, CBDOA, CBDVA, CBDO, CBDV, THCA, THC, THCOA, THCVA, THCO, THCV, an isomer, analog or derivative thereof, or a combination thereof, comprising: culturing the engineered cell of claim 15.

21-32. (canceled)
